(19) United States
(12) Patent Application Publication
(10) Pub. No.: US 2024/0324264 A1
(43) Pub. Date: Sep. 26, 2024

(54) OLED WITH HYBRID EMISSIVE LAYER

(71) Applicants: The University of Southern California, Los Angeles, CA (US); The Regents of the University of Michigan, Ann Arbor, MI (US)

(72) Inventors: Mark E. Thompson, Los Angeles, CA (US); Abegail Tadle, Los Angeles, CA (US); Muazzam Idris, Los Angeles, CA (US); Karim El Roz, Los Angeles, CA (US); Daniel Sylvinson Muthiah Ravinson, Los Angeles, CA (US); Stephen R. Forrest, Ann Arbor, MI (US); Chan Ho Soh, Ann Arbor, MI (US)

(21) Appl. No.: 18/669,611

(22) Filed: May 21, 2024

Related U.S. Application Data
(63) Continuation of application No. 16/262,408, filed on Jan. 30, 2019, now Pat. No. 12,029,055.
(60) Provisional application No. 62/623,764, filed on Jan. 30, 2018.

(51) Int. Cl. H10K 50/11; C07D 233/58; C07D 401/10; C09K 11/06; H10K 50/125; H10K 85/30; H10K 85/60; H10K 101/00; H10K 101/10; H10K 101/30

(52) U.S. Cl. CPC H10K 50/11; C07D 233/58; C07D 401/10; C09K 11/06; H10K 50/125; H10K 85/322; H10K 85/654; H10K 85/6572; C09K 2211/1018; H10K 2101/10; H10K 2101/27; H10K 2101/30; H10K 2101/90

(57) ABSTRACT

A hybrid emissive layer and OLED incorporating the same are provided. The hybrid emissive layer includes a first material having a triplet state energy level T1_{H} and a singlet state energy level S1_{H}, & second material having a triplet state energy level T1_{F} and a singlet state energy level S1_{F}; and a third material having a triplet state energy level T1_{P} and a single state energy level S1_{P}, where T1_{F}≥T1_{H}; S1_{F}≤S1_{H}; and T1_{P}<T1_{H}.

### CROSS-REFERENCE TO RELATED APPLICATIONS

This application claims the priority benefit of U.S. Provisional Patent Application No. 62/623,764, filed Jan. 30, 2018, the disclosure of which is incorporated by reference in its entirety.

### STATEMENT REGARDING FEDERALLY SPONSORED RESEARCH OR DEVELOPMENT

This invention was made with government support under Grant No. DE-EE0007077 and DE-EE0008244 awarded by the Department of Energy. The government has certain rights in the invention.

### PARTIES TO A JOINT RESEARCH AGREEMENT

The claimed invention was made by, on behalf of, and/or in connection with one or more of the following parties to a joint university corporation research agreement: Regents of the University of Michigan, Princeton University, University of Southern California, and the Universal Display Corporation. The agreement was in effect on and before the date the claimed invention was made, and the claimed invention was made as a result of activities undertaken within the scope of the agreement.

### BACKGROUND

Opto-electronic devices that make use of organic materials are becoming increasingly desirable for a number of reasons. Many of the materials used to make such devices are relatively inexpensive, so organic opto-electronic devices have the potential for cost advantages over inorganic devices. In addition, the inherent properties of organic materials, such as their flexibility, may make them well suited for particular applications such as fabrication on a flexible substrate. Examples of organic opto-electronic devices include organic light emitting diodes/devices (OLEDs), organic phototransistors, organic photovoltaic cells, and organic photodetectors. For OLEDs, the organic materials may have performance advantages over conventional materials. For example, the wavelength at which an organic emissive layer emits light may generally be readily tuned with appropriate dopants.

OLEDs make use of thin organic films that emit light when voltage is applied across the device. OLEDs are becoming an increasingly interesting technology for use in applications such as flat panel displays, illumination, and backlighting. Several OLED materials and configurations are described in U.S. Pat. Nos. 5,844,363, 6,303,238, and 5,707,745, which are incorporated herein by reference in their entirety.

One application for phosphorescent emissive molecules is a full color display. Industry standards for such a display call for pixels adapted to emit particular colors, referred to as “saturated” colors. In particular, these standards call for saturated red, green, and blue pixels. Alternatively the OLED can be designed to emit white light. In conventional liquid crystal displays emission from a white backlight is filtered using absorption filters to produce red, green and blue emission. The same technique can also be used with OLEDs. The white OLED can be either a single EML device or a stack structure. Color may be measured using CIE coordinates, which are well known to the art.

As used herein, the term “organic” includes polymeric materials as well as small molecule organic materials that may be used to fabricate organic opto-electronic devices. “Small molecule” refers to any organic material that is not a polymer, and “small molecules” may actually be quite large. Small molecules may include repeat units in some circumstances. For example, using a long chain alkyl group as a substituent does not remove a molecule from the “small molecule” class. Small molecules may also be incorporated into polymers, for example as a pendent group on a polymer backbone or as a part of the backbone. Small molecules may also serve as the core moiety of a dendrimer, which consists of a series of chemical shells built on the core moiety. The core moiety of a dendrimer may be a fluorescent or phosphorescent small molecule emitter. A dendrimer may be a “small molecule,” and it is believed that all dendrimers currently used in the field of OLEDs are small molecules.

As used herein, “top” means furthest away from the substrate, while “bottom” means closest to the substrate. Where a first layer is described as “disposed over” a second layer, the first layer is disposed further away from substrate. There may be other layers between the first and second layer, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

As used herein, and as would be generally understood by one skilled in the art, a first “Highest Occupied Molecular Orbital” (HOMO) or “Lowest Unoccupied Molecular Orbital” (LUMO) energy level is “greater than” or “higher than” a second HOMO or LUMO energy level if the first energy level is closer to the vacuum energy level. Since ionization potentials (IP) are measured as a negative energy relative to a vacuum level, a higher HOMO energy level corresponds to an IP having a smaller absolute value (an IP that is less negative). Similarly, a higher LUMO energy level corresponds to an electron affinity (EA) having a smaller absolute value (an EA that is less negative). On a conventional energy level diagram, with the vacuum level at the top, the LUMO energy level of a material is higher than the HOMO energy level of the same material. A “higher” HOMO or LUMO energy level appears closer to the top of such a diagram than a “lower” HOMO or LUMO energy level.

As used herein, and as would be generally understood by one skilled in the art, a first work function is “greater than” or “higher than” a second work function if the first work function has a higher absolute value. Because work functions are generally measured as negative numbers relative to vacuum level, this means that a “higher” work function is more negative. On a conventional energy level diagram, with the vacuum level at the top, a “higher” work function is illustrated as further away from the vacuum level in the downward direction. Thus, the definitions of HOMO and LUMO energy levels follow a different convention than work functions.

More details on OLEDs, and the definitions described above, can be found in U.S. Pat. No. 7,279,704, which is incorporated herein by reference in its entirety.

### SUMMARY

According to an embodiment, an organic light emitting diode/device (OLED) is also provided. The OLED can include an anode, a cathode, and an organic layer, disposed between the anode and the cathode. According to an embodiment, the organic light emitting device is incorporated into one or more device selected from a consumer product, an electronic component module, and/or a lighting panel.

In an embodiment, a hybrid emissive layer for use in a device such as an OLED and, more specifically, for use in a white-emitting OLED (WOLED) is provided, which includes a first material having a triplet state energy level T1_{H} and a singlet state energy level S1_{H}; a second material having a triplet state energy level T1_{F} and a singlet state energy level S1_{F}; and a third material having a triplet state energy level T1_{P} and a single state energy level S1_{P}, where T1_{F}≥T1_{H}, S1_{F}≤S1_{H}, and T1_{P}<T1_{H}. The second material may be a fluorescent emissive material such as a fluorescent dopant and/or the third material may be a phosphorescent emissive material such as a phosphorescent dopant.

In one embodiment, the primary emission from the OLED occurs from the first material. In a most preferred embodiment, all the emission from the OLED occurs from the first material. The OLED also may include a second emissive layer, which also may include the first material. In an embodiment, the first and third materials may be or include the same material.

In some embodiments, the singlet and triplet energy levels of the materials may be within specific ranges, or may exhibit specific relationships. For example, T1_{H} may be at least 0.1 eV greater than T1_{P}; T1_{F} may be at least 0.1 eV greater than T1_{H}; S1_{H} may be at least 3.5 eV; T1_{H} may be 2.4 eV to 2.6 eV; and/or T1_{P} is 1.7 to 2.5 eV, or any combination thereof. Furthermore, the energy gap between S1_{F} and T1_{F} may be 0.05 eV to 0.8 eV or 0.1 eV to 0.8 eV. In some embodiments, the fluorescent emissive dopant may have a fluorescence efficiency of at least 60%.

In one embodiment, the second material is selected from compounds selected from the group consisting of Formula A, Formula B, Formula C, and Formula D:

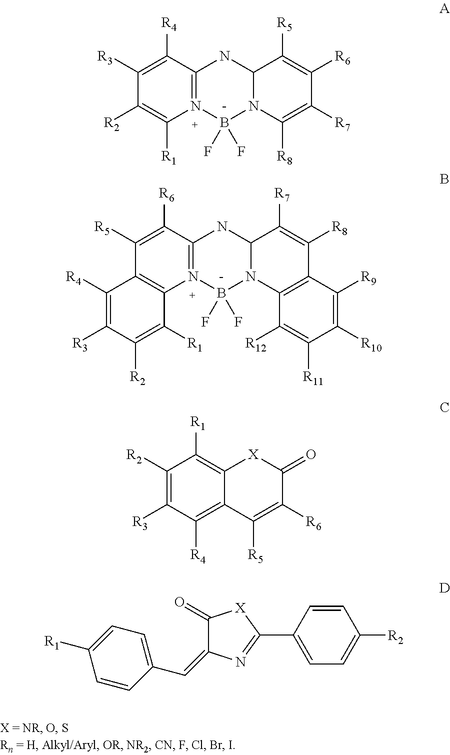

In another embodiment, groups R₁ to R₁₁ for the compounds of Formula A, Formula B, Formula C, and Formula D are independently hydrogen or a substituent selected from the group consisting of deuterium, halogen, alkyl, cycloalkyl, heteroalkyl, heterocycloalkyl, arylalkyl, alkoxy, aryloxy, amino, cyclic amino, silyl, alkenyl, cycloalkenyl, heteroalkenyl, alkynyl, aryl, heteroaryl, acyl, carbonyl, carboxylic acid, ether, ester, nitrile, isonitrile, sulfanyl, sulfinyl, sulfonyl, phosphino, and combinations thereof. Moreover, if X is NR, then R is selected from the group consisting of hydrogen, deuterium, alkyl, cycloalkyl, heteroalkyl, heterocycloalkyl, arylalkyl, silyl, aryl, heteroaryl, and combinations thereof.

In some instances, the group substituents R₁ to R₁₁ are independently selected from the group consisting of deuterium, fluorine, alkyl, cycloalkyl, heteroalkyl, alkoxy, aryloxy, amino, silyl, alkenyl, cycloalkenyl, heteroalkenyl, aryl, heteroaryl, nitrile, isonitrile, sulfanyl, and combinations thereof.

In some instances, the group substituents R₁ to R₁₁ are independently selected from the group consisting of deuterium, fluorine, alkyl, cycloalkyl, alkoxy, aryloxy, amino, silyl, aryl, heteroaryl, and sulfanyl.

In an embodiment, the first material may include a compound selected from the group consisting of:

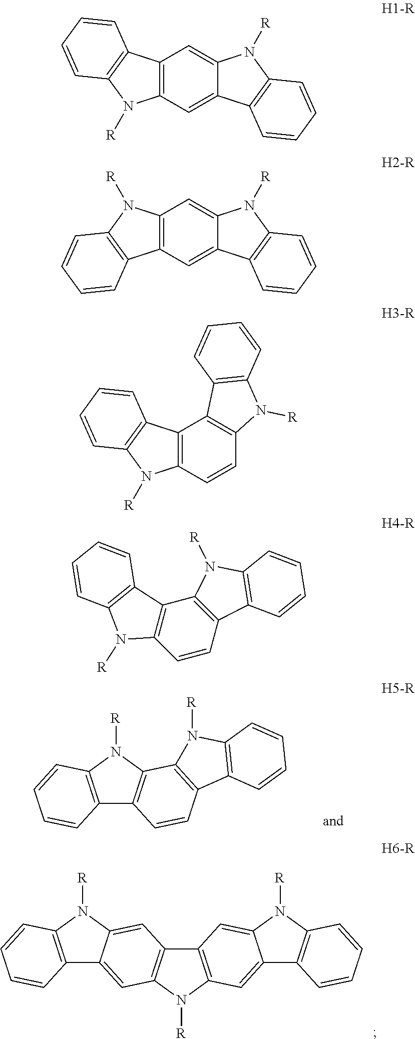

wherein R is selected from the group consisting of hydrogen, deuterium, alkyl, cycloalkyl, heteroalkyl, heterocycloalkyl, arylalkyl, silyl, aryl, heteroaryl, and combinations thereof.

In an embodiment, the first material may include a compound selected from the group consisting of:

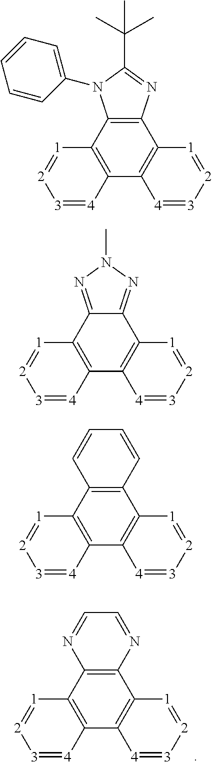

In each of the first material compounds above, ring members 1 to 4 are independently selected from CR^{P} or N, wherein no more than two of ring members 1 to 4 are N; wherein R^{P} is independently hydrogen or a substituent selected from the group consisting of deuterium, halogen, alkyl, cycloalkyl, heteroalkyl, heterocycloalkyl, arylalkyl, alkoxy, aryloxy, amino, cyclic amino, silyl, alkenyl, cycloalkenyl, heteroalkenyl, alkynyl, aryl, heteroaryl, acyl, carbonyl, carboxylic acid, ether, ester, nitrile, isonitrile, sulfanyl, sulfinyl, sulfonyl, phosphino, and combinations thereof. In some instances, the group substituents are independently selected from the group consisting of deuterium, fluorine, alkyl, cycloalkyl, heteroalkyl, alkoxy, aryloxy, amino, silyl, alkenyl, cycloalkenyl, heteroalkenyl, aryl, heteroaryl, nitrile, isonitrile, sulfanyl, and combinations thereof. In an embodiment, the second material may emit light with a peak wavelength of 400 nm to 510 nm.

In an embodiment, a compound is provided of Formula I:

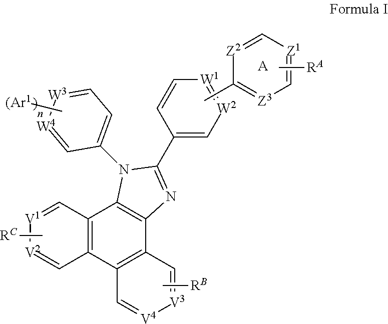

wherein
W¹ and W² are independently selected from C, CH, or N; wherein one of W¹ or W² is C that is substituted with Ring A;
W³ and W⁴ are independently selected from C, CR^{W}, or N, and n is 0 or 1, wherein if n is 1 then one of W³ or W⁴ is C that is substituted with Ar¹;
Z¹, Z², and Z³ are independently selected from CR^{A} or N, and at least one of Z¹, Z², or Z³ is N;
V¹ and V² are independently selected from CR^{C} or N;
V³ and V⁴ are independently selected from CR^{B} or N; and
Ar¹ is selected from an optionally substituted aryl, or an optionally substituted heteroaryl;
R^{W} is independently selected from the group consisting of hydrogen, deuterium, alkyl, cycloalkyl, alkoxy, aryloxy, amino, silyl, heterocyclic, aryl, heteroaryl, nitrile, isonitrile, and combinations thereof;
each R^{A} is independently hydrogen or a substituent selected from the group consisting of deuterium, alkyl, cycloalkyl, alkoxy, aryloxy, amino, silyl, heterocyclic, aryl, heteroaryl, nitrile, isonitrile, and combinations thereof; or optionally, two adjacent R^{A} join to form a fuzed aromatic ring, which is optionally substituted;

R^{B} and R^{C} independently represent from mono substitution to the maximum possible number of substitution, or no substitution; and
each R^{B} and R^{C} is independently hydrogen or a substituent selected from the group consisting of deuterium, alkyl, cycloalkyl, alkoxy, aryloxy, amino, silyl, heterocyclic, aryl, heteroaryl, nitrile, isonitrile, and combinations thereof, or optionally, two adjacent R^{B} or R^{C} join to form a fuzed aromatic ring, which is optionally substituted.

In an embodiment, the compound may be selected from the group consisting of

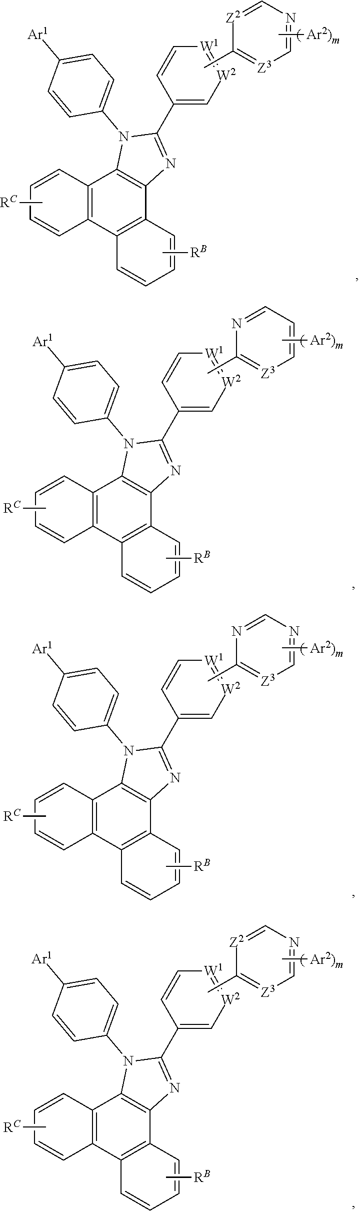

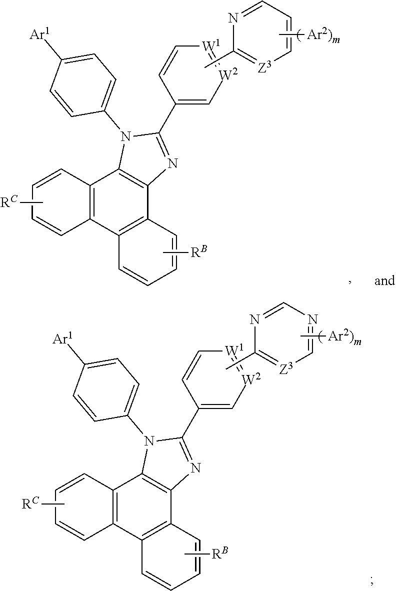

wherein m is 0 or 1, and Ar² is selected aryl or heteroaryl, each of which is optionally substituted.

In an embodiment, Ar¹ and Ar² may be independently selected from the group consisting of

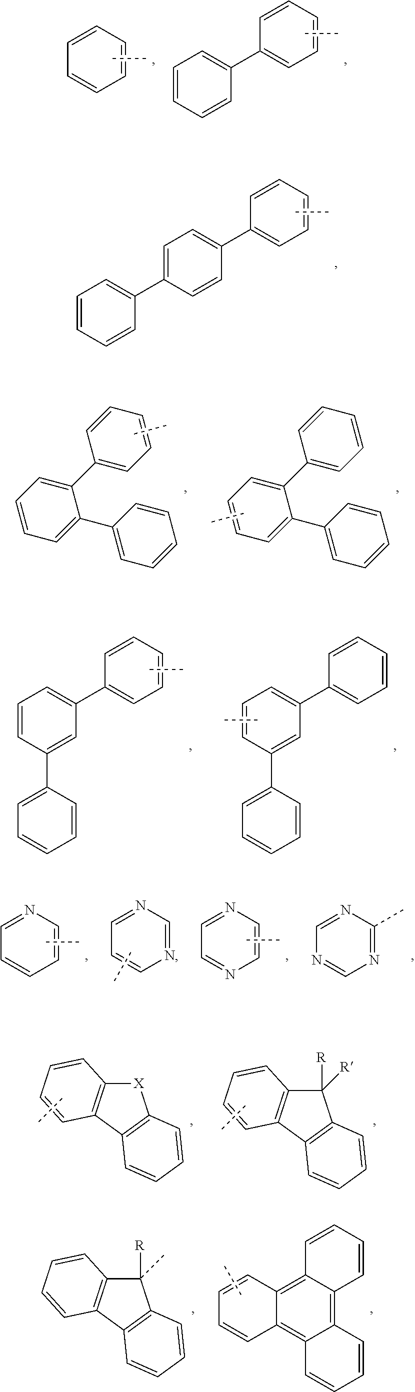

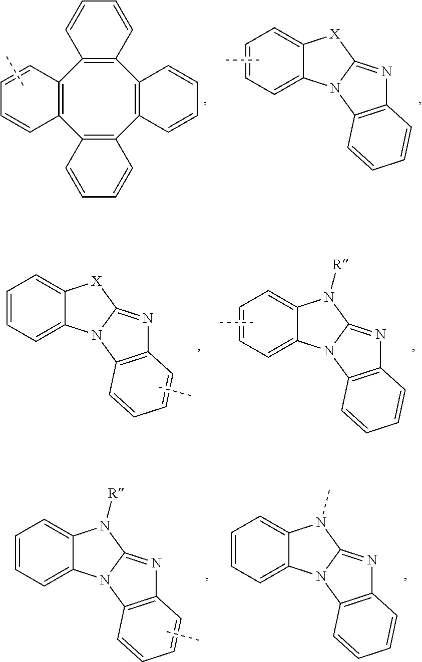

and combinations thereof, or any one aza variant thereof,
wherein X is selected from O, S, or Se;
R, R′, and R″ are independently selected from the group consisting of hydrogen, deuterium, alkyl, cycloalkyl, heteroalkyl, amino, silyl, alkynyl, aryl, heteroaryl, and combinations thereof; and
the dotted line represents attachment to the Ring A.

### BRIEF DESCRIPTION OF THE DRAWINGS

FIG. 1 shows an organic light emitting device.

FIG. 2 shows an inverted organic light emitting device that does not have a separate electron transport layer.

FIG. 3 shows the core structure libraries for fluorescent dopant materials that have been analysed as disclosed herein.

FIG. 4 shows the core structure libraries for host materials according to embodiments disclosed herein.

FIG. 5 shows indolo-carbazole systems with mesityl substitutions on the nitrogen atoms according to embodiments disclosed herein.

FIG. 6 shows structures of H2P/H3 materials according to embodiments disclosed herein.

FIG. 7 shows structures of triarylamine-based materials according to embodiments disclosed herein.

FIG. 8 a procedure to synthesize azaDiPYR derivatives according to embodiments disclosed herein.

FIGS. 9A and 9B show absorption and emission spectra of azaDiPYR and α-azaDiPYR.

FIG. 10 shows structures and energy level structures for the lowest singlet and triplet states for azaDiPYR and α-azaDiPYR according to embodiments disclosed herein.

FIG. 11 shows a synthesis of fluorophores according to embodiments disclosed herein.

FIG. 12 shows normalized absorption and emission spectra for oxazolone derivatives according to embodiments disclosed herein.

FIG. 13 shows a synthesis of 2-PPT according to embodiments disclosed herein.

FIGS. 14A and 14B show emission spectra for 2-PPT (298K), DTIC(298K), 2-PPT (77K), and DTIC (77K), respectively, referring to plot peaks left-to-right across the graphs, measured in 2-MeTHF (FIG. 14A) and in the solid state (FIG. 14B) according to embodiments disclosed herein.

FIG. 15 shows a conventional synthesis for the preparation of 4P-NPD.

FIG. 16 shows the emission spectra of 4P-NPD according to embodiments disclosed herein.

FIG. 17 shows a synthesis of 4P-NPD according to embodiments disclosed herein.

FIG. 18 shows the results of exciton sensor experiments with DCM2 for singlets and PQIr for triplets according to embodiments disclosed herein.

FIGS. 19A, 19B, and 19C show data from exciton sensing experiments according to embodiments disclosed herein.

FIG. 20A shows emission spectra for a device according to embodiments disclosed herein. FIG. 20B shows a photograph of the device. FIG. 20C shows lifetime data for the device.

FIG. 21 shows a distribution of design parameters for a DIPYR library according to embodiments disclosed herein.

FIG. 22 shows structures for compounds meeting various design criteria according to embodiments disclosed herein.

FIG. 23 shows a distribution of design parameters for a α-DIPYR library according to embodiments disclosed herein.

FIG. 24 shows a structure of α-DIPYR candidates according to embodiments disclosed herein.

FIG. 25 shows structures of complexes selected as blue emitters according to embodiments disclosed herein.

FIG. 26 shows absorption and emission spectra of aD and α-aD taken in 2-methyltetrahydrofuran according to embodiments disclosed herein.

FIG. 27 shows normalized spectra of absorption, fluorescence at 298 K, and phosphorescence emission at 77 K in 2-MeTHF according to embodiments disclosed herein.

FIG. 28 shows normalized spectra of absorption, fluorescent emission at 298 K in solution, and fluorescent emission at 298 K in solid for cationic aCarD and α-aCarD according to embodiments disclosed herein.

FIG. 29 shows example structures of the azaDIPYR core modified either by substitution with electron donor or withdrawing group according to embodiments disclosed herein.

FIG. 30 shows structures for α-aD derivatives according to embodiments disclosed herein.

FIG. 31 shows structures of carDIPYR compounds according to embodiments disclosed herein.

FIG. 32 shows synthesis of phenanthro[9,10-d]imidazoles according to embodiments disclosed herein.

FIG. 33 shows structures of Phenanthro[9,10-d]imidazoles (I1-I6) according to embodiments disclosed herein.

FIG. 34 shows the absorption spectra of compounds I1-I6 in 2-MeTHF according to embodiments disclosed herein.

FIG. 35 shows the photoluminescence spectra of I1-I6 according to embodiments disclosed herein.

FIG. 36 shows cyclic voltammetry of phenanthro[9,10-d]imidazoles according to embodiments disclosed herein.

FIG. 37 shows OLED characteristics of phenanthro[9,10-d]imidazoles as neat emitters according to embodiments disclosed herein.

FIG. 38 shows OLED characteristics of α-aD with phenanthro[9,10-d]imidazoles as hosts according to embodiments disclosed herein.

FIG. 39 shows WOLED characteristics of α-aD with I5 as a host material according to embodiments disclosed herein.

FIG. 40 shows WOLED characteristics of I5 as a neat emitter according to embodiments disclosed herein.

FIG. 41 shows WOLED characteristics of I5 as a neat emitter according to embodiments disclosed herein.

FIG. 42 shows WOLED characteristics of I5 as a neat emitter with mixed CBP according to embodiments disclosed herein.

### DETAILED DESCRIPTION

Generally, an OLED comprises at least one organic layer disposed between and electrically connected to an anode and a cathode. When a current is applied, the anode injects holes and the cathode injects electrons into the organic layer(s). The injected holes and electrons each migrate toward the oppositely charged electrode. When an electron and hole localize on the same molecule, an “exciton,” which is a localized electron-hole pair having an excited energy state, is formed. Light is emitted when the exciton relaxes via a photoemissive mechanism. In some cases, the exciton may be localized on an excimer or an exciplex. Non-radiative mechanisms, such as thermal relaxation, may also occur, but are generally considered undesirable.

The initial OLEDs used emissive molecules that emitted light from their singlet states (“fluorescence”) as disclosed, for example, in U.S. Pat. No. 4,769,292, which is incorporated by reference in its entirety. Fluorescent emission generally occurs in a time frame of less than 10 nanoseconds.

More recently, OLEDs having emissive materials that emit light from triplet states (“phosphorescence”) have been demonstrated. Baldo et al., “Highly Efficient Phosphorescent Emission from Organic Electroluminescent Devices,” Nature, vol. 395, 151-154, 1998; (“Baldo-I”) and Baldo et al., “Very high-efficiency green organic light-emitting devices based on electrophosphorescence,” Appl. Phys. Lett., vol. 75, No. 3, 4-6 (1999) (“Baldo-II”), are incorporated by reference in their entireties. Phosphorescence is described in more detail in U.S. Pat. No. 7,279,704 at cols. 5-6, which are incorporated by reference.

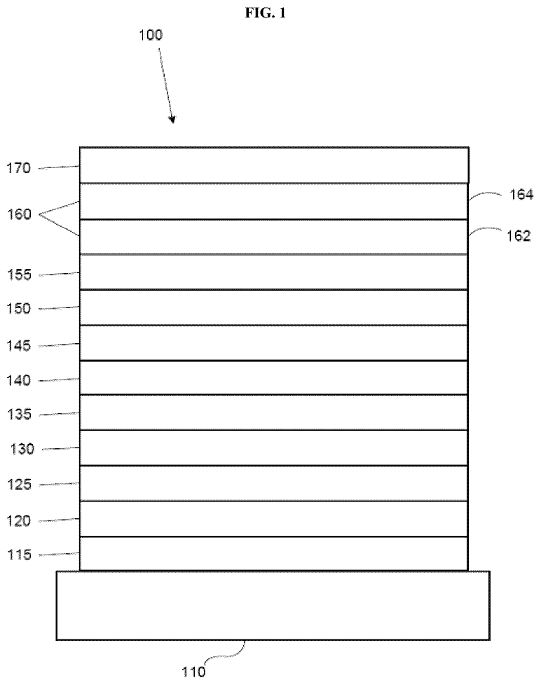

FIG. 1 shows an organic light emitting device 100. The figures are not necessarily drawn to scale. Device 100 may include a substrate 110, an anode 115, a hole injection layer 120, a hole transport layer 125, an electron blocking layer 130, an emissive layer 135, a hole blocking layer 140, an electron transport layer 145, an electron injection layer 150, a protective layer 155, a cathode 160, and a barrier layer 170. Cathode 160 is a compound cathode having a first conductive layer 162 and a second conductive layer 164. Device 100 may be fabricated by depositing the layers described, in order. The properties and functions of these various layers, as well as example materials, are described in more detail in U.S. Pat. No. 7,279,704 at cols. 6-10, which are incorporated by reference.

More examples for each of these layers are available. For example, a flexible and transparent substrate-anode combination is disclosed in U.S. Pat. No. 5,844,363, which is incorporated by reference in its entirety. An example of a p-doped hole transport layer is m-MTDATA doped with F₄-TCNQ at a molar ratio of 50:1, as disclosed in U.S. Patent Application Publication No. 2003/0230980, which is incorporated by reference in its entirety. Examples of emissive and host materials are disclosed in U.S. Pat. No. 6,303,238 to Thompson et al., which is incorporated by reference in its entirety. An example of an n-doped electron transport layer is BPhen doped with Li at a molar ratio of 1:1, as disclosed in U.S. Patent Application Publication No. 2003/0230980, which is incorporated by reference in its entirety. U.S. Pat. Nos. 5,703,436 and 5,707,745, which are incorporated by reference in their entireties, disclose examples of cathodes including compound cathodes having a thin layer of metal such as Mg:Ag with an overlying transparent, electrically-conductive, sputter-deposited ITO layer. The theory and use of blocking layers is described in more detail in U.S. Pat. No. 6,097,147 and U.S. Patent Application Publication No. 2003/0230980, which are incorporated by reference in their entireties. Examples of injection layers are provided in U.S. Patent Application Publication No. 2004/0174116, which is incorporated by reference in its entirety. A description of protective layers may be found in U.S. Patent Application Publication No. 2004/0174116, which is incorporated by reference in its entirety.

FIG. 2 shows an inverted OLED 200. The device includes a substrate 210, a cathode 215, an emissive layer 220, a hole transport layer 225, and an anode 230. Device 200 may be fabricated by depositing the layers described, in order. Because the most common OLED configuration has a cathode disposed over the anode, and device 200 has cathode 215 disposed under anode 230, device 200 may be referred to as an “inverted” OLED. Materials similar to those described with respect to device 100 may be used in the corresponding layers of device 200. FIG. 2 provides one example of how some layers may be omitted from the structure of device 100.

The simple layered structure illustrated in FIGS. 1 and 2 is provided by way of non-limiting example, and it is understood that embodiments of the invention may be used in connection with a wide variety of other structures. The specific materials and structures described are exemplary in nature, and other materials and structures may be used. Functional OLEDs may be achieved by combining the various layers described in different ways, or layers may be omitted entirely, based on design, performance, and cost factors. Other layers not specifically described may also be included. Materials other than those specifically described may be used. Although many of the examples provided herein describe various layers as comprising a single material, it is understood that combinations of materials, such as a mixture of host and dopant, or more generally a mixture, may be used. Also, the layers may have various sublayers. The names given to the various layers herein are not intended to be strictly limiting. For example, in device 200, hole transport layer 225 transports holes and injects holes into emissive layer 220, and may be described as a hole transport layer or a hole injection layer. In one embodiment, an OLED may be described as having an “organic layer” disposed between a cathode and an anode. This organic layer may comprise a single layer, or may further comprise multiple layers of different organic materials as described, for example, with respect to FIGS. 1 and 2.

Structures and materials not specifically described may also be used, such as OLEDs comprised of polymeric materials (PLEDs) such as disclosed in U.S. Pat. No. 5,247,190 to Friend et al., which is incorporated by reference in its entirety. By way of further example, OLEDs having a single organic layer may be used. OLEDs may be stacked, for example as described in U.S. Pat. No. 5,707,745 to Forrest et al, which is incorporated by reference in its entirety. The OLED structure may deviate from the simple layered structure illustrated in FIGS. 1 and 2. For example, the substrate may include an angled reflective surface to improve out-coupling, such as a mesa structure as described in U.S. Pat. No. 6,091,195 to Forrest et al., and/or a pit structure as described in U.S. Pat. No. 5,834,893 to Bulovic et al., which are incorporated by reference in their entireties.

Unless otherwise specified, any of the layers of the various embodiments may be deposited by any suitable method. For the organic layers, preferred methods include thermal evaporation, ink-jet, such as described in U.S. Pat. Nos. 6,013,982 and 6,087,196, which are incorporated by reference in their entireties, organic vapor phase deposition (OVPD), such as described in U.S. Pat. No. 6,337,102 to Forrest et al., which is incorporated by reference in its entirety, and deposition by organic vapor jet printing (OVJP), such as described in U.S. Pat. No. 7,431,968, which is incorporated by reference in its entirety. Other suitable deposition methods include spin coating and other solution based processes. Solution based processes are preferably carried out in nitrogen or an inert atmosphere. For the other layers, preferred methods include thermal evaporation. Preferred patterning methods include deposition through a mask, cold welding such as described in U.S. Pat. Nos. 6,294,398 and 6,468,819, which are incorporated by reference in their entireties, and patterning associated with some of the deposition methods such as ink-jet and OVJD. Other methods may also be used. The materials to be deposited may be modified to make them compatible with a particular deposition method. For example, substituents such as alkyl and aryl groups, branched or unbranched, and preferably containing at least 3 carbons, may be used in small molecules to enhance their ability to undergo solution processing. Substituents having 20 carbons or more may be used, and 3-20 carbons is a preferred range. Materials with asymmetric structures may have better solution processibility than those having symmetric structures, because asymmetric materials may have a lower tendency to recrystallize. Dendrimer substituents may be used to enhance the ability of small molecules to undergo solution processing.

Devices fabricated in accordance with embodiments of the present invention may further optionally comprise a barrier layer. One purpose of the barrier layer is to protect the electrodes and organic layers from damaging exposure to harmful species in the environment including moisture, vapor and/or gases, etc. The barrier layer may be deposited over, under or next to a substrate, an electrode, or over any other parts of a device including an edge. The barrier layer may comprise a single layer, or multiple layers. The barrier layer may be formed by various known chemical vapor deposition techniques and may include compositions having a single phase as well as compositions having multiple phases. Any suitable material or combination of materials may be used for the barrier layer. The barrier layer may incorporate an inorganic or an organic compound or both. The preferred barrier layer comprises a mixture of a polymeric material and a non-polymeric material as described in U.S. Pat. No. 7,968,146, PCT Pat. Application Nos. PCT/US2007/023098 and PCT/US2009/042829, which are herein incorporated by reference in their entireties. To be considered a “mixture”, the aforesaid polymeric and non-polymeric materials comprising the barrier layer should be deposited under the same reaction conditions and/or at the same time. The weight ratio of polymeric to non-polymeric material may be in the range of 95:5 to 5:95. The polymeric material and the non-polymeric material may be created from the same precursor material. In one example, the mixture of a polymeric material and a non-polymeric material consists essentially of polymeric silicon and inorganic silicon.

Devices fabricated in accordance with embodiments of the invention can be incorporated into a wide variety of electronic component modules (or units) that can be incorporated into a variety of electronic products or intermediate components. Examples of such electronic products or intermediate components include display screens, lighting devices such as discrete light source devices or lighting panels, etc, that can be utilized by the end-user product manufacturers. Such electronic component modules can optionally include the driving electronics and/or power source(s). Devices fabricated in accordance with embodiments of the invention can be incorporated into a wide variety of consumer products that have one or more of the electronic component modules (or units) incorporated therein. A consumer product comprising an OLED that includes the compound of the present disclosure in the organic layer in the OLED is disclosed. Such consumer products would include any kind of products that include one or more light source(s) and/or one or more of some type of visual displays. Some examples of such consumer products include flat panel displays, computer monitors, medical monitors, televisions, billboards, lights for interior or exterior illumination and/or signaling, heads-up displays, fully or partially transparent displays, flexible displays, laser printers, telephones, mobile phones, tablets, phablets, personal digital assistants (PDAs), wearable devices, laptop computers, digital cameras, camcorders, viewfinders, micro-displays (displays that are less than 2 inches diagonal), 3-D displays, virtual reality or augmented reality displays, vehicles, video walls comprising multiple displays tiled together, theater or stadium screen, and a sign. Various control mechanisms may be used to control devices fabricated in accordance with the present invention, including passive matrix and active matrix. Many of the devices are intended for use in a temperature range comfortable to humans, such as 18 C to 30 C, and more preferably at room temperature (20-25 C), but could be used outside this temperature range, for example, from −40 C to 80 C.

The materials and structures described herein may have applications in devices other than OLEDs. For example, other optoelectronic devices such as organic solar cells and organic photodetectors may employ the materials and structures. More generally, organic devices, such as organic transistors, may employ the materials and structures.

The terms “halo,” “halogen,” or “halide” as used interchangeably and refer to fluorine, chlorine, bromine, and iodine.

The term “acyl” refers to a substituted carbonyl radical (C(O)—Rₛ).

The term “ester” refers to a substituted oxycarbonyl (—O—C(O)—Rₛ or —C(O)—O—Rₛ) radical.

The term “ether” refers to an —ORₛ radical.

The terms “sulfanyl” or “thio-ether” are used interchangeably and refer to a —SRₛ radical.

The term “sulfinyl” refers to a —S(O)—Rₛ radical.

The term “sulfonyl” refers to a —SO₂—Rₛ radical.

The term “phosphino” refers to a —P(Rₛ)₃ radical, wherein each Rₛ can be same or different.

The term “silyl” refers to a —Si(Rₛ)₃ radical, wherein each Rₛ can be same or different.

In each of the above, Rₛ can be hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, and combination thereof. Preferred Rₛ is selected from the group consisting of alkyl, cycloalkyl, aryl, heteroaryl, and combination thereof.

The term “alkyl” refers to and includes both straight and branched chain alkyl radicals. Preferred alkyl groups are those containing from one to fifteen carbon atoms and includes methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl,and the like. Additionally, the alkyl group may be optionally substituted.

The term “cycloalkyl” refers to and includes monocyclic, polycyclic, and spiro alkyl radicals. Preferred cycloalkyl groups are those containing 3 to 12 ring carbon atoms and includes cyclopropyl, cyclopentyl, cyclohexyl, bicyclo[3.1.1]heptyl, spiro[4.5]decyl, spiro[5.5]undecyl, adamantyl, and the like. Additionally, the cycloalkyl group may be optionally substituted.

The terms “heteroalkyl” or “heterocycloalkyl” refer to an alkyl or a cycloalkyl radical, respectively, having at least one carbon atom replaced by a heteroatom. Optionally the at least one heteroatom is selected from O, S, N, P, B, Si and Se, preferably, O, S or N. Additionally, the heteroalkyl or heterocycloalkyl group is optionally substituted.

The term “alkenyl” refers to and includes both straight and branched chain alkene radicals. Alkenyl groups are essentially alkyl groups that include at least one carbon-carbon double bond in the alkyl chain. Cycloalkenyl groups are essentially cycloalkyl groups that include at least one carbon-carbon double bond in the cycloalkyl ring. The term “heteroalkenyl” as used herein refers to an alkenyl radical having at least one carbon atom replaced by a heteroatom. Optionally the at least one heteroatom is selected from O, S, N, P, B, Si and Se, preferably, O, S or N. Preferred alkenyl, cycloalkenyl, or heteroalkenyl groups are those containing two to fifteen carbon atoms. Additionally, the alkenyl, cycloalkenyl, or heteroalkenyl group is optionally substituted.

The term “alkynyl” refers to and includes both straight and branched chain alkyne radicals. Preferred alkynyl groups are those containing two to fifteen carbon atoms. Additionally, the alkynyl group is optionally substituted.

The terms “aralkyl” or “arylalkyl” are used interchangeably and refer to an alkyl group that is substituted with an aryl group. Additionally, the aralkyl group is optionally substituted.

The term “heterocyclic group” refers to and includes aromatic and non-aromatic cyclic radicals containing at least one heteroatom. Optionally the at least one heteroatom is selected from O, S, N, P, B, Si and Se, preferably, O, S or N. Hetero-aromatic cyclic radicals may be used interchangeably with heteroaryl. Preferred hetero-non-aromatic cyclic groups are those containing 3 to 7 ring atoms which includes at least one hetero atom, and includes cyclic amines such as morpholino, piperidino, pyrrolidino, and the like, and cyclic ethers/thio-ethers, such as tetrahydrofuran, tetrahydropyran, tetrahydrothiophene, and the like. Additionally, the heterocyclic group may be optionally substituted.

The term “aryl” refers to and includes both single-ring aromatic hydrocarbyl groups and polycyclic aromatic ring systems. The polycyclic rings may have two or more rings in which two carbons are common to two adjoining rings (the rings are “fused”) wherein at least one of the rings is an aromatic hydrocarbyl group, e.g., the other rings can be cycloalkyls, cycloalkenyls, aryl, heterocycles, and/or heteroaryls. Preferred aryl groups are those containing six to thirty carbon atoms, preferably six to twenty carbon atoms, more preferably six to twelve carbon atoms. Especially preferred is an aryl group having six carbons, ten carbons or twelve carbons. Suitable aryl groups include phenyl, biphenyl, triphenyl, triphenylene, tetraphenylene, naphthalene, anthracene, phenalene, phenanthrene, fluorene, pyrene, chrysene, perylene, and azulene, preferably phenyl, biphenyl, triphenyl, triphenylene, fluorene, and naphthalene. Additionally, the aryl group may be optionally substituted.

The term “heteroaryl” refers to and includes both single-ring hetero-aromatic groups and polycyclic aromatic ring systems that include at least one heteroatom. The heteroatoms include, but are not limited to O, S, N, P, B, Si and Se. In many instances, O, S or N are the preferred heteroatoms. Hetero-single ring aromatic systems are preferably single rings with 5 or 6 ring atoms, and the ring can have from one to six heteroatoms. The hetero-polycyclic ring systems can have two or more rings in which two atoms are common to two adjoining rings (the rings are “fused”) wherein at least one of the rings is a heteroaryl, e.g., the other rings can be cycloalkyls, cycloalkenyls, aryl, heterocycles, and/or heteroaryls. The hetero-polycyclic aromatic ring systems can have from one to six heteroatoms per ring of the polycyclic aromatic ring system. Preferred heteroaryl groups are those containing three to thirty carbon atoms, preferably three to twenty carbon atoms, more preferably three to twelve carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Of the aryl and heteroaryl groups listed above, the groups of triphenylene, naphthalene, anthracene, dibenzothiophene, dibenzofuran, dibenzoselenophene, carbazole, indolocarbazole, imidazole, pyridine, pyrazine, pyrimidine, triazine, and benzimidazole, and the respective aza-analogs of each thereof are of particular interest.

The terms alkyl, cycloalkyl, heteroalkyl, heterocycloalkyl, alkenyl, cycloalkenyl, heteroalkenyl, alkynyl, aralkyl, heterocyclic group, aryl, and heteroaryl, as used herein, are independently unsubstituted or substituted with one or more general substituents.

In many instances, the general substituents are selected from the group consisting of deuterium, halogen, alkyl, cycloalkyl, heteroalkyl, heterocycloalkyl, arylalkyl, alkoxy, aryloxy, amino, cyclic amino, silyl, alkenyl, cycloalkenyl, heteroalkenyl, alkynyl, aryl, heteroaryl, acyl, carbonyl, carboxylic acid, ether, ester, nitrile, isonitrile, sulfanyl, sulfinyl, sulfonyl, phosphino, and combinations thereof.

In some instances, the preferred general substituents are selected from the group consisting of deuterium, fluorine, alkyl, cycloalkyl, heteroalkyl, alkoxy, aryloxy, amino, silyl, alkenyl, cycloalkenyl, heteroalkenyl, aryl, heteroaryl, nitrile, isonitrile, sulfanyl, and combinations thereof.

In some instances, the preferred general substituents are selected from the group consisting of deuterium, fluorine, alkyl, cycloalkyl, alkoxy, aryloxy, amino, silyl, aryl, heteroaryl, sulfanyl, and combinations thereof.

In yet other instances, the more preferred general substituents are selected from the group consisting of deuterium, fluorine, alkyl, cycloalkyl, aryl, heteroaryl, and combinations thereof.

The terms “substituted” and “substitution” refer to a substituent other than H that is bonded to the relevant position, e.g., a carbon or nitrogen. For example, when R¹ represents mono-substitution, then one R¹ must be other than H (i.e., a substitution). Similarly, when R¹ represents di-substitution, then two of R¹ must be other than H. Similarly, when R¹ represents no substitution, R¹, for example, can be a hydrogen for available valencies of ring atoms, as in carbon atoms for benzene and the nitrogen atom in pyrrole, or simply represents nothing for ring atoms with fully filled valencies, e.g., the nitrogen atom in pyridine. The maximum number of substitutions possible in a ring structure will depend on the total number of available valencies in the ring atoms.

As used herein, “combinations thereof” indicates that one or more members of the applicable list are combined to form a known or chemically stable arrangement that one of ordinary skill in the art can envision from the applicable list. For example, an alkyl and deuterium can be combined to form a partial or fully deuterated alkyl group; a halogen and alkyl can be combined to form a halogenated alkyl substituent; and a halogen, alkyl, and aryl can be combined to form a halogenated arylalkyl. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium. In many instances, a preferred combination of substituent groups will include up to twenty atoms that are not hydrogen or deuterium.

The “aza” designation in the fragments described herein, i.e. aza-dibenzofuran, aza-dibenzothiophene, etc. means that one or more of the C—H groups in the respective fragment can be replaced by a nitrogen atom, for example, and without any limitation, azatriphenylene encompasses both dibenzo[f,h]quinoxaline and dibenzo[f,h]quinoline. One of ordinary skill in the art can readily envision other nitrogen analogs of the aza-derivatives described above, and all such analogs are intended to be encompassed by the terms as set forth herein.

As used herein, “deuterium” refers to an isotope of hydrogen. Deuterated compounds can be readily prepared using methods known in the art. For example, U.S. Pat. No. 8,557,400, Patent Pub. No. WO 2006/095951, and U.S. Pat. Application Pub. No. US 2011/0037057, which are hereby incorporated by reference in their entireties, describe the making of deuterium-substituted organometallic complexes. Further reference is made to Ming Yan, et al., Tetrahedron 2015, 71, 1425-30 and Atzrodt et al., Angew. Chem. Int. Ed. (Reviews) 2007, 46, 7744-65, which are incorporated by reference in their entireties, describe the deuteration of the methylene hydrogens in benzyl amines and efficient pathways to replace aromatic ring hydrogens with deuterium, respectively.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or attached fragment are considered to be equivalent.

The materials and structures described herein may have applications in devices other than OLEDs. For example, other optoelectronic devices such as organic solar cells and organic photodetectors may employ the materials and structures. More generally, organic devices, such as organic transistors, may employ the materials and structures.

In some embodiments, the OLED has one or more characteristics selected from the group consisting of being flexible, being rollable, being foldable, being stretchable, and being curved. In some embodiments, the OLED is transparent or semi-transparent. In some embodiments, the OLED further comprises a layer comprising carbon nanotubes.

In some embodiments, the OLED further comprises a layer comprising a delayed fluorescent emitter. In some embodiments, the OLED comprises a RGB pixel arrangement or white plus color filter pixel arrangement. In some embodiments, the OLED is a mobile device, a hand held device, or a wearable device. In some embodiments, the OLED is a display panel having less than 10 inch diagonal or 50 square inch area. In some embodiments, the OLED is a display panel having at least 10 inch diagonal or 50 square inch area. In some embodiments, the OLED is a lighting panel.

In some embodiments of the emissive region, the emissive region further comprises a host.

In some embodiments, the compound can be an emissive dopant. In some embodiments, the compound can produce emissions via phosphorescence, fluorescence, thermally activated delayed fluorescence, i.e., TADF (also referred to as E-type delayed fluorescence), triplet-triplet annihilation, or combinations of these processes.

The OLED disclosed herein can be incorporated into one or more of a consumer product, an electronic component module, and a lighting panel. The organic layer can be an emissive layer and the compound can be an emissive dopant in some embodiments, while the compound can be a non-emissive dopant in other embodiments.

The organic layer can also include a host. In some embodiments, two or more hosts are preferred. In some embodiments, the hosts used maybe a) bipolar, b) electron transporting, c) hole transporting or d) wide band gap materials that play little role in charge transport. In some embodiments, the host can include a metal complex. The host can be an inorganic compound.

### Combination with Other Materials

The materials described herein as useful for a particular layer in an organic light emitting device may be used in combination with a wide variety of other materials present in the device. For example, emissive dopants disclosed herein may be used in conjunction with a wide variety of hosts, transport layers, blocking layers, injection layers, electrodes and other layers that may be present. The materials described or referred to below are non-limiting examples of materials that may be useful in combination with the compounds disclosed herein, and one of skill in the art can readily consult the literature to identify other materials that may be useful in combination.

Various materials may be used for the various emissive and non-emissive layers and arrangements disclosed herein. Examples of suitable materials are disclosed in U.S. Patent Application Publication No. 2017/0229663, which is incorporated by reference in its entirety.

### Conductivity Dopants

A charge transport layer can be doped with conductivity dopants to substantially alter its density of charge carriers, which will in turn alter its conductivity. The conductivity is increased by generating charge carriers in the matrix material, and depending on the type of dopant, a change in the Fermi level of the semiconductor may also be achieved. Hole-transporting layer can be doped by p-type conductivity dopants and n-type conductivity dopants are used in the electron-transporting layer.

### HIL/HTL

A hole injecting/transporting material to be used in the present invention is not particularly limited, and any compound may be used as long as the compound is typically used as a hole injecting/transporting material.

### EBL

An electron blocking layer (EBL) may be used to reduce the number of electrons and/or excitons that leave the emissive layer. The presence of such a blocking layer in a device may result in substantially higher efficiencies, and or longer lifetime, as compared to a similar device lacking a blocking layer. Also, a blocking layer may be used to confine emission to a desired region of an OLED. In some embodiments, the EBL material has a higher LUMO (closer to the vacuum level) and/or higher triplet energy than the emitter closest to the EBL interface. In some embodiments, the EBL material has a higher LUMO (closer to the vacuum level) and or higher triplet energy than one or more of the hosts closest to the EBL interface. In one aspect, the compound used in EBL contains the same molecule or the same functional groups used as one of the hosts described below.

### Host

The light emitting layer of the organic EL device of the present invention preferably contains at least a metal complex as light emitting material, and may contain a host material using the metal complex as a dopant material. Examples of the host material are not particularly limited, and any metal complexes or organic compounds may be used as long as the triplet energy of the host is larger than that of the dopant. Any host material may be used with any dopant so long as the triplet criteria is satisfied.

### HBL

A hole blocking layer (HBL) may be used to reduce the number of holes and/or excitons that leave the emissive layer. The presence of such a blocking layer in a device may result in substantially higher efficiencies and/or longer lifetime as compared to a similar device lacking a blocking layer. Also, a blocking layer may be used to confine emission to a desired region of an OLED. In some embodiments, the HBL material has a lower HOMO (further from the vacuum level) and or higher triplet energy than the emitter closest to the HBL interface. In some embodiments, the HBL material has a lower HOMO (further from the vacuum level) and or higher triplet energy than one or more of the hosts closest to the HBL interface.

### ETL

An electron transport layer (ETL) may include a material capable of transporting electrons. The electron transport layer may be intrinsic (undoped), or doped. Doping may be used to enhance conductivity. Examples of the ETL material are not particularly limited, and any metal complexes or organic compounds may be used as long as they are typically used to transport electrons.

### Charge Generation Layer (CGL)

In tandem or stacked OLEDs, the CGL plays an essential role in the performance, which is composed of an n-doped layer and a p-doped layer for injection of electrons and holes, respectively. Electrons and holes are supplied from the CGL and electrodes. The consumed electrons and holes in the CGL are refilled by the electrons and holes injected from the cathode and anode, respectively; then, the bipolar currents reach a steady state gradually. Typical CGL materials include n and p conductivity dopants used in the transport layers.

White OLEDs (or WOLEDs) with stacked multiple emissive layers are known and have been reported to produce white light, particularly, for commercial and residential lighting applications. See, for example, U.S. Pub. No. 2006/0006792 to Strip, and U.S. Pat. No. 8,777,291, assigned to Universal Display Corporation. In the UDC '291 Patent, calculations showed that the light emitted for a stacked white OLED is a function of both the wavelength and the source position of the individual light emitting layers. Results indicate that R, G, and B sub-elements, arranged in different orders, have different extraction efficiencies and thus yield different color temperature and color rending indices (CRI) with other parameters staying the same. The emitting layer order of B-G-R (with R adjacent to the ITO anode) is said to lead to an optimal color balance. Although many of such WOLEDs to exhibit high quantum efficiencies (60-70%) and luminous power efficiencies approaching 150 lm/W at brightnesses of 500-1000 cd/m², device performance often falls short of the desired goal, particularly, in terms of device stability or lifetime. One issue arises from a need to improve upon the relatively short operational lifetime of blue phosphorescent emitter dopants, and a consequent loss of color stability for the device.

In 2006, research produced a novel WOLED architecture that minimized many of the shortcomings of an all-phosphorescent OLED resulting in a high efficiency and long lived WOLED. The device employed a fluorescent-emitting dopant to harness all electrically generated high energy singlet excitons for blue emission, and phosphorescent dopants to harvest the remainder of lower-energy triplet excitons for green, red, or vellow emission. Such a structure takes advantage of the fortuitous connection between the proportion of singlets dictated by spin statistics (i.e., one singlet vs. three triplets are produced by electrical excitation) and the roughly 25% contribution of blue to the perceived white light spectrum. The 25:75 spin branching ratio of blue to green+red conforms naturally to the visible spectrum, allowing for WOLEDs that have high color rendering indices (CRIs). Resonant energy transfer from both the host singlet and triplet energy levels minimizes exchange energy losses for both singlet and triplet exciton transfer from the host to each of the dopants, thereby maximizing device power efficiency while maintaining the potential for unity internal quantum efficiency (IQE). This approach has the further advantages of a stable white balance with current, a high efficiency at high brightness due to reduced geminate exciton recombination, and an enhanced lifetime due to the combined use of a stable fluorescent blue, and long lived phosphorescent green and red dopants in a single emissive region. Subsequent research has determined that further improvements and advances in OLED and WOLED technology may be achieved by selecting materials for use in the OLED that have specific energy level relationships.

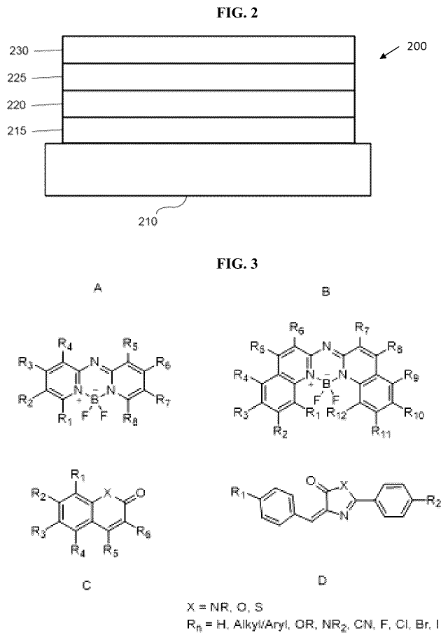

To do so, Density Functional Theory (DFT) calculations may be employed to screen through large libraries of compounds based on critical parameters such as S₁, T₁, HOMO/LUMO energies, and the S₁-T₁ gap (ΔS₁/T₁) to inform and accelerate the materials discovery process. FIG. 3 shows the core structure libraries for fluorescent dopant materials that have been analysed as disclosed herein.

All DFT calculations were performed at the B3LYP/6-31G** level using the Materials Science Suite developed by Schrödinger, LLC. The goal was to identify blue fluorescent dopant materials (“fl-dopants”) that have a relatively small ΔS₁/T₁ (for example, preferably not greater than 0.4 eV) and/or host materials with relatively large ΔS₁/T₁ such that efficient collection of singlet excitons at the fl-dopant and triplet excitons on the phosphorescent dopant materials (“ph-dopants”) is simultaneously possible.

The DFT calculations on the fl-dopant libraries described herein indicate that the coumarin (C) and azlactone (D) based structures have rather large ΔS₁/T₁ due to significant HOMO/LUMO overlap, thereby disqualifying them. The relevant data are provided in Tables 1.1a and 1.1b. The calculations also indicate that the Aza-DIPYR structures (A) may suffer from the existence of a close-lying T₂ state relative to the S₁ state that could potentially quench fluorescence on account of fast symmetry-allowed ISC as reported for a similar dye motif. It has also been found that benzannulation could potentially mitigate this problem by pushing the T₂ state above the S₁ state which has been found to be true for the Aza-alpha-DIPYR structures (B). It is also worth noting that the singlet excited states of the Aza-DIPYR/Alpha-Aza-DIPYR systems have significant multi-reference character that lead to an overestimation of the DFT computed Si energy by approx. 0.44 eV and warrant further investigation using multi-reference methods. A summary of the DFT calculated parameters on all the fl-dopants is shown in Tables 1.1a-1.1d.

TABLE 1,1a
DFT calculated properties for coumarin (C) based dye structures.

| | | S₁ | | T₁ | | HOMO | LUMO | ΔS₁/T₁ |
|---|---|---|---|---|---|---|---|---|
| | | (eV) | S₁ (nm) | (eV) | T₁ (nm) | (eV) | (eV) | (eV) |
| C-1 | 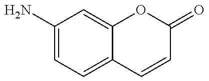 [SMILES: Nc1ccc2ccc(=O)oc2c1] | 3.97 | 312 | 2.59 | 479 | −5.21 | −1.90 | 1.38 |
| C-2 | 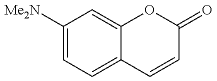 [SMILES: CN(C)c1ccc2ccc(=O)oc2c1] | 3.77 | 329 | 2.52 | 492 | −5.01 | −1.94 | 1.25 |
| C-3 | 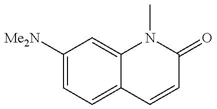 | 3.88 | 320 | 2.67 | 465 | −4.93 | −1.61 | 1.21 |
| C-4 | 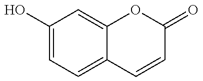 [SMILES: O=c1ccc2ccc(O)cc2o1] | 4.20 | 295 | 2.71 | 457 | −5.82 | −2.09 | 1.49 |
| C-5 | 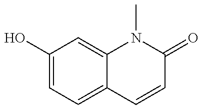 | 4.11 | 302 | 2.84 | 436 | −5.55 | −1.65 | 1.27 |

TABLE 1,1b
DFT calculated properties for azlactone (D) based dye structures.

| | | | | | | HOMO | LUMO | ΔS₁/T₁ |
|---|---|---|---|---|---|---|---|---|
| | | S₁ (eV) | S₁ (nm) | T₁ (eV) | T₁ (nm) | (eV) | (eV) | (eV) |
| JF-1 | 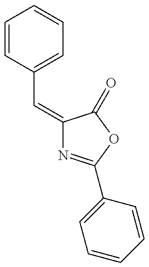 [SMILES: O=C1OC(c2ccccc2)=N/C1=C/c1ccccc1] | 3.34 | 371 | 1.77 | 701 | −5.70 | −2.90 | 1.57 |
| JF-2 | 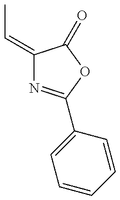 | 4.01 | 309 | 2.13 | 581 | −6.00 | −2.55 | 1.88 |
| JF-3 | 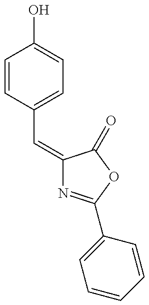 [SMILES: O=C1OC(c2ccccc2)=N/C1=C/c1ccc(O)cc1] | 3.25 | 382 | 1.75 | 710 | −5.40 | −2.75 | 1.50 |
| JF-4 | 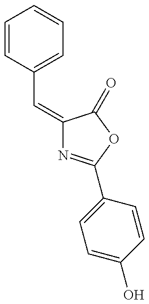 [SMILES: O=C1OC(c2ccc(O)cc2)=N/C1=C/c1ccccc1] | 3.22 | 385 | 1.74 | 711 | −5.46 | −2.83 | 1.48 |
| JF-5 | 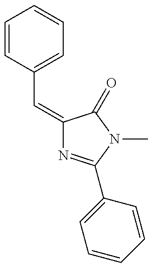 [SMILES: CN1C(=O)/C(=C\c2ccccc2)N=C1c1ccccc1] | 3.20 | 387 | 1.68 | 738 | −5.41 | −2.67 | 1.52 |
| JF-6 | 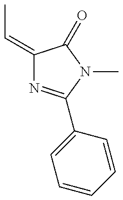 | 3.72 | 334 | 2.21 | 560 | −5.48 | −2.44 | 1.51 |
| JF-7 | 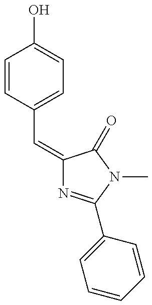 [SMILES: CN1C(=O)/C(=C\c2ccc(O)cc2)N=C1c1ccccc1] | 3.15 | 393 | 1.67 | 742 | −5.17 | −2.53 | 1.48 |
| JF-8 | 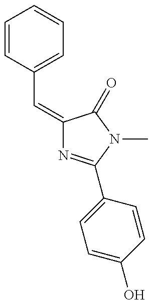 [SMILES: CN1C(=O)/C(=C\c2ccccc2)N=C1c1ccc(O)cc1] | 3.15 | 393 | 1.67 | 744 | −5.23 | −2.60 | 1.48 |

TABLE 1,1c
DFT calculated properties for mono-substituted Aza-alpha-DIPYR structures.

| 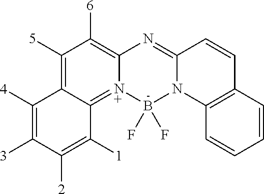 | S₁ (eV) | S₁ (nm) | T₁ (eV) | T₁ (nm) | HOMO (eV) | LUMO (eV) | ΔS₁/T₁ (eV) |
|---|---|---|---|---|---|---|---|
| AAD-OMe-5 | 2.81 | 441 | 2.50 | 496 | −5.51 | −1.85 | 0.31 |
| AAD-F-5 | 2.79 | 444 | 2.47 | 503 | −5.73 | −2.11 | 0.32 |
| AAD-OMe-4 | 2.76 | 449 | 2.43 | 511 | −5.53 | −1.94 | 0.33 |
| AAD-F-4 | 2.75 | 450 | 2.41 | 515 | −5.74 | −2.16 | 0.34 |
| AAD-F-1 | 2.74 | 452 | 2.39 | 520 | −5.67 | −2.10 | 0.36 |
| AAD-F-2 | 2.73 | 454 | 2.39 | 518 | −5.70 | −2.16 | 0.34 |
| AAD-F-6 | 2.71 | 457 | 2.34 | 529 | −5.71 | −2.20 | 0.37 |
| AAD-CN-3 | 2.69 | 460 | 2.37 | 524 | −5.99 | −2.49 | 0.33 |
| AAD-F-3 | 2.68 | 462 | 2.34 | 530 | −5.67 | −2.17 | 0.34 |
| AAD-OMe-2 | 2.68 | 463 | 2.37 | 524 | −5.47 | −1.98 | 0.31 |
| AAD-OMe-1 | 2.68 | 463 | 2.35 | 527 | −5.42 | −1.90 | 0.32 |
| AAD-OMe-6 | 2.66 | 466 | 2.31 | 536 | −5.45 | −1.99 | 0.35 |
| AAD-CN-1 | 2.65 | 468 | 2.34 | 531 | −5.87 | −2.40 | 0.31 |
| AAD-CN-4 | 2.64 | 470 | 2.33 | 533 | −5.97 | −2.51 | 0.31 |
| AAD-CN-2 | 2.63 | 471 | 2.33 | 533 | −5.97 | −2.49 | 0.30 |
| AAD-CN-6 | 2.62 | 473 | 2.35 | 527 | −5.97 | −2.48 | 0.27 |
| AAD-OMe-3 | 2.58 | 480 | 2.26 | 548 | −5.40 | −2.01 | 0.32 |
| AAD-CN-5 | 2.46 | 504 | 2.12 | 584 | −5.97 | −2.70 | 0.34 |

TABLE 1,1d
DFT calculated properties for mono-substituted Aza-alpha-DIPYR structures.

| 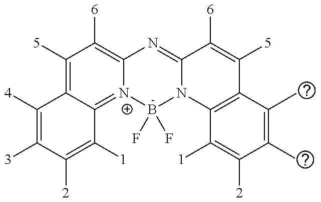 | S₁ (eV) | S₁ (nm) | T₁ (eV) | T₁ (nm) | HOMO (eV) | LUMO (eV) | ΔS₁/T₁ (eV) |
|---|---|---|---|---|---|---|---|
| AAD-(OMe)2-5-5 | 2.92 | 425 | 2.67 | 465 | −5.41 | −1.62 | 0.25 |
| AAD-(F)2-5-5 | 2.85 | 434 | 2.56 | 485 | −5.84 | −2.14 | 0.30 |
| AAD-(OMe)2-4-4 | 2.79 | 444 | 2.47 | 502 | −5.45 | −1.81 | 0.33 |
| AAD-(F)2-4-4 | 2.78 | 446 | 2.43 | 510 | −5.86 | −2.26 | 0.35 |
| AAD-(F)2-1-1 | 2.77 | 448 | 2.39 | 518 | −5.72 | −2.13 | 0.37 |
| AAD-(F)2-2-2 | 2.74 | 453 | 2.40 | 516 | −5.78 | −2.25 | 0.34 |
| AAD-(F)2-6-6 | 2.68 | 462 | 2.30 | 540 | −5.80 | −2.34 | 0.39 |
| AAD-(OMe)2-1-1 | 2.68 | 463 | 2.35 | 529 | −5.25 | −1.71 | 0.33 |
| AAD-(CN)2-3-3 | 2.66 | 466 | 2.36 | 525 | −6.35 | −2.89 | 0.30 |
| AAD-(OMe)2-2-2 | 2.65 | 468 | 2.36 | 525 | −5.36 | −1.90 | 0.29 |
| AAD-(F)2-3-3 | 2.64 | 470 | 2.30 | 539 | −5.73 | −2.28 | 0.34 |
| AAD-(CN)2-6-6 | 2.64 | 470 | 2.37 | 523 | −6.28 | −2.79 | 0.27 |
| AAD-(OMe)2-6-6 | 2.64 | 470 | 2.25 | 550 | −5.31 | −1.90 | 0.38 |
| AAD-(CN)2-4-4 | 2.62 | 473 | 2.31 | 536 | −6.31 | −2.87 | 0.31 |
| AAD-(CN)2-1-1 | 2.62 | 473 | 2.35 | 527 | −6.11 | −2.64 | 0.27 |
| AAD-(CN)2-2-2 | 2.62 | 474 | 2.34 | 531 | −6.30 | −2.83 | 0.28 |
| AAD-(OMe)2-3-3 | 2.49 | 498 | 2.19 | 567 | −5.25 | −1.95 | 0.31 |

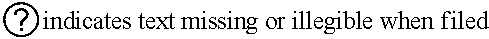

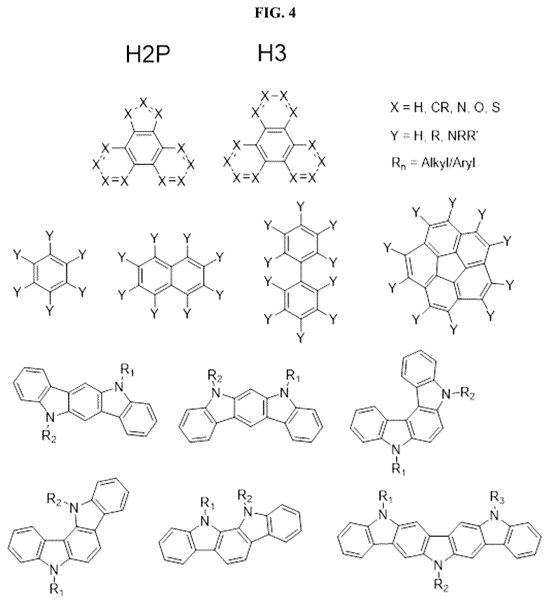

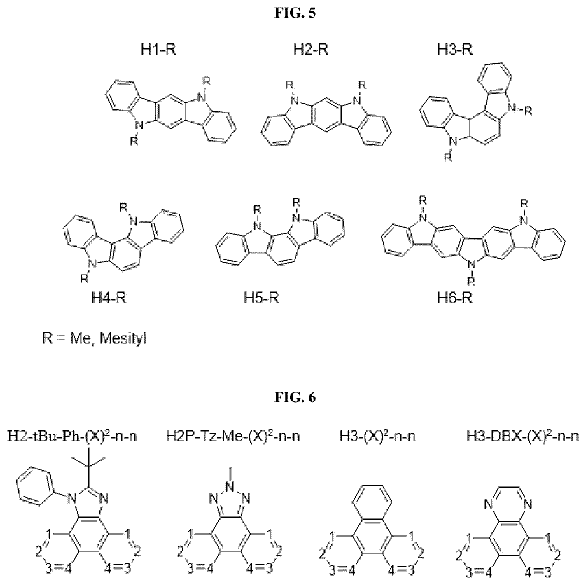

DFT calculations have also been used to identify several prospective host materials across the library shown in FIG. 4, which satisfy the previously-described criteria. DFT calculations were performed on synthetically tractable sub-libraries of indolo-carbazole based materials, H2Ps, H3s, corannulene based structures among others. Among the indolo-carbazole based structures, several promising candidates with high ΔS₁/T₁ and T₁ in the ideal range of 2.4-2.6 e V have been identified and are summarized in Table 2.1a. However, a dramatic red-shifting (˜0.3 eV) of the T₁ state has been observed in the past in the neat solid for several host materials, primarily in the H2P/H3-based systems. It remains to be seen if this is also the case in the indolo-carbazole systems. One way to mitigate this issue may be to add bulky groups onto the core structure, thus hampering effective stacking of the π cores. Therefore, structures with mesityl substitutions on the nitrogen atoms as shown in FIG. 5 have also been explored as described in further detail herein.

TABLE 2.1a
DFT calculated properties for indolo-carbazole based structures.

| | S₁ | S₁ | T₁ | T₁ | HOMO | LUMO | ΔS₁/T₁ |
|---|---|---|---|---|---|---|---|
| | (eV) | (nm) | (eV) | (nm) | (eV) | (eV) | (eV) |
| H1-mes | 3.30 | 376 | 2.77 | 448 | −4.80 | −1.37 | 0.53 |
| H2-mes | 3.72 | 333 | 3.06 | 405 | −4.97 | −1.12 | 0.66 |
| H3-mes | 3.41 | 364 | 2.84 | 437 | −4.82 | −1.33 | 0.57 |
| zH4-mes | 3.77 | 329 | 3.30 | 376 | −4.96 | −0.96 | 0.47 |
| H5-mes | 3.64 | 341 | 3.20 | 388 | −4.93 | −1.21 | 0.44 |
| H6-mes | 2.93 | 423 | 2.49 | 498 | −4.60 | −1.53 | 0.44 |
| H1-Me | 3.30 | 376 | 2.65 | 468 | −4.68 | −1.43 | 0.65 |
| H2-Me | 3.73 | 332 | 2.83 | 438 | −4.85 | −1.16 | 0.90 |
| H3-Me | 3.39 | 366 | 2.69 | 461 | −4.69 | −1.40 | 0.70 |
| H4-Me | 3.71 | 334 | 2.94 | 422 | −4.87 | −1.11 | 0.77 |
| H5-Me | 3.62 | 343 | 2.92 | 425 | −5.15 | −1.25 | 0.70 |

It can be seen in Table 2.1b that several of the H2P/H3 materials, which have structures as shown in FIG. 6, have relatively wide ΔS₁/T₁ gaps. While the T₁ energies seem to be higher (>2.8 eV in most cases) than what is desired (2.4-2.6 eV), several of these materials exhibit a red-shift of about 0.3 eV on average which would make them viable candidates.

TABLE 2.1b
DFT-calculated properties for H2P/H3 class structures
with symmetric Aza substitution patterns.

| | S₁ | S₁ | T₁ | T₁ | HOMO | LUMO | ΔS₁/T₁ |
|---|---|---|---|---|---|---|---|
| | (eV) | (nm) | (eV) | (nm) | (eV) | (eV) | (eV) |
| H2P-Im-tBu-Ph | 3.82 | 325 | 2.85 | 435 | −5.22 | −0.75 | 0.97 |
| H2P-Im-tBu-Ph-(N)2-1-1 | 3.73 | 333 | 2.90 | 427 | −5.68 | −1.26 | 0.82 |
| H2P-Im-tBu-Ph-(N)2-2-2 | 3.55 | 349 | 2.88 | 431 | −5.65 | −1.52 | 0.68 |
| H2P-Im-tBu-Ph-(N)2-3-3 | 4.02 | 309 | 2.91 | 426 | −5.94 | −1.30 | 1.11 |
| H2P-Im-tBu-Ph-(N)2-4-4 | 3.79 | 327 | 2.92 | 425 | −5.59 | −1.14 | 0.87 |
| H2P-Tz-Me | 4.15 | 299 | 2.99 | 414 | −5.90 | −1.09 | 1.15 |
| H2P-Tz-Me-(N)2-1-1 | 4.15 | 299 | 2.99 | 414 | −5.90 | −1.09 | 1.15 |
| H2P-Tz-Me-(N)2-2-2 | 4.15 | 299 | 2.99 | 414 | −5.90 | −1.09 | 1.15 |
| H2P-Tz-meth-(N)2-3-3 | 4.15 | 299 | 2.99 | 414 | −5.90 | −1.09 | 1.15 |
| H2P-Tz-meth-(N)2-4-4 | 4.15 | 299 | 2.99 | 414 | −5.90 | −1.09 | 1.15 |
| H3 | 4.01 | 309 | 2.86 | 434 | −5.86 | −0.95 | 1.15 |
| H3-(N)2-1-1 | 4.00 | 310 | 2.93 | 424 | −6.08 | −1.40 | 1.08 |
| H3-(N)2-2-2 | 3.92 | 316 | 2.91 | 426 | −6.32 | −1.77 | 1.01 |
| H3-(N)2-3-3 | 4.03 | 307 | 2.92 | 425 | −6.39 | −1.65 | 1.11 |
| H3-(N)2-4-4 | 3.97 | 312 | 2.94 | 422 | −6.21 | −1.33 | 1.04 |
| H3-DBX | 3.74 | 331 | 2.88 | 431 | −6.15 | −1.73 | 0.86 |
| H3-DBX-(N)2-1-1 | 3.54 | 350 | 2.90 | 427 | −6.55 | −1.83 | 0.64 |
| H3-DBX-(N)2-2-2 | 3.51 | 354 | 2.97 | 418 | −6.64 | −2.23 | 0.54 |
| H3-DBX-(N)2-3-3 | 3.70 | 335 | 2.94 | 421 | −6.65 | −2.30 | 0.76 |
| H3-DBX-(N)2-4-4 | 3.76 | 330 | 2.98 | 416 | −6.65 | −2.01 | 0.79 |

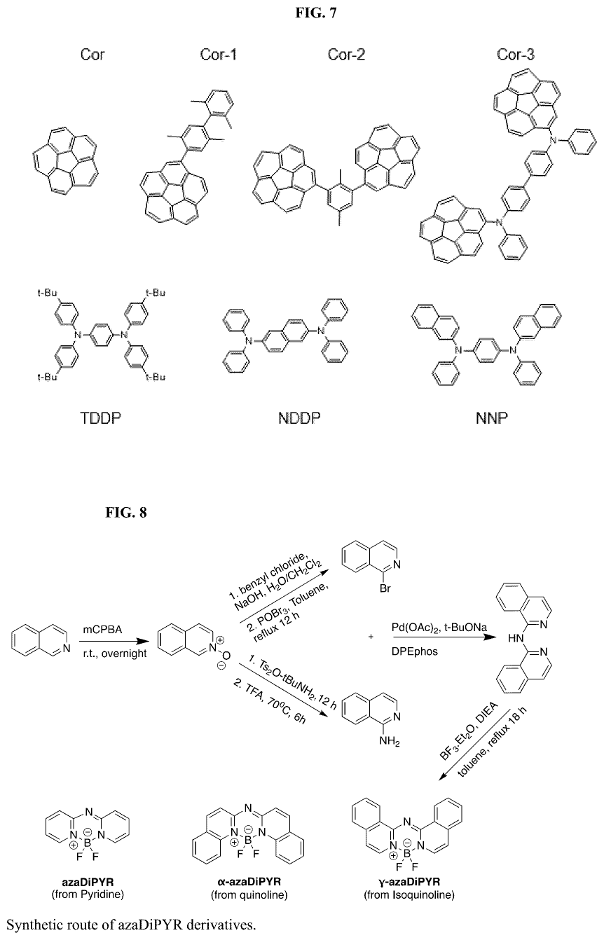

Other classes of material considered herein include corannulene and triaryl amine based structures. The corannulene core may aid efficient charge transport making them very attractive. Triarylamine based materials have been shown to have good hole transport properties and are also routinely used as host and hole transport materials. It can be seen in Table 2.1c that several of these structures, shown in FIG. 7, do have wide ΔS₁/T₁ gaps and T₁ energies in the desired range.

TABLE 2.1c
DFT calculated properties for corannulene based
structures and triarylamine structures.

| | S₁ | S₁ | T₁ | T₁ | HOMO | LUMO | ΔS₁/T₁ |
|---|---|---|---|---|---|---|---|
| | (eV) | (nm) | (eV) | (nm) | (eV) | (eV) | (eV) |
| Cor | 3.54 | 350 | 2.66 | 466 | −5.77 | −1.92 | 0.88 |
| Cor-1 | 3.50 | 354 | 2.62 | 473 | −5.61 | −1.91 | 0.88 |
| Cor-2 | 3.46 | 358 | 2.59 | 479 | −5.67 | −1.91 | 0.87 |
| Cor-3 | 2.75 | 451 | 2.38 | 521 | −4.61 | −1.85 | 0.37 |
| TDDP | 3.49 | 355 | 2.85 | 435 | −4.26 | −0.69 | 0.64 |
| NDDP | 3.10 | 400 | 2.37 | 523 | −4.44 | −1.45 | 0.73 |
| NNP | 3.17 | 391 | 2.49 | 498 | −4.42 | −1.32 | 0.68 |

### Synthesis and Study of fl-Dopants with Small S₁/T₁ Gap

By screening a number of blue fl emitters and estimating their respective S₁, T₁, HOMO, and LUMO energies the best candidates of emitters suitable for hybrid WOLED may be identified. Using this information, promising candidates acting as blue fl-dopants with high quantum efficiencies and small singlet-triplet energy gap (ΔS₁/T₁<0.4 eV) have been successfully synthesized and characterized. Their photophysical and electrochemical properties in both solution and solid matrices have been analyzed as disclosed herein. This combined theoretical/experimental approach allows for rapid identification and targeting of potential candidates that matches the criteria of hybrid blue fl emitters.

In the present work disclosed herein, efforts have focused on synthesizing derivatives of difluoro-boron-azaDiPYR complexes. These complexes were first discovered in the early nineties, but little information is known about their application in OLED devices. Recently, new synthetic procedures of azaDiPYR complexes emerged inspiring a new family of dyes to investigate. FIG. 8 shows the general procedure to successfully synthesize azaDiPYR derivatives in high yields. The target complexes acting as blue fluorescent emitters are azaDiPYR, α-azaDiPYR, and γ-azaDiPYR. These three dyes strongly absorb in the UV-blue part of the spectrum and display blue fluorescence (λₑₘ=400-470 nm) with high quantum yields in solution and doped in thin films (Φ=42%-86%).

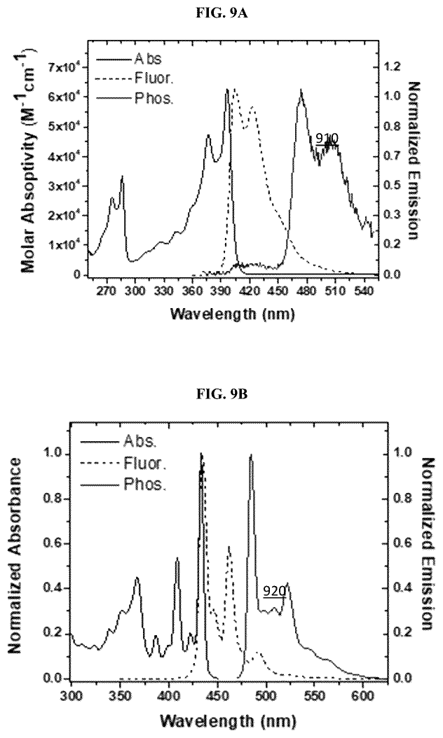

The absorption and emission spectra of azaDiPYR and α-azaDiPYR are shown in FIGS. 9A and 9B. FIG. 9A shows the absorption and normalized emission spectra for azaDiPYR and FIG. 9B shows the same for α-azaDiPYR in MeTHF at room temperature. The gated phosphorescence emission (right-most solid line) was collected at 77 K. Both complexes exhibit strong π to π* absorption bands and intense fluorescence emission in the visible spectrum. The parent azaDiPYR complex has violet-blue fluorescence emission (λ_{em, max}=405 nm) that is the mirror image of the S₀ to S₁ transition (λ_{abs, max}=398 nm). In the UV region, the S₀ to S₂ transition centered at 270 nm also appears as a vibrational absorption feature. For the α-azaDiPYR, both the absorption and emission profile are red shifted compared to the parent azaDiPYR. This information increases the number of potential candidates of host materials with high HOMO and low LUMO energies that can nest the latter blue emitter complex. The gated phosphorescence emission (shown in solid blue lines 910, 920) taken at low temperatures (77 K) represents the lowest triplet energy (T₁).

Table 1.2a compares the singlet and triplet energies for the respective complexes from the calculated methods to the experimental data. The computational data of the singlet-triplet energy gap closely matches the experimental data. The α-azaDiPYR has a small singlet-triplet energy gap of ΔS₁/T₁≤0.3 eV that meets the criteria of developing blue fl-dopants for hybrid WOLED structures and devices, and the parent azaDiPYR has a singlet-triplet energy gap of ΔS₁/T₁≤0.5 eV.

The HOMO and LUMO energies recorded in Table 1.2a were derived from electrochemical data using Ferrocene as an internal standard in acetonitrile solvent.

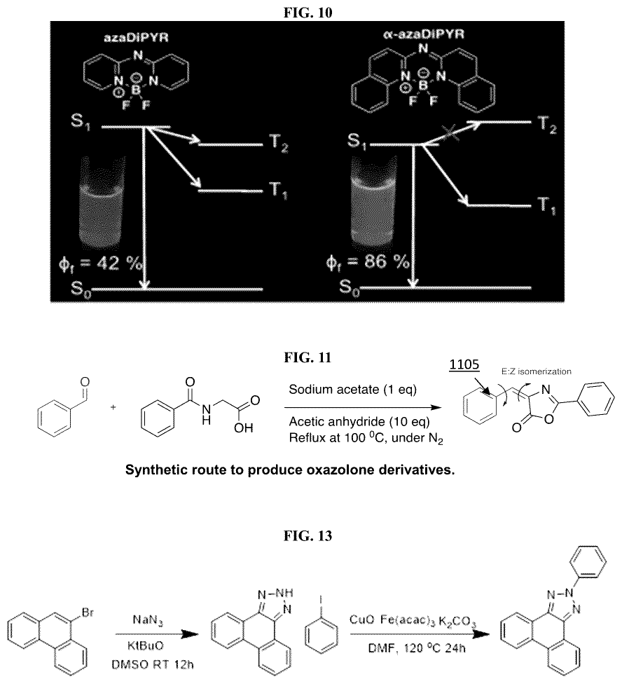

From the DFT calculations, it has also been found that the higher quantum yield of α-azaDiPYR (Φ_{f}=86%) was a result of the relative reordering of the S₁ and T₂ energies upon benzannulation of the parent structure, which is shown in FIG. 10. The reason the parent azaDiPYR has a lower quantum yield (Φ_{f}=42%) is because the energy of T₂ state is just below the S₁ state, enabling fast intersystem crossing that is competitive with fluorescence. All photophysical parameters for azaDiPYR and α-azaDiPYR complexes present in solution and doped in 1 wt % in PMMA films are summarized in Table 1.2b. Notably, the quantum efficiency of α-azaDiPYR remains high in both solution and solid state, maintaining a small singlet-triplet energy gap which is preferable for a blue fluorescent emitter in a hybrid WOLED structure as disclosed herein. FIG. 10 shows a general representation of the lowest singlet and triplet states for azaDiPYR and α-azaDiPYR.

TABLE 1.2a
Theory and experimental data of S₁, T₁, HOMO, and
LUMO energies for azaDiPYR and α-azaDiPYR.

| | azaDiPYR | | α-azaDiPYR | |
|---|---|---|---|---|
| | eV | eV | eV | eV |
| | (calc) | (Exp) | (calc) | (Exp) |
| S₁ | 3.16 | 3.11 | 2.78 | 2.86 |
| T₁ | 2.65 | 2.64 | 2.39 | 2.56 |
| ΔS₁/T₁ | 0.51 | 0.47 | 0.39 | 0.30 |
| HOMO | −5.34 | −5.88 | −5.62 | −6.67 |
| LUMO | −1.84 | −2.06 | −2.07 | −2.42 |

TABLE 1.2b
Photophysical properties of azaDiPYR and α-azaDiPYR
blue fluorescent emitters in solution and solid matrices.

| | | | | 1 wt % |
|---|---|---|---|---|
| | | MeTHF | MeTHF | PMMA |
| | | RT | 77K | at RT |
| azaDiPYR | Φ | 0.42 | — | 0.48 |
| | T (ns) | 2.11 | 2.06 | 2.34 |
| | kᵣ (s) | 2 × 10⁸ | — | 2.05 × 10⁸ |
| | kₙᵣ (s) | 2.74 × 10⁸ | — | 2.22 × 10⁸ |
| α-azaDiPYR | Φ | 0.86 | — | 0.86 |
| | T (ns) | 3.25 | 2.96 | 3.37 |
| | kᵣ (s) | 2.65 × 10⁸ | — | 2.55 × 10⁸ |
| | kₙᵣ (s) | 4.31 × 10⁷ | — | 4.15 × 10⁷ |

To further explore other promising candidates of blue fl emitters, the search may be expanded to study compounds with oxazolone core derivatives. The predications from the modeling study disclosed above suggest that the ΔS₁/T₁ gap will be too large for this family of materials. However, those modeling studies allowed the chromophore to relax in its singlet and triplet excited states. The structure change in the triplet excited state was large relative to the ground state, which will not be possible in the solid state. Accordingly, a few of these dyes were prepared to test them experimentally. These compounds are related to the Green Fluorescent Protein (GFP), which are interesting bioluminescent organisms that originate from the jellyfish Aequorea victoria. Extracting and stabilizing the oxazolone center from the GFP would give an efficient blue fl emitter with attractive photophysical and electrochemical properties. The synthesis of these fluorophores is summarized in FIG. 11. In principle, it is a condensation reaction that can give the desired product in high yields in the presence of an aldehyde and glycine derivatives.

The oxazolone fluorophore is held in a planar conformation in the interior of the GFP, maintaining favorable photophysics and high quantum efficiency of 60%-80%. In solution, these compounds undergo geometrical distortions in the excited states that deviate from the original planar conformation, leading to low fluorescence quantum yield. The PL quantum yields for these dyes were found to be <10% in all cases. Loss of fluorescence energy is mainly due to the free rotation of the aryl-alkene bond resulting in fast non-radiative decay (shown by the arrow 1105 in FIG. 11). The strategy disclosed herein is to introduce bulky groups around the phenyl ring to increase the steric hindrance and slow down or prevent the free rotation of the aryl group. We will also place the fluorophore in thin film matrix such as PMMA solid to stop the free rotation, resulting in a higher fluorescence quantum yield.

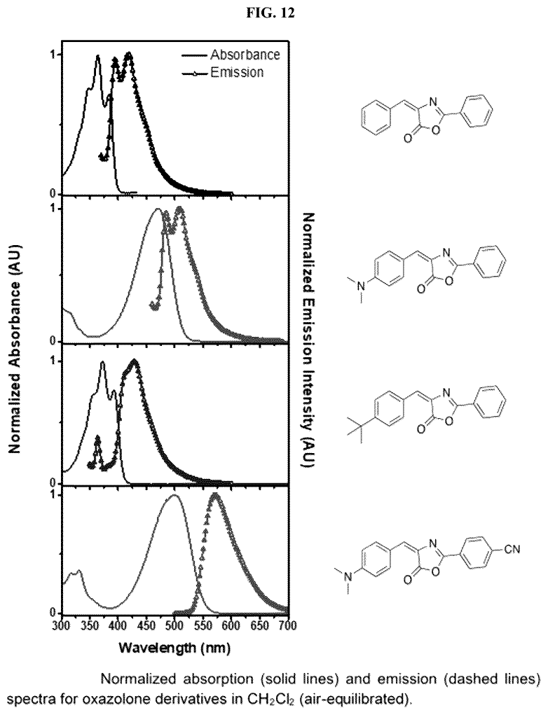

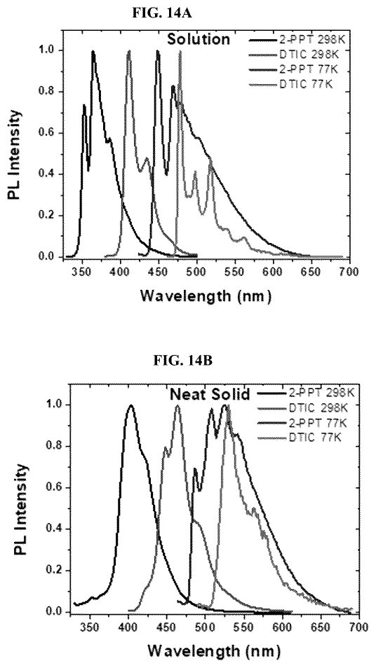

From theoretical modeling, the HOMO is mainly delocalized on the aryl-alkene and the LUMO is on the oxazolone core. In order to lower the singlet-triplet energy gap, we can add functional groups (donating/withdrawing) to minimize the overlap between the HOMO and LUMO, leading to small ΔS₁/T₁. We have synthetically targeted several analogs of oxazolone derivative that would demonstrate this effect, represented in FIG. 12. Compounds with moderately donating groups display an absorption band in the UV region and intense blue fluorescence in the visible spectrum.

We also describe compounds of Formula I.

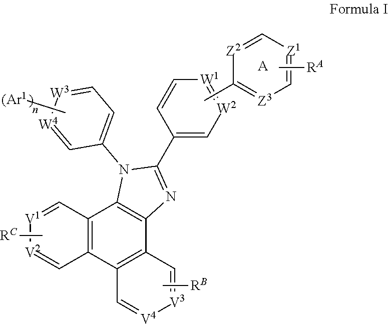

wherein
W¹ and W² are independently selected from C, CH, or N; wherein one of W¹ or W² is C that is substituted with Ring A;
W³ and W⁴ are independently selected from C, CR^{W}, or N, and n is 0 or 1, wherein if n is 1 then one of W³ or W⁴ is C that is substituted with Ar¹;
Z¹, Z², and Z³ are independently selected from CR^{A} or N, and at least one of Z¹, Z², or Z³ is N;
V¹ and V² are independently selected from CR^{C} or N;
V³ and V⁴ are independently selected from CR^{B} or N; and
Ar¹ is selected from an optionally substituted aryl, or an optionally substituted heteroaryl;

R^{W} is independently selected from the group consisting of hydrogen, deuterium, alkyl, cycloalkyl, alkoxy, aryloxy, amino, silyl, heterocyclic, aryl, heteroaryl, nitrile, isonitrile, and combinations thereof;
each R^{A} is independently hydrogen or a substituent selected from the group consisting of deuterium, alkyl, cycloalkyl, alkoxy, aryloxy, amino, silyl, heterocyclic, aryl, heteroaryl, nitrile, isonitrile, and combinations thereof; or optionally, two adjacent R^{A} join to form a fuzed aromatic ring, which is optionally substituted;
R^{B} and R^{C} independently represent from mono substitution to the maximum possible number of substitution, or no substitution; and
each R^{B} and R^{C} is independently hydrogen or a substituent selected from the group consisting of deuterium, alkyl, cycloalkyl, alkoxy, aryloxy, amino, silyl, heterocyclic, aryl, heteroaryl, nitrile, isonitrile, and combinations thereof, or optionally, two adjacent R^{B} or R^{C} join to form a fuzed aromatic ring, which is optionally substituted.

In any one select embodiment of the compounds of Formula I, each R^{B} and R^{C} is independently hydrogen or a substituent selected from the group consisting of deuterium, fluorine, alkyl, cycloalkyl, heteroalkyl, alkoxy, aryloxy, amino, silyl, alkenyl, cycloalkenyl, heteroalkenyl, aryl, heteroaryl, nitrile, isonitrile, and combinations thereof.

Ring A is selected from pyridine (one of Z¹ or Z² is N, preferably, Z¹ is N), or pyrimidine (both Z¹ and Z² are N), or triazine (each of Z¹, Z², and Z³ are N).

In any one of the embodied compounds of Formula I, one of Z¹ or Z² is N, and the other is CR^{A}, which in this case the R^{A} joins with an adjacent R^{A} to from a quinazoline ring.

In one embodiment, W¹ is C that is substituted with Ring A, and W² is CH. In another embodiment, W² is C that is substituted with Ring A, and W¹ is CH.

In any one of the embodied compounds of Formula I, there is particular interest for one R^{A} to be selected from aryl or heteroaryl, each of which is optionally substituted.

In any one of the embodied compounds of Formula I, n is 1, and W³ is C that is substituted with Ar¹. Alternatively, n is 1, and W⁴ is C that is substituted with Ar¹.

In any one of the embodied compounds of Formula I, Ar¹ includes a group selected from the group consisting of

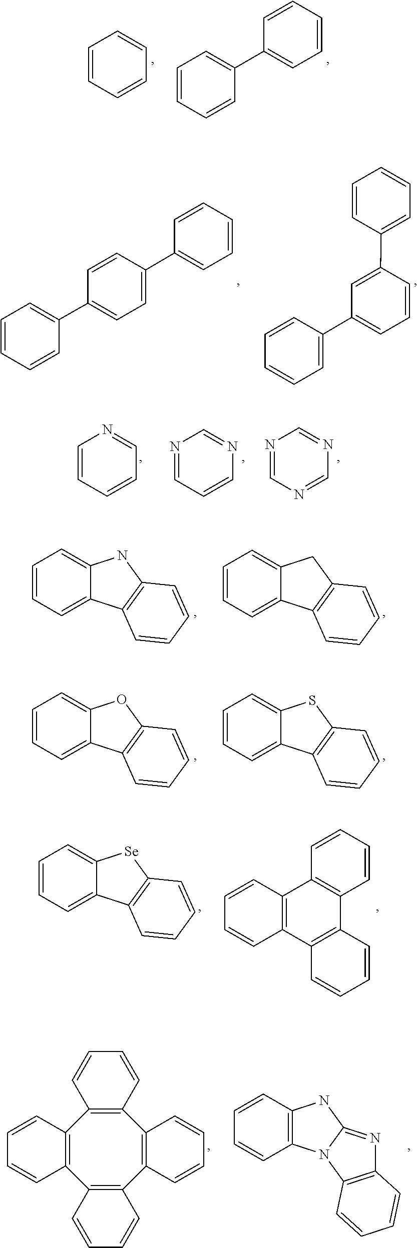

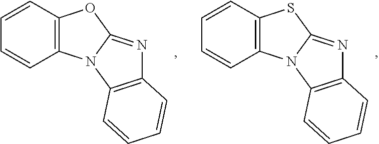 [SMILES: c1ccc2c(c1)nc1oc3ccccc3n12 ; c1ccc2c(c1)nc1sc3ccccc3n12]

and combinations thereof, or any one aza variant thereof.

In any one of the embodied compounds of Formula I, Ar¹ is selected from the group consisting of

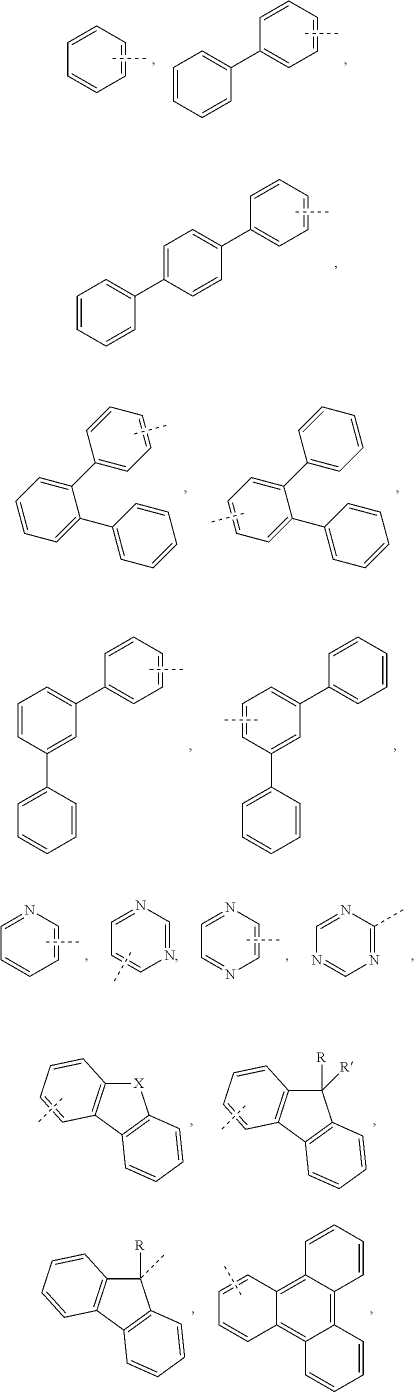

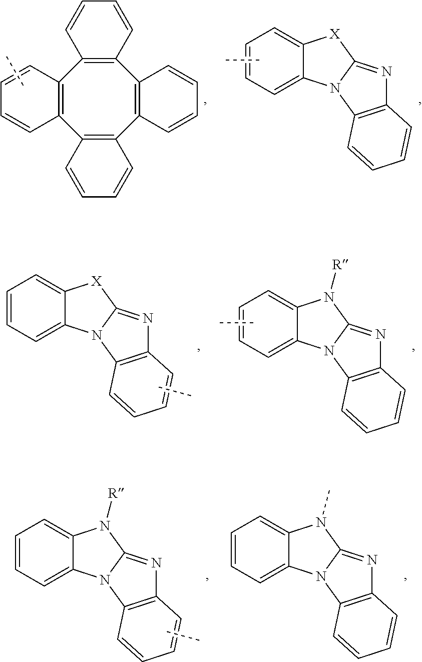

and combinations thercof, or any one aza variant thereof,
wherein X is selected from O, S, or Se;
R, R′, and R″ are independently selected from the group consisting of hydrogen, deuterium, alkyl, cycloalkyl, heteroalkyl, amino, silyl, alkynyl, aryl, heteroaryl, and combinations thereof; and
the dotted line represents attachment to W³ or W⁴.

In any one of the embodied compounds of Formula I, one or two R^{A} is selected from the group consisting of

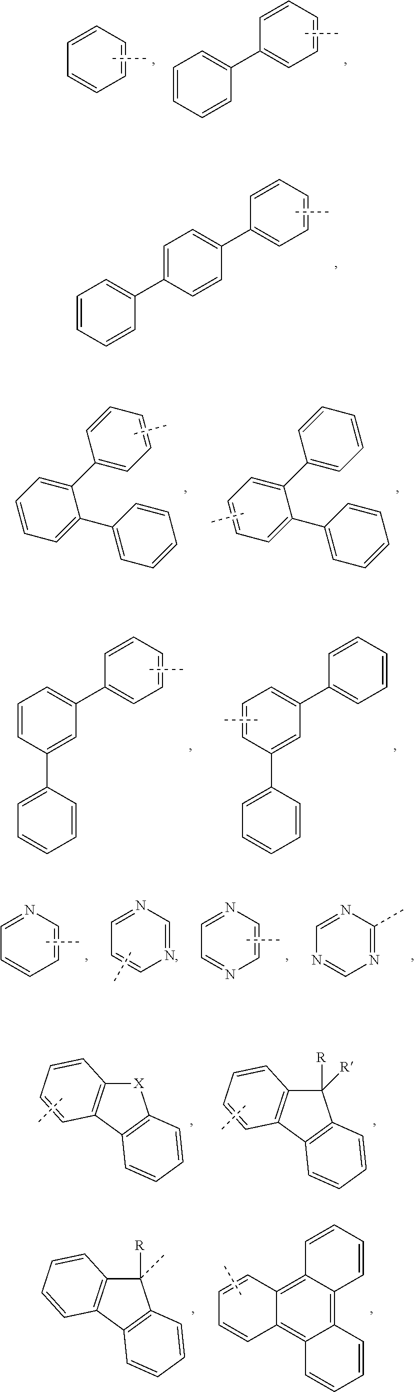

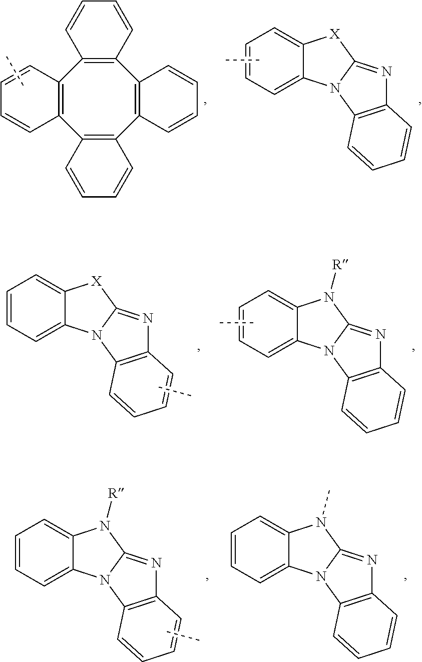

and combinations thereof, or any one aza variant thereof,
wherein X is selected from O, S, or Se;
R, R′, and R″ are independently selected from the group consisting of hydrogen, deuterium, alkyl, cycloalkyl, heteroalkyl, amino, silyl, alkynyl, aryl, heteroaryl, and combinations thereof; and
the dotted line represents attachment to Ring A.

Compounds of Formula I of particular interest will have a structural formula selected from the group consisting of

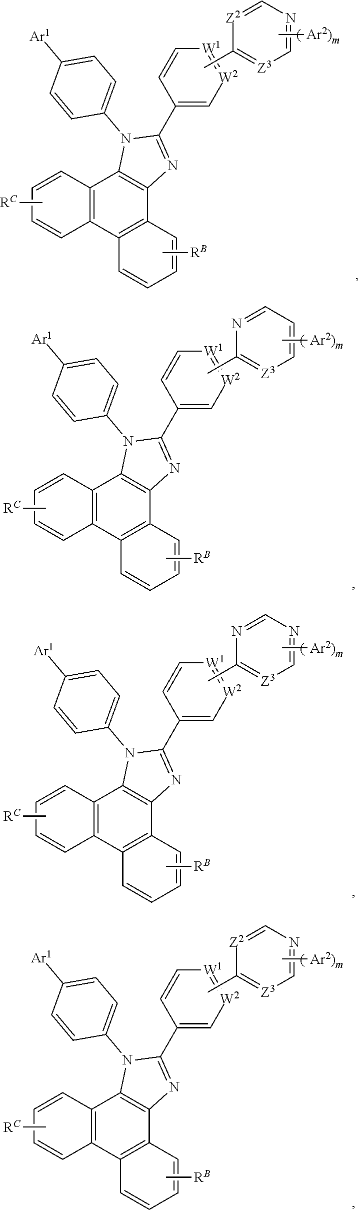

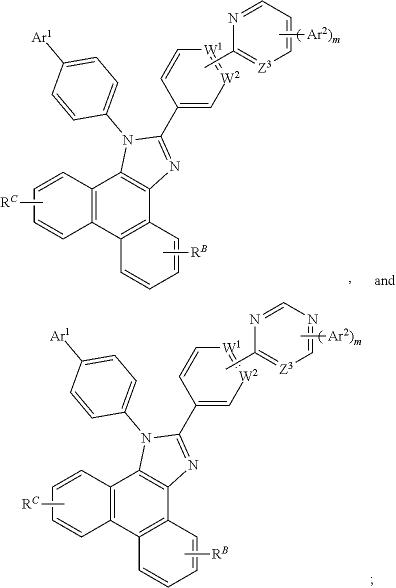

wherein m is 0 or 1, and Ar² is selected aryl or heteroaryl, each of which is optionally substituted.

In any one of the above structural formula of interest, the aromatic ring system Ar² is preferably a group selected from the group consisting of

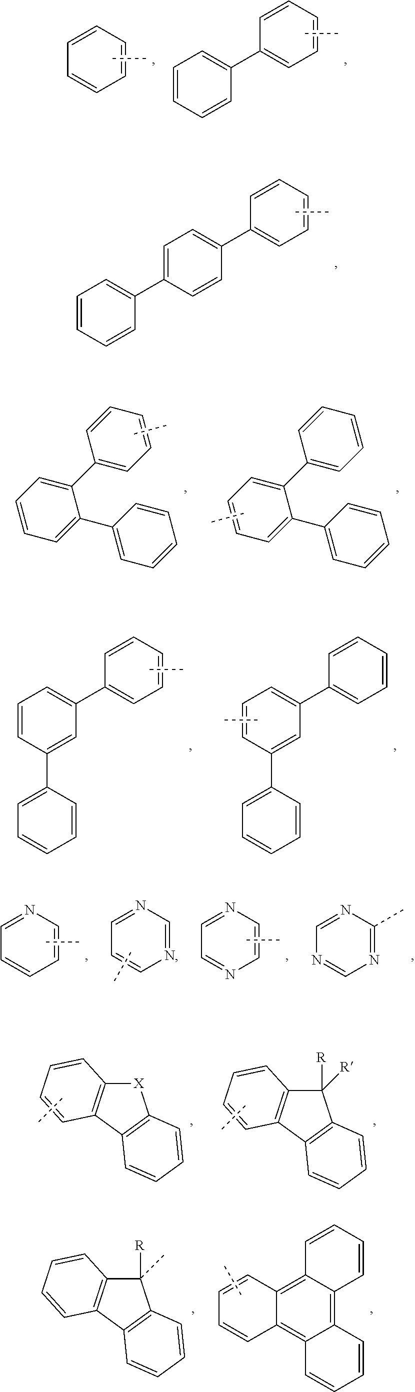

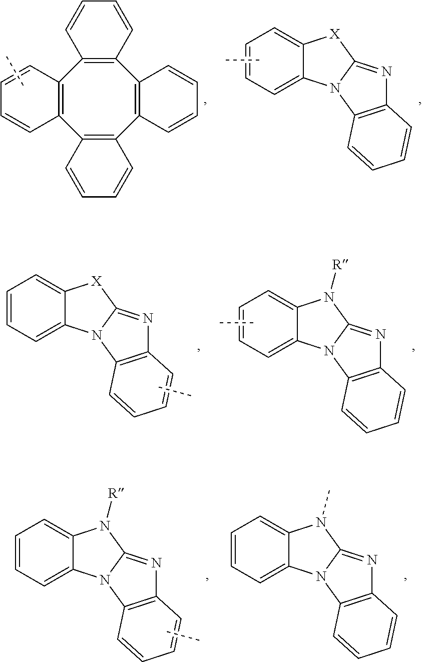

and combinations thereof, or any one aza variant thereof,
wherein X is selected from O, S, or Se;
R, R′, and R″ are independently selected from the group consisting of hydrogen, deuterium, alkyl, cycloalkyl, heteroalkyl, amino, silyl, alkynyl, aryl, heteroaryl, and combinations thereof; and
the dotted line represents attachment to the Ring A.

The invention is also directed to organic light emitting device (OLED) comprising an anode, a cathode, and an organic layer disposed between the anode and the cathode, the organic layer comprising any one described embodied compound of Formula I.

In one embodiment, the the organic layer of the described OLED will be an emissive layer and the compound of Formula I is a host material for a dopant selected from a phosphorescent emissive dopant, a fluorescent emissive dopant, or a TADF emissive dopant.

For example, a phosphorescent emissive dopant of intertest is a transition metal complex having at least one ligand or part of the ligand, if the ligand is more than bidentate, selected from the group consisting of

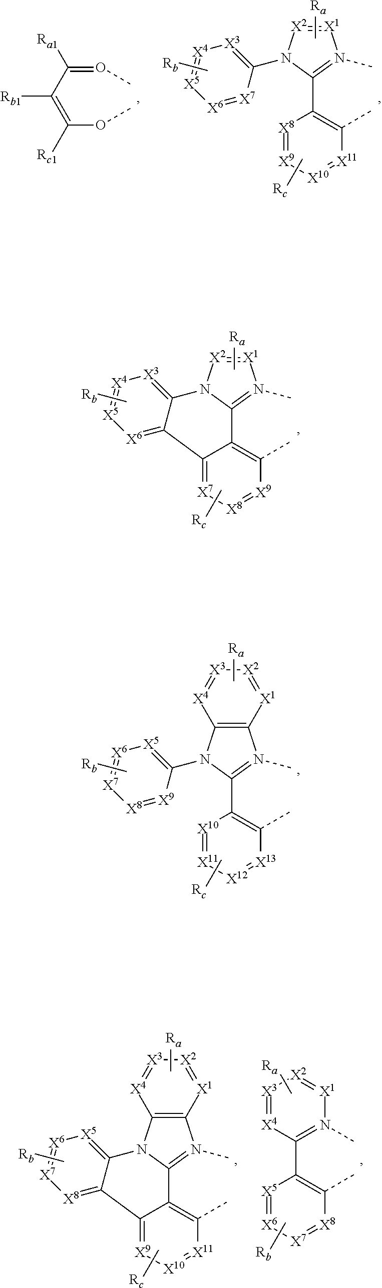

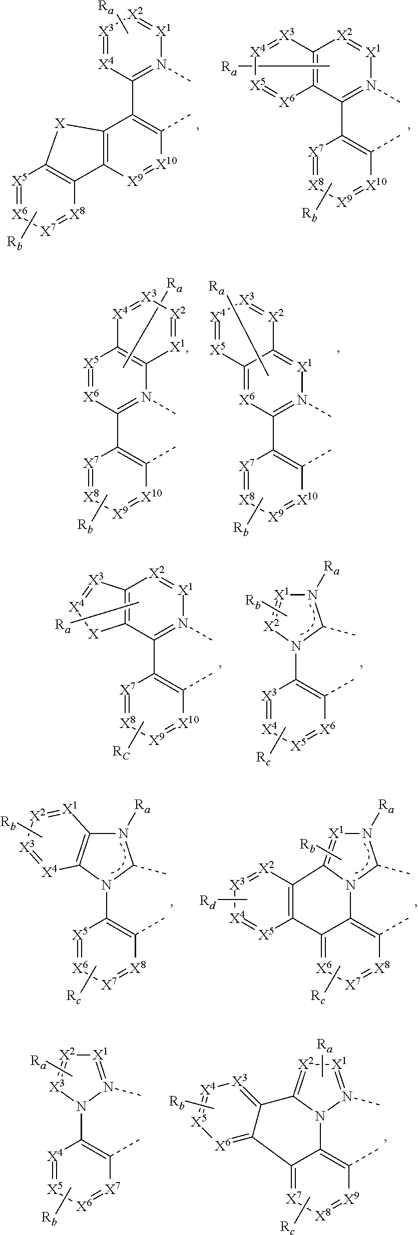

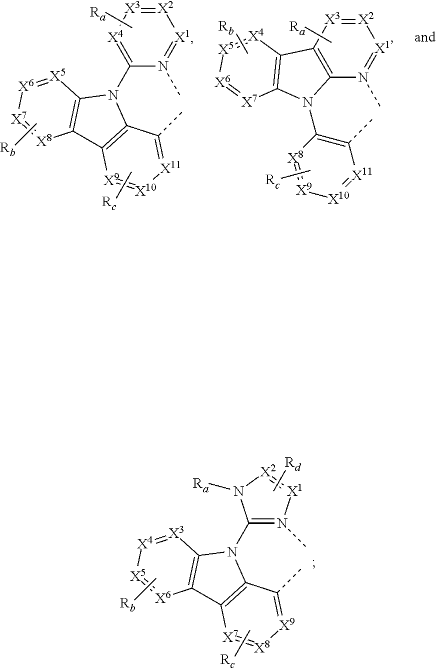

wherein
X¹ to X¹³ are independently selected from C or N;
X is selected from the group consisting of BR′, NR′, PR′, O, S, Se, C═O, S═O, SO₂, CR′R″, SiR′R″, and GeR′R″; wherein R′and R″ are optionally fused or joined to form a ring;
Rₐ, R_{b}, R_{c}, and R_{d} may represent from mono substitution to the possible maximum number of substitution, or no substitution;
R′and R″, and each Rₐ, R_{b}, R_{c}, and R_{d} are independently selected from the group consisting of of hydrogen, deuterium, fluorine, alkyl, cycloalkyl, heteroalkyl, alkoxy, aryloxy, amino, silyl, alkenyl, cycloalkenyl, heteroalkenyl, aryl, heteroaryl, nitrile, isonitrile, and combinations thereof; or optionally, any two adjacent substituents of Rₐ, R_{b}, R_{c}, and R_{d} join to form a ring or join to form a multidentate ligand.

### Host Materials for fl/ph Hybrid WOLEDs

Potential host materials that can nest the energy levels of blue fluorescent dopant and host the singlet and triplet energy of green, yellow and red phosphorescent emitters have also been identified and analyzed, as well as blue fluorescent emitters with strong blue fluorescence in solid state that can act as host material for green, yellow and red phosphors.

To achieve a high luminous efficacy in a WOLED structure, it typically is desirable for the blue fluorescent emitter to have emission centered around 450 nm (˜2.8 eV). Furthermore, the T₁ state of the fluorescent emitter generally should be higher than the T₁ of the host so the triplets generated on the fluorescent emitter can be transferred to the host matrix which will then be transferred to green, yellow and red phosphors. This limits the T₁ state of the blue fluorescent dopant to be around ˜2.6 eV and ˜2.50 eV for the host material. Using DFT calculations as previously disclosed, promising materials that satisfy these criteria have been selected, synthesized and characterized.

As previously indicated, the H2P class of materials show promising properties as host materials. 2-phenyl-2H-phenanthro[9,10-d]triazole (2-PPT) was synthesized and investigated in detail. The preparation of 2-PPT may be carried out in two steps; by benzyne-azide cycloaddition to obtain phenanthro[9,10-d]triazole which is then reacted with iodobenzene via Ullmann coupling to obtain the desired product. The synthesis of 2-PPT is shown in FIG. 13.

The S₁ state of 2-PPT is measured as 3.51 eV which is higher than 2.8 eV of the blue fluorescent dopant satisfying the criterium of high singlet energy. The triplet energy of 2-PPT measured in solution appears to be very high (2.76 eV), but red shifted in solid to 2.59 eV which is low enough for the triplets formed on the blue fluorescent dopant to be transferred to the host and high enough to prevent back energy transfer from the green, yellow and red phosphors to the host, as shown in FIGS. 14A-14B and Table 3a. The LUMO (−1.98 eV) and HOMO (−6.70 eV) levels of 2-PPT are very deep suggesting that it will be electron transporting host. One potential issue with 2-PPT is its low molecular weight which will crystallize on neat film and reduce WOLED lifetime. To mitigate this problem, we dimerize 2-PPT through a meta-bridge phenyl to increase the molecular weight and maintain the photophysical and electrochemical properties.

Another set of potentially-suitable host materials includes indolo carbazoles. From the DFT calculations previously disclosed, H1, H2 and H3 have the right energetics for hosting the emitters in WOLED. One of the indolo-carbazole hosts (H1 with R=phenyl, i.e. DTIC) was synthesized and characterized. The S₁ state (3.02 eV) is high enough to host S₁ state of the emitters, but the triplet energy in neat solid (2.42 eV) is too low to host green emitter (see FIGS. 14A-B and Table 3a). In addition, the HOMO and LUMO levels are too shallow making charge injection barrier very high. Since the triplet spin density is localized on the bicarbazole backbone of DTIC, the triplet energy cannot be increased by addition of other functional groups. Our alternative to DTIC is to synthesize H2 or H3 analogs since their calculated triplet energy is higher than DTIC.

TABLE 3a
Properties of 2-PPT and DTIC

| 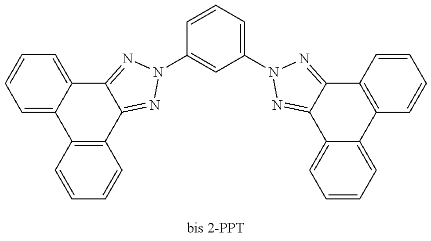 [SMILES: c1cc(-n2nc3c4ccccc4c4ccccc4c3n2)cc(-n2nc3c4ccccc4c4ccccc4c3n2)c1] | S₁ (eV) | T₁ (eV) THF | S₁-T₁ (eV) | T₁ (eV) Neat Solid | ΔT₁ (eV) | HOMO (eV) | LUMO (eV) |
|---|---|---|---|---|---|---|---|
| 2-PPT | 3.51 | 2.76 | 0.75 | 2.59 | 0.17 | −6.71 | −1.98 |
| DTIC | 3.02 | 2.60 | 0.42 | 2.42 | 0.18 | −4.68 | −1.43 |

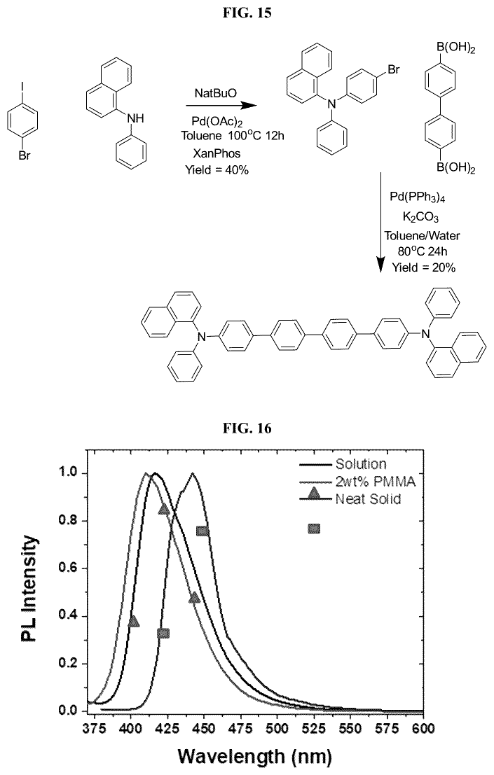

A conventional synthesis for the preparation of 4P-NPD is shown in FIG. 15. The challenge with this synthesis is obtaining the N-(4-bromophenyl)-N-phenylnaphthalen-1-amine precursor in large scale in a very pure form, as impurities carried to the next step decomposes the 4P-NPD upon sublimation.

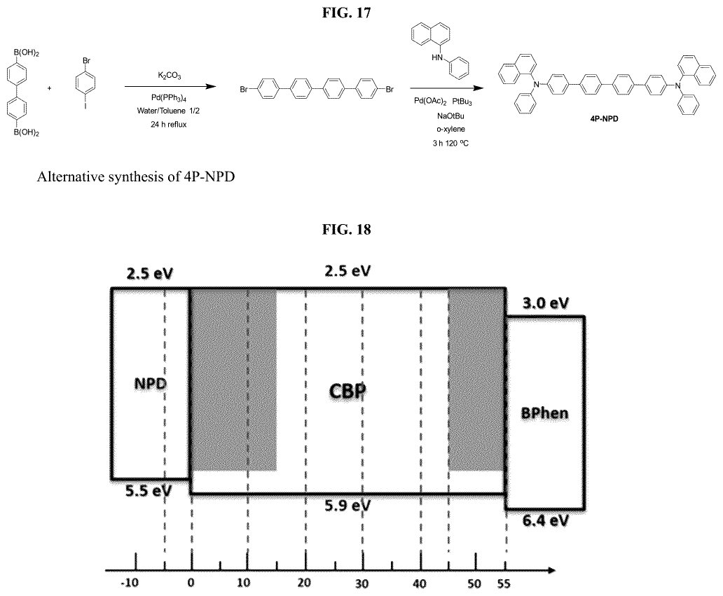

An alternative approach is to make dibroquaterphenyl followed by Ullmann coupling, per the synthesis in FIG. 17. It has been found that dibroquaterphenyl and the byproducts of this reaction were not soluble in common organic solvents, so taking the unpurified precursor yielded 4P-NPD with many byproducts that are hard to separate. Nevertheless, such a process has successfully isolated about 200 mg, sublimed 4P-NPD, and characterize it. The PLQY of 4P-NPD measured previously in the field using comparative method is 92% and this value was confirmed using absolute PLQY measurement as 93%. The emission spectra of 4P-NPD, shown in FIG. 16, is ideal for achieving high luminous efficacy, but the triplet may be too low to host green phosphors in WOLED.

TABLE 3b

| Modelling | | HOMO | LUMO | S₁ (eV, | | T₁ (eV, | □S₁/T₁ |
|---|---|---|---|---|---|---|---|
| DTIC analogs | | (eV) | (eV) | nm) | f | nm) | (eV) |
| DTIC | 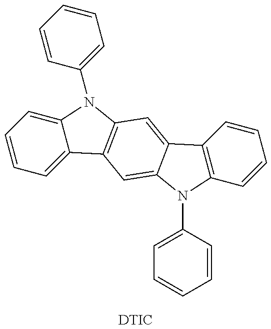 [SMILES: c1ccc(-n2c3ccccc3c3cc4c(cc32)c2ccccc2n4-c2ccccc2)cc1 ; "DTIC"] | −4.85 | −1.03 | 3.28, 378 (3.02, 410)^{a} | 0.04 | 2.66, 466 (2.61, 475)^{a} | 0.62 (0.41)^{a} |
| DTIC-TAZ | 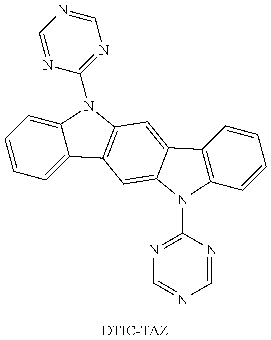 [SMILES: c1ccc2c(c1)c1cc3c(cc1n2-c1ncncn1)c1ccccc1n3-c1ncncn1 ; "DTIC-TAZ"] | −5.69 | −1.62 | 3.54, 350 | 0.14 | 2.73, 455 | 0.81 |
| DTIC- PhTAZ | 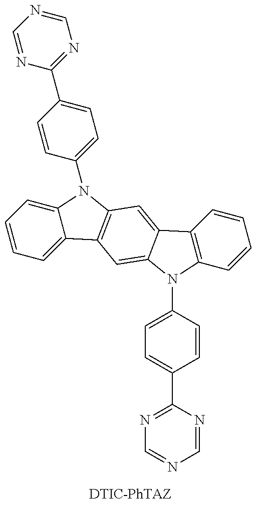 [SMILES: c1ccc2c(c1)c1cc3c(cc1n2-c1ccc(-c2ncncn2)cc1)c1ccccc1n3-c1ccc(-c2ncncn2)cc1] | −5.13 | −2.09 | 2.63, 471 | 0.44 | 2.41, 514 | 0.22 |
| DTIC- Phpyrim | 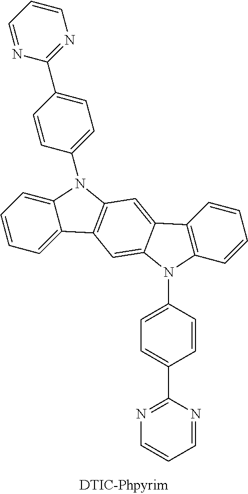 [SMILES: c1cnc(-c2ccc(-n3c4ccccc4c4cc5c(cc43)c3ccccc3n5-c3ccc(-c4ncccn4)cc3)cc2)nc1] | −4.85 | −1.56 | 2.88, 431 | 0.47 | 2.60, 476 | 0.28 |
| DTIC- coumarin | 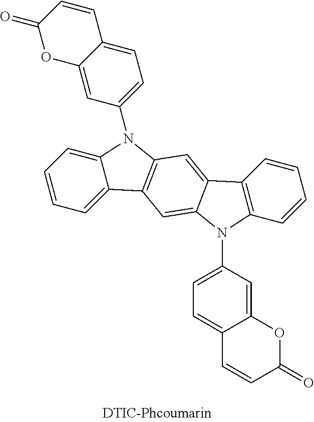 [SMILES: O=c1ccc2ccc(-n3c4ccccc4c4cc5c(cc43)c3ccccc3n5-c3ccc4ccc(=O)oc4c3)cc2o1] | −5.27 | −2.09 | 2.77, 448 | 0.38 | 2.44, 509 | 0.33 |

Table 3b shows materials that have been modelled and that serve as potential neat blue emitter materials. These compounds have higher oscillator strength than DTIC, which suggests they may have higher PLQY. In addition, the frontiers orbitals are stabilized so charge injection barrier will be lowered. It has been found that DTIC-Phpyrim especially has the right S₁ and T₁ state and may provide a suitable neat blue fluorescent emitter candidate for WOLED applications.

### Host materials for fl/ph Hybrid WOLEDs

The fl/ph hybrid WOLEDs developed by Y. Sun, et, al in 2006 as previously described involved doping the blue fluorescent dopant at the EML edges and phosphors in the center of the EML. It has been found that the presence or absence of the phosphor did not affect blue emission, indicating that singlets are formed (and trapped) at the EML edges, and triplets subsequently diffused to the center giving green and red emission.

In the present work disclosed herein, experimental techniques have been applied to directly map out the exciton distribution of the EML in this type of device. The relative exciton densities at each position are obtained by inserting thin δ-doped sensors (˜1 Å) in the EML and quantifying sensor emission. This method minimizes intervention of the sensor. DCM2 and PQIr were used as sensors in this experiment, for mapping singlets and triplets, respectively. FIG. 18 shows the results of exciton sensor experiments with DCM2 for singlets and PQIr for triplets. The dashed lines indicate sensor positions and the shaded regions indicate blue-doped regions.

The singlet profile shown in FIG. 18 clearly shows that excitons are formed at the EML/ETL interface at low current, and are additionally formed at the HTL/EML interface at higher currents. This successfully represents the prediction made by Y. Sun, et al in 2006. Triplets, on the other hand, have higher densities in the EML center. This is evident of triplet diffusion, where they are formed simultaneously with singlets and diffuse to the center.

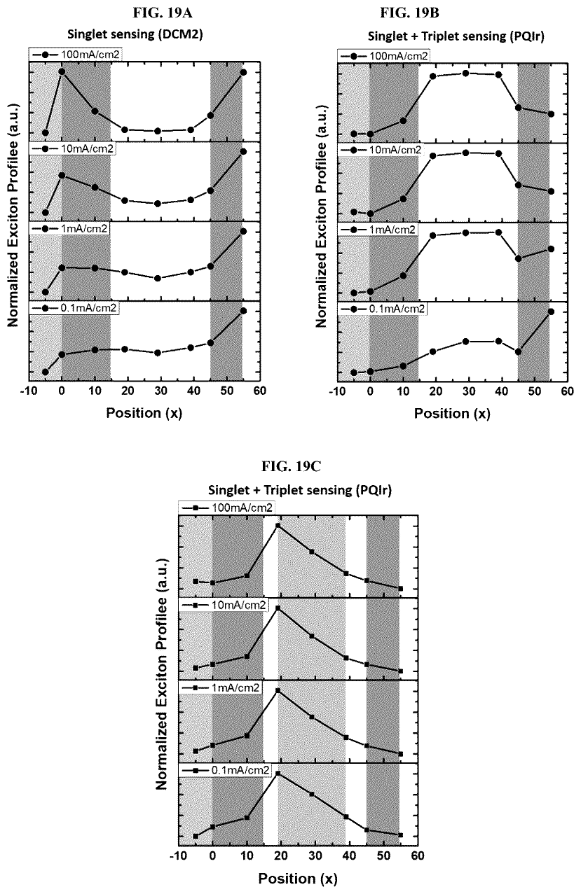

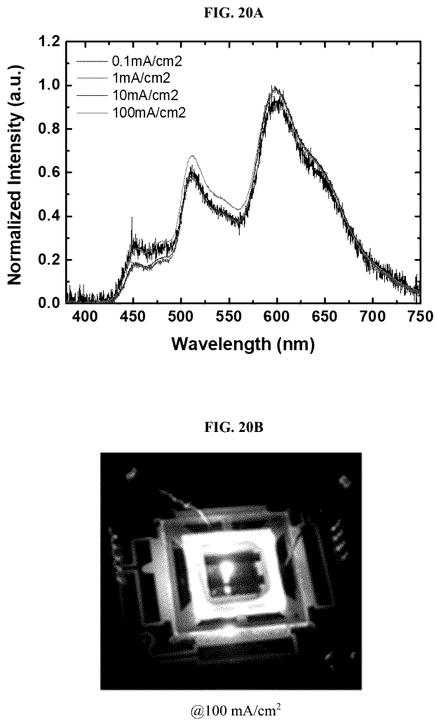

One problem that was found with the prior structure was that phosphors may act as trap site for holes. Y. Sun, et, al previously quantified that 20% of total excitons were formed by direct trapping in phosphors by fitting relative spectra of different dopants to the WOLED spectrum. The analysis disclosed herein reveals that hole trapping effect of phosphors are more significant, indicating that the phosphor doping concentration must be carefully controlled. We have repeated the same sensing experiment with 2 vol % Ir(ppy); placed in the EML center. FIGS. 19A-19C show the exciton populations from the sensing experiments. FIGS. 19A and 19B have no phosphor doping (fl-dopant regions shown in center and right-most shaded regions) and FIG. 19C shows the result of adding a 2% Ir(ppy)₃ dopant to the center of the EML (shown in the third shaded region from the left at x-position of approximately 20-40). The green shaded region indicates Ir(ppy)₃ doping. Unlike the the case with no phosphor, exciton density is highest at the edge of the doped region at all current densities. This shows that holes are trapped by the phosphor and then form excitons when electrons reach the phosphor region edge. This data strongly indicates that a fl/ph hybrid WOLED can be created using this structure.

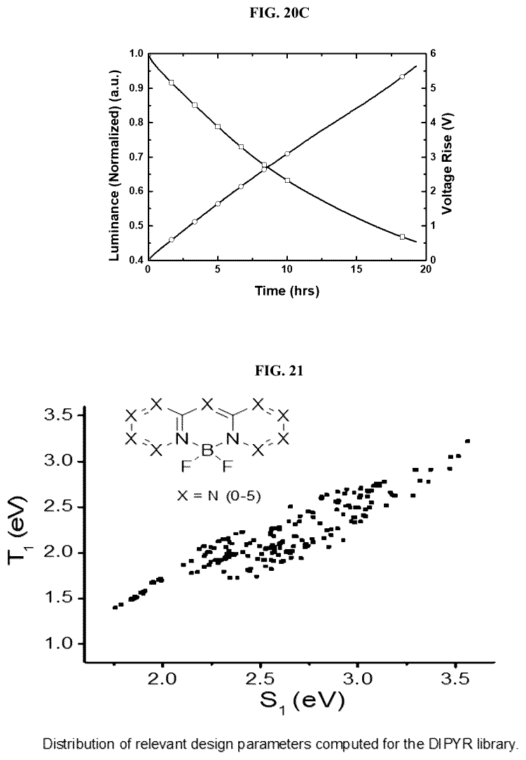

Using this experimental data, the structure of the WOLED was modified for optimized white emission (CIE=[0.44, 0.42], CRI 88.5, CCT 3150K) and the lifetime was measured. The emission spectra at various current densities are shown in FIG. 20A and the lifetime results are shown in FIG. 20C. FIG. 20B shows a photograph of the experimental device. The device showed relatively poor lifetime at initial luminance of 3000 nits at 10 mA/cm2. This may be explained by poor purity of materials used, unstable ETL (BPhen), or blue dopant fragmentation.

Further modelling of blue fluorescent dopants and host materials for hybrid WOLED.

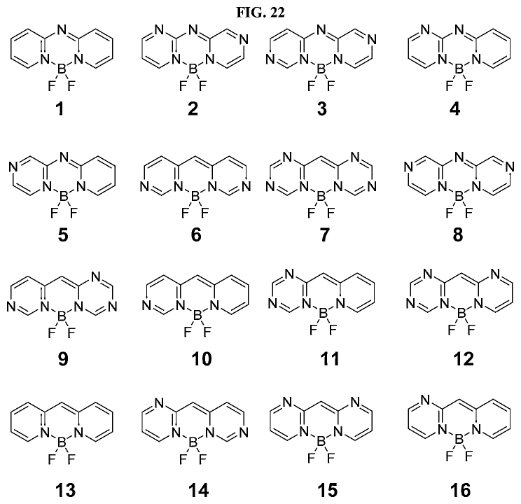

The effect of aza-substitutions on the DIPYR and α-DIPYR core structures have been further analyzed by means of additional DFT calculations. The calculations were performed at the B3LYP/6-31G** level using the Materials Science Suite developed by Schrödinger, LLC. A library containing all possible aza-substitution patterns up to 5 nitrogen substitutions on the positions marked X in FIG. 21 on the DIPYR core structure was developed and DFT calculations (B3LYP/6-31G**) were performed on the entire library to assess their candidacy as fluorophores for the hybrid WOLED architecture. The results of the DFT calculations are summarized in FIG. 21. Based on the DFT results, several candidates that satisfy the primary design criteria previously described (i.e. 3.10>S₁>2.65 eV and T₁>2.18 eV) have been identified. For the screening process, a more relaxed criteria (3.20>S₁>2.50 eV and T₁>2.10 eV) was employed as insurance against errors associated with the calculations. All candidates that satisfied the relaxed criteria are reported in Table 4, with the corresponding structures shown in FIG. 22. Structures that have N atoms adjacent to other N atoms were screened out as such compounds are synthetically infeasible. In all cases, the T₂ state was found to be lower in energy than the S₁ state which has been shown to lead to reduction in the photoluminescence quantum yield (PLQY). The DFT calculated S₁ energies have been subtracted by 0.44 eV to account for the overestimation due to multi-reference character of the singlet excited states.

TABLE 4
Computed parameters of DIPYR candidates that satisfy
the design criteria. All energies are reported in eV.

| | | | | | | Osc. | ΔS₁/ | ΔS₁/ | N- |
|---|---|---|---|---|---|---|---|---|---|
| | HOMO | LUMO | S₁ | T₁ | T₂ | (S₁) | T₁ | T₂ | count |
| 1 | −5.55 | −1.55 | 3.17 | 2.66 | 2.83 | 0.299 | 0.50 | 0.33 | 1 |
| 2 | −6.23 | −2.26 | 3.15 | 2.52 | 2.89 | 0.212 | 0.63 | 0.26 | 3 |
| 3 | −6.36 | −2.41 | 3.14 | 2.52 | 3.01 | 0.257 | 0.62 | 0.13 | 3 |
| 4 | −5.90 | −1.78 | 3.14 | 2.78 | 2.88 | 0.080 | 0.36 | 0.26 | 2 |
| 5 | −5.87 | −2.04 | 3.03 | 2.41 | 2.83 | 0.241 | 0.62 | 0.20 | 2 |
| 6 | −5.80 | −2.07 | 3.01 | 2.34 | 2.84 | 0.307 | 0.67 | 0.18 | 2 |
| 7 | −6.46 | −2.53 | 2.99 | 2.49 | 2.91 | 0.011 | 0.49 | 0.07 | 4 |
| 8 | −6.21 | −2.47 | 2.98 | 2.34 | 2.65 | 0.274 | 0.65 | 0.33 | 3 |
| 9 | −6.12 | −2.29 | 2.92 | 2.42 | 2.85 | 0.045 | 0.51 | 0.07 | 3 |
| 10 | −5.33 | −1.73 | 2.84 | 2.24 | 2.64 | 0.214 | 0.60 | 0.20 | 1 |
| 11 | −5.65 | −1.94 | 2.81 | 2.33 | 2.68 | 0.024 | 0.48 | 0.13 | 2 |
| 12 | −5.96 | −2.18 | 2.77 | 2.39 | 2.67 | 0.032 | 0.38 | 0.10 | 3 |
| 13 | −4.87 | −1.40 | 2.71 | 2.14 | 2.43 | 0.235 | 0.58 | 0.28 | 0 |
| 14 | −5.62 | −1.96 | 2.68 | 2.31 | 2.59 | 0.045 | 0.37 | 0.09 | 2 |
| 15 | −5.46 | −1.82 | 2.60 | 2.28 | 2.44 | 0.016 | 0.32 | 0.16 | 2 |
| 16 | −5.16 | −1.61 | 2.55 | 2.21 | 2.41 | 0.033 | 0.34 | 0.14 | 1 |

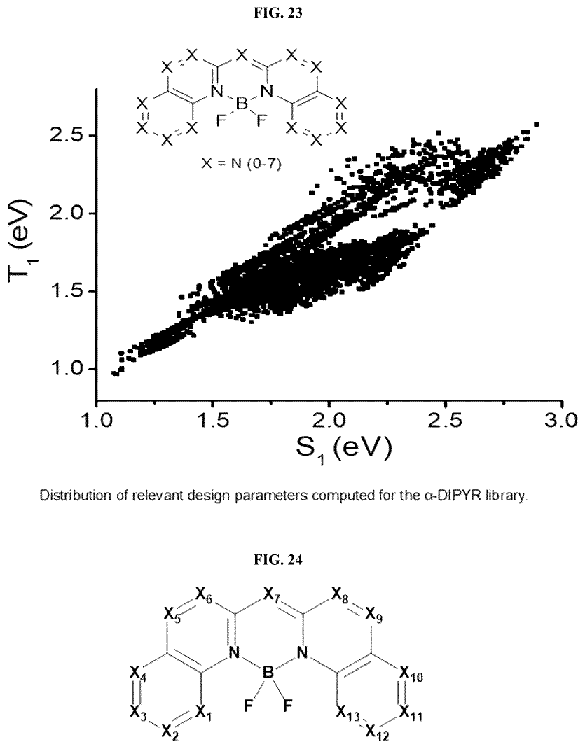

For the α-DIPYR core structure, the library was limited to all possible aza-substitution patterns up to a maximum of 7 nitrogen transmutations in the positions marked ‘X’ in FIG. 23. The most promising candidates that satisfy the relaxed design criteria (3.20>S₁>2.50 eV and T₁>2.10 eV) are listed in Table 5. Again, structures that have N atoms adjacent to other N atoms were screened out. In all cases, the T₂ state is above the S₁ and are hence likely to not suffer from the issue of fluorescence quenching by nonradiative ISC events to which the DIPYR structures are prone. Again, a correction factor of −0.44 eV was applied to the DFT calculated S₁ energies to off-set the errors due to multi-reference nature of the singlet excited states in these systems.

TABLE 5
Computed parameters of α-DIPYR candidates that satisfy the design criteria.
(‘N’ substitution positions follow the numbering scheme above), with
the general structure shown in FIG. 24. All energies are reported in eV.

| | | | | | | | Osc. | ΔS₁/ | ΔS₁/ | N- |
|---|---|---|---|---|---|---|---|---|---|---|
| | N-Positions | HOMO | LUMO | S₁ | T₁ | T₂ | (S₁) | T₁ | T₂ | count |
| 1 | 7-8-11-6-3 | −6.73 | −2.97 | 2.89 | 2.57 | 3.09 | 0.380 | 0.32 | −0.20 | 5 |
| 2 | 7-8-11-6-1-3 | −6.83 | −3.12 | 2.85 | 2.52 | 3.13 | 0.386 | 0.33 | −0.28 | 6 |
| 3 | 7-8-11-13-6-1-3 | −6.92 | −3.24 | 2.84 | 2.52 | 3.10 | 0.431 | 0.31 | −0.26 | 7 |
| 4 | 7-11-6-3 | −6.48 | −2.79 | 2.84 | 2.52 | 2.94 | 0.424 | 0.32 | −0.11 | 4 |
| 5 | 7-8-6-3 | −6.37 | −2.70 | 2.80 | 2.48 | 3.05 | 0.337 | 0.32 | −0.24 | 4 |
| 6 | 7-11-3 | −6.25 | −2.59 | 2.85 | 2.48 | 2.91 | 0.569 | 0.37 | −0.06 | 3 |
| 7 | 7-11-13-6-3 | −6.56 | −2.90 | 2.85 | 2.47 | 3.06 | 0.473 | 0.37 | −0.22 | 5 |
| 8 | 7-8-13-6-3 | −6.48 | −2.84 | 2.77 | 2.47 | 3.05 | 0.350 | 0.30 | −0.27 | 5 |
| 9 | 7-8-11-6-4 | −6.57 | −2.92 | 2.78 | 2.47 | 3.03 | 0.354 | 0.32 | −0.25 | 5 |
| 10 | 7-11-6-1-3 | −6.57 | −2.97 | 2.76 | 2.45 | 2.91 | 0.400 | 0.31 | −0.15 | 5 |
| 11 | 7-6-3 | −6.14 | −2.53 | 2.76 | 2.45 | 2.91 | 0.378 | 0.31 | −0.15 | 3 |
| 12 | 7-8-13-6-1-3 | −6.55 | −2.95 | 2.76 | 2.45 | 3.00 | 0.391 | 0.31 | −0.24 | 6 |
| 13 | 7-11-6 | −6.13 | −2.52 | 2.76 | 2.44 | 2.94 | 0.369 | 0.32 | −0.17 | 3 |
| 14 | 7-13-6-3 | −6.22 | −2.62 | 2.78 | 2.44 | 2.99 | 0.438 | 0.34 | −0.21 | 4 |
| 15 | 7-10-6-3 | −6.32 | −2.72 | 2.76 | 2.44 | 2.99 | 0.414 | 0.32 | −0.23 | 4 |
| 16 | 7-8-11-13-6-4 | −6.66 | −3.05 | 2.77 | 2.43 | 3.01 | 0.380 | 0.34 | −0.24 | 6 |
| 17 | 7-11-1-3 | −6.34 | −2.72 | 2.83 | 2.43 | 2.93 | 0.579 | 0.40 | −0.10 | 4 |
| 18 | 7-12-6-3 | −6.38 | −2.76 | 2.77 | 2.43 | 2.97 | 0.349 | 0.34 | −0.20 | 4 |
| 19 | 7-3 | −5.92 | −2.33 | 2.79 | 2.43 | 2.90 | 0.516 | 0.36 | −0.11 | 2 |
| 20 | 7-8-6 | −6.04 | −2.44 | 2.73 | 2.43 | 2.98 | 0.307 | 0.31 | −0.24 | 3 |
| 21 | 7-11-13-1-3 | −6.41 | −2.83 | 2.81 | 2.42 | 2.98 | 0.604 | 0.39 | −0.18 | 5 |
| 22 | 7-8-6-1-3 | −6.45 | −2.86 | 2.75 | 2.41 | 3.02 | 0.335 | 0.34 | −0.27 | 5 |
| 23 | 7-8-12-6-3 | −6.60 | −2.98 | 2.75 | 2.41 | 3.00 | 0.249 | 0.33 | −0.26 | 5 |
| 24 | 7-10-12-6-3 | −6.59 | −2.98 | 2.78 | 2.41 | 3.01 | 0.383 | 0.36 | −0.23 | 5 |
| 25 | 7-11-6-4 | −6.32 | −2.76 | 2.71 | 2.41 | 2.91 | 0.360 | 0.30 | −0.20 | 4 |
| 26 | 7-11-6-1 | −6.23 | −2.69 | 2.69 | 2.41 | 2.92 | 0.351 | 0.28 | −0.23 | 4 |
| 27 | 7-13-3 | −6.01 | −2.44 | 2.77 | 2.40 | 2.92 | 0.535 | 0.36 | −0.16 | 3 |
| 28 | 7-11-4 | −6.09 | −2.53 | 2.76 | 2.40 | 2.92 | 0.524 | 0.36 | −0.16 | 3 |
| 29 | 7-11-13-6-1 | −6.30 | −2.76 | 2.72 | 2.40 | 2.94 | 0.409 | 0.32 | −0.22 | 5 |
| 30 | 7-8-6-4 | −6.23 | −2.67 | 2.70 | 2.40 | 2.95 | 0.315 | 0.30 | −0.25 | 4 |
| 31 | 7-13-6-1-3 | −6.30 | −2.77 | 2.70 | 2.40 | 2.96 | 0.413 | 0.31 | −0.26 | 5 |
| 32 | 7-9-11-6-3 | −6.76 | −3.14 | 2.80 | 2.40 | 2.88 | 0.448 | 0.41 | −0.08 | 5 |
| 33 | 7-6 | −5.82 | −2.27 | 2.69 | 2.40 | 2.90 | 0.333 | 0.30 | −0.21 | 2 |
| 34 | 7-8-13-6-1 | −6.21 | −2.67 | 2.69 | 2.39 | 2.90 | 0.355 | 0.29 | −0.22 | 5 |
| 35 | 7-8-11-6-2-4 | −6.83 | −3.24 | 2.70 | 2.39 | 2.78 | 0.000 | 0.30 | −0.08 | 6 |
| 36 | 7-8-11-13-6-2-4 | −6.91 | −3.33 | 2.65 | 2.39 | 2.75 | 0.000 | 0.26 | −0.11 | 7 |
| 37 | 7-12-3 | −6.16 | −2.58 | 2.76 | 2.39 | 2.91 | 0.428 | 0.37 | −0.15 | 3 |
| 38 | 7-11-13-6 | 6.21 | −2.62 | 2.77 | 2.39 | 2.98 | 0.423 | 0.39 | −0.21 | 4 |
| 39 | 7 | −5.62 | −2.07 | 2.73 | 2.39 | 2.88 | 0.476 | 0.34 | −0.15 | 1 |
| 40 | 7-8-13-6-4 | −6.31 | −2.77 | 2.69 | 2.39 | 2.91 | 0.343 | 0.30 | −0.22 | 5 |
| 41 | 7-8-10-6-4 | −6.41 | −2.87 | 2.69 | 2.38 | 2.91 | 0.338 | 0.31 | −0.22 | 5 |
| 42 | 7-11-13-6-4 | −6.40 | −2.84 | 2.74 | 2.38 | 2.95 | 0.419 | 0.36 | −0.20 | 5 |
| 43 | 7-8-6-1 | −6.12 | −2.58 | 2.68 | 2.38 | 2.94 | 0.308 | 0.30 | −0.26 | 4 |
| 44 | 7-8-11-13-6-1-4 | −6.76 | −3.24 | 2.66 | 2.38 | 2.71 | 0.000 | 0.28 | −0.05 | 7 |
| 45 | 7-10-6 | −5.99 | −2.46 | 2.69 | 2.38 | 2.93 | 0.368 | 0.31 | −0.23 | 3 |
| 46 | 7-8-12-6-1-3 | −6.68 | −3.10 | 2.73 | 2.38 | 2.94 | 0.300 | 0.36 | −0.21 | 6 |
| 47 | 7-13-1-3 | −6.08 | −2.55 | 2.75 | 2.37 | 2.94 | 0.562 | 0.37 | −0.19 | 4 |
| 48 | 7-12-6 | −6.06 | −2.50 | 2.71 | 2.37 | 2.93 | 0.319 | 0.34 | −0.22 | 3 |
| 49 | 7-10-6-1-3 | −6.40 | −2.89 | 2.69 | 2.37 | 2.99 | 0.385 | 0.32 | −0.30 | 5 |
| 50 | 7-13-6 | −5.89 | −2.35 | 2.71 | 2.37 | 2.92 | 0.395 | 0.34 | −0.21 | 3 |
| 51 | 7-9-11-6-1-3 | −6.83 | −3.29 | 2.74 | 2.36 | 2.82 | 0.432 | 0.37 | −0.09 | 6 |
| 52 | 7-12-6-1-3 | −6.46 | −2.93 | 2.70 | 2.36 | 2.99 | 0.332 | 0.34 | −0.30 | 5 |
| 53 | 7-8-11-6-1-4 | −6.68 | −3.17 | 2.67 | 2.36 | 2.76 | 0.323 | 0.31 | −0.09 | 6 |
| 54 | 7-9-11-13-6-3 | −6.86 | −3.24 | 2.65 | 2.36 | 2.82 | 0.000 | 0.29 | −0.17 | 6 |
| 55 | 7-11-2-4 | −6.35 | −2.81 | 2.74 | 2.36 | 2.91 | 0.432 | 0.38 | −0.17 | 4 |
| 56 | 7-6-1-3 | −6.22 | −2.72 | 2.66 | 2.36 | 2.89 | 0.342 | 0.31 | −0.23 | 4 |
| 57 | 7-9-11-13-6-1-3 | −6.93 | −3.37 | 2.53 | 2.36 | 2.73 | 0.000 | 0.17 | −0.20 | 7 |
| 58 | 7-11-6-2 | −6.35 | −2.82 | 2.66 | 2.36 | 2.89 | 0.240 | 0.31 | −0.23 | 4 |
| 59 | 7-11-13-4 | −6.17 | −2.64 | 2.75 | 2.35 | 2.97 | 0.544 | 0.39 | −0.22 | 4 |
| 60 | 7-1-3 | −6.00 | −2.46 | 2.76 | 2.35 | 2.91 | 0.521 | 0.40 | −0.16 | 3 |
| 61 | 7-13-6-1 | −5.97 | −2.49 | 2.64 | 2.35 | 2.88 | 0.367 | 0.29 | −0.24 | 4 |
| 62 | 7-10-13-6-3 | −6.41 | −2.89 | 2.73 | 2.35 | 2.94 | 0.456 | 0.37 | −0.21 | 5 |
| 63 | 7-4 | −5.78 | −2.27 | 2.70 | 2.35 | 2.89 | 0.479 | 0.35 | −0.19 | 2 |
| 64 | 7-8-6-2 | −6.26 | −2.72 | 2.67 | 2.35 | 2.91 | 0.224 | 0.31 | −0.24 | 4 |
| 65 | 7-13-6-4 | −6.07 | −2.57 | 2.67 | 2.35 | 2.88 | 0.380 | 0.32 | −0.21 | 4 |
| 66 | 7-10-13-6-1-3 | −6.50 | −3.01 | 2.69 | 2.35 | 2.86 | 0.453 | 0.34 | −0.17 | 6 |
| 67 | 7-8-12-6-4 | −6.45 | −2.92 | 2.67 | 2.35 | 2.87 | 0.261 | 0.32 | −0.20 | 5 |
| 68 | 7-11-5-3 | −6.53 | −2.96 | 2.78 | 2.35 | 2.87 | 0.535 | 0.43 | −0.09 | 4 |
| 69 | 7-6-4 | −5.99 | −2.52 | 2.63 | 2.35 | 2.89 | 0.318 | 0.28 | −0.26 | 3 |
| 70 | 7-12-6-4 | −6.24 | −2.73 | 2.66 | 2.35 | 2.91 | 0.314 | 0.32 | −0.24 | 4 |
| 71 | 7-8-13-6-2 | −6.34 | −2.82 | 2.67 | 2.34 | 2.85 | 0.278 | 0.32 | −0.19 | 5 |
| 72 | 7-10-12-6 | −6.25 | −2.71 | 2.70 | 2.34 | 2.93 | 0.344 | 0.36 | −0.23 | 4 |
| 73 | 7-10-6-4 | −6.17 | −2.69 | 2.64 | 2.34 | 2.89 | 0.352 | 0.30 | −0.25 | 4 |
| 74 | 7-2 | −5.84 | −2.32 | 2.70 | 2.34 | 2.88 | 0.396 | 0.36 | −0.18 | 2 |
| 75 | 7-12-1-3 | −6.23 | −2.69 | 2.75 | 2.34 | 2.97 | 0.457 | 0.41 | −0.22 | 4 |
| 76 | 7-13-1 | −5.76 | −2.27 | 2.69 | 2.34 | 2.89 | 0.522 | 0.35 | −0.19 | 3 |
| 77 | 7-11-13-2-4 | −6.43 | −2.90 | 2.74 | 2.34 | 2.93 | 0.475 | 0.40 | −0.19 | 5 |
| 78 | 7-1 | −5.69 | −2.18 | 2.70 | 2.34 | 2.91 | 0.486 | 0.36 | −0.21 | 2 |
| 79 | 7-8-13-6-2-4 | −6.56 | −3.05 | 2.65 | 2.34 | 2.79 | 0.278 | 0.32 | −0.13 | 6 |
| 80 | 7-11-13-6-2 | −6.43 | −2.90 | 2.70 | 2.34 | 2.89 | 0.298 | 0.36 | −0.19 | 5 |
| 81 | 7-10-12-6-1 | −6.33 | −2.84 | 2.66 | 2.34 | 2.88 | 0.343 | 0.32 | −0.22 | 5 |
| 82 | 7-10-6-1 | −6.07 | −2.61 | 2.62 | 2.34 | 2.91 | 0.340 | 0.28 | −0.29 | 4 |
| 83 | 7-9-11-6 | −6.42 | −2.86 | 2.74 | 2.33 | 2.90 | 0.399 | 0.41 | −0.16 | 4 |
| 84 | 7-10-4 | −5.95 | −2.47 | 2.68 | 2.33 | 2.90 | 0.490 | 0.35 | −0.22 | 3 |
| 85 | 7-10-12-6-4 | −6.44 | −2.93 | 2.68 | 2.33 | 2.89 | 0.349 | 0.34 | −0.21 | 5 |
| 86 | 7-12-6-1 | −6.13 | −2.65 | 2.64 | 2.33 | 2.91 | 0.304 | 0.30 | −0.28 | 4 |
| 87 | 7-13-4 | −5.85 | −2.37 | 2.69 | 2.33 | 2.90 | 0.504 | 0.36 | −0.21 | 3 |
| 88 | 7-9-11-6-1 | −6.50 | −2.99 | 2.68 | 2.33 | 2.85 | 0.385 | 0.35 | −0.17 | 5 |
| 89 | 7-6-1 | −5.90 | −2.45 | 2.60 | 2.33 | 2.91 | 0.305 | 0.27 | −0.31 | 3 |
| 90 | 7-12-4 | −6.01 | −2.51 | 2.69 | 2.33 | 2.90 | 0.416 | 0.36 | −0.21 | 3 |
| 91 | 7-11-13-5-3 | −6.59 | −3.06 | 2.76 | 2.33 | 2.84 | 0.557 | 0.43 | −0.08 | 5 |
| 92 | 7-8-10-6-2-4 | −6.67 | −3.17 | 2.64 | 2.32 | 2.79 | 0.256 | 0.31 | −0.16 | 6 |
| 93 | 7-13-2 | −5.91 | −2.41 | 2.70 | 2.32 | 2.90 | 0.433 | 0.37 | −0.20 | 3 |
| 94 | 7-9-11-6-4 | −6.60 | −3.08 | 2.70 | 2.32 | 2.86 | 0.395 | 0.38 | −0.16 | 5 |
| 95 | 7-12-2 | −6.08 | −2.56 | 2.70 | 2.32 | 2.90 | 0.353 | 0.38 | −0.20 | 3 |
| 96 | 7-11-6-2-4 | −6.56 | −3.10 | 2.60 | 2.32 | 2.80 | 0.239 | 0.29 | −0.19 | 5 |
| 97 | 7-8-12-6-2 | −6.48 | −2.97 | 2.65 | 2.31 | 2.81 | 0.213 | 0.33 | −0.16 | 5 |
| 98 | 7-8-6-2-4 | −6.47 | −2.99 | 2.61 | 2.31 | 2.81 | 0.216 | 0.30 | −0.19 | 5 |
| 99 | 7-11-13-1-4 | −6.27 | −2.81 | 2.69 | 2.31 | 2.87 | 0.546 | 0.38 | −0.18 | 5 |
| 100 | 7-9-11-13-6-1 | −6.59 | −3.08 | 2.61 | 2.31 | 2.80 | 0.000 | 0.30 | −0.19 | 6 |
| 101 | 7-8-13-6-1-4 | −6.41 | −2.97 | 2.60 | 2.31 | 2.73 | 0.338 | 0.29 | −0.13 | 6 |
| 102 | 7-13-6-2 | −6.10 | −2.63 | 2.63 | 2.31 | 2.84 | 0.267 | 0.32 | −0.21 | 4 |
| 103 | 7-13-2-4 | −6.10 | −2.62 | 2.68 | 2.31 | 2.87 | 0.438 | 0.37 | −0.19 | 4 |
| 104 | 7-11-1-4 | −6.20 | −2.73 | 2.69 | 2.31 | 2.90 | 0.508 | 0.38 | −0.21 | 4 |
| 105 | 7-11-13-6-1-4 | −6.50 | −3.08 | 2.60 | 2.31 | 2.71 | 0.362 | 0.29 | −0.11 | 6 |
| 106 | 7-11-5-1-3 | −6.62 | −3.08 | 2.69 | 2.31 | 2.86 | 0.000 | 0.38 | −0.17 | 5 |
| 107 | 7-12-6-2 | −6.28 | −2.79 | 2.62 | 2.30 | 2.87 | 0.218 | 0.32 | −0.25 | 4 |
| 108 | 7-9-6-3 | −6.37 | −2.87 | 2.69 | 2.30 | 2.84 | 0.398 | 0.39 | −0.15 | 4 |
| 109 | 7-8-10-12-6-2-4 | −6.92 | −3.43 | 2.64 | 2.30 | 2.76 | 0.249 | 0.34 | −0.12 | 7 |
| 110 | 7-10-6-2 | −6.21 | −2.75 | 2.60 | 2.30 | 2.86 | 0.243 | 0.30 | −0.26 | 4 |
| 111 | 7-9-13-6-3 | −6.48 | −2.96 | 2.72 | 2.30 | 2.97 | 0.451 | 0.42 | −0.25 | 5 |
| 112 | 7-10-13-6-1 | −6.17 | −2.73 | 2.63 | 2.30 | 2.83 | 0.410 | 0.33 | −0.20 | 5 |
| 113 | 7-6-2 | −6.03 | −2.58 | 2.58 | 2.30 | 2.88 | 0.211 | 0.28 | −0.30 | 3 |
| 114 | 7-2-4 | −6.03 | −2.56 | 2.67 | 2.30 | 2.89 | 0.388 | 0.37 | −0.22 | 3 |
| 115 | 7-13-5-3 | −6.27 | −2.77 | 2.71 | 2.30 | 2.86 | 0.520 | 0.41 | −0.15 | 4 |
| 116 | 7-8-12-6-2-4 | −6.70 | −3.21 | 2.63 | 2.30 | 2.78 | 0.221 | 0.33 | −0.16 | 6 |
| 117 | 7-10-12-6-2 | −6.48 | −2.99 | 2.64 | 2.30 | 2.84 | 0.255 | 0.35 | −0.20 | 5 |
| 118 | 7-9-10-6-3 | −6.58 | −3.07 | 2.71 | 2.30 | 2.97 | 0.436 | 0.41 | −0.26 | 5 |
| 119 | 7-12-2-4 | −6.27 | −2.78 | 2.68 | 2.30 | 2.90 | 0.362 | 0.38 | −0.22 | 4 |
| 120 | 7-10-2-4 | −6.21 | −2.74 | 2.67 | 2.30 | 2.89 | 0.417 | 0.37 | −0.23 | 4 |
| 121 | 7-5-3 | −6.20 | −2.69 | 2.72 | 2.29 | 2.87 | 0.489 | 0.43 | −0.15 | 3 |
| 122 | 7-9-11-13-6-4 | −6.70 | −3.17 | 2.68 | 2.29 | 2.86 | 0.000 | 0.39 | −0.17 | 6 |
| 123 | 7-10-12-2-4 | −6.47 | −2.99 | 2.68 | 2.29 | 2.87 | 0.384 | 0.39 | −0.19 | 5 |
| 124 | 7-10-5-3 | −6.38 | −2.88 | 2.71 | 2.29 | 2.88 | 0.506 | 0.42 | −0.17 | 4 |
| 125 | 7-8-10-6-1-4 | −6.52 | −3.09 | 2.59 | 2.29 | 2.77 | 0.320 | 0.31 | −0.18 | 6 |
| 126 | 7-9-11-13-6 | −6.50 | −2.96 | 2.73 | 2.28 | 2.90 | 0.000 | 0.45 | −0.16 | 5 |
| 127 | 7-9-13-6-1-3 | −6.54 | −3.09 | 2.66 | 2.28 | 2.89 | 0.443 | 0.38 | −0.23 | 6 |
| 128 | 7-9-11-6-2 | −6.64 | −3.14 | 2.66 | 2.28 | 2.83 | 0.288 | 0.38 | −0.16 | 5 |
| 129 | 7-11-6-1-4 | −6.43 | −3.03 | 2.56 | 2.28 | 2.77 | 0.320 | 0.28 | −0.20 | 5 |
| 130 | 7-12-5-3 | −6.44 | −2.93 | 2.72 | 2.28 | 2.87 | 0.430 | 0.44 | −0.15 | 4 |
| 131 | 7-10-13-6 | −6.08 | −2.63 | 2.66 | 2.28 | 2.89 | 0.413 | 0.38 | −0.23 | 4 |
| 132 | 7-10-13-6-4 | −6.26 | −2.82 | 2.64 | 2.28 | 2.84 | 0.415 | 0.36 | −0.20 | 5 |
| 133 | 7-13-6-2-4 | −6.31 | −2.88 | 2.57 | 2.28 | 2.81 | 0.251 | 0.29 | −0.24 | 5 |
| 134 | 7-8-10-13-6-2-4 | −6.76 | −3.33 | 2.60 | 2.28 | 2.73 | 0.306 | 0.33 | −0.13 | 7 |
| 135 | 7-10-12-6-2-4 | −6.69 | −3.24 | 2.60 | 2.27 | 2.78 | 0.247 | 0.32 | −0.19 | 6 |
| 136 | 7-10-12-5-3 | −6.64 | −3.14 | 2.71 | 2.27 | 2.84 | 0.446 | 0.43 | −0.14 | 5 |
| 137 | 7-9-12-6-3 | −6.63 | −3.12 | 2.69 | 2.27 | 2.95 | 0.337 | 0.42 | −0.26 | 5 |
| 138 | 7-8-10-13-6-1-4 | −6.61 | −3.22 | 2.57 | 2.27 | 2.68 | 0.360 | 0.30 | −0.11 | 7 |
| 139 | 7-9-10-12-6-3 | −6.86 | −3.34 | 2.58 | 2.27 | 2.74 | 0.000 | 0.32 | −0.16 | 6 |
| 140 | 7-11-5 | −6.16 | −2.69 | 2.68 | 2.27 | 2.84 | 0.478 | 0.42 | −0.15 | 3 |
| 141 | 7-13-5-1-3 | −6.36 | −2.89 | 2.65 | 2.27 | 2.83 | 0.000 | 0.38 | −0.18 | 5 |
| 142 | 7-8-6-1-4 | −6.32 | −2.92 | 2.56 | 2.27 | 2.78 | 0.281 | 0.30 | −0.22 | 5 |
| 143 | 7-13-1-4 | −5.95 | −2.54 | 2.63 | 2.27 | 2.85 | 0.506 | 0.36 | −0.22 | 4 |
| 144 | 7-9-11-6-2-4 | −6.85 | −3.40 | 2.61 | 2.27 | 2.76 | 0.279 | 0.35 | −0.15 | 6 |
| 145 | 7-12-6-2-4 | −6.48 | −3.06 | 2.56 | 2.26 | 2.80 | 0.211 | 0.30 | −0.24 | 5 |
| 146 | 7-9-6 | −6.06 | −2.60 | 2.64 | 2.26 | 2.85 | 0.367 | 0.38 | −0.20 | 3 |
| 147 | 7-8-12-6-1-4 | −6.54 | −3.13 | 2.58 | 2.26 | 2.76 | 0.279 | 0.32 | −0.18 | 6 |
| 148 | 7-9-10-6-1-3 | −6.64 | −3.20 | 2.65 | 2.26 | 2.92 | 0.421 | 0.39 | −0.27 | 6 |
| 149 | 7-9-11-13-6-2-4 | −6.94 | −3.46 | 2.58 | 2.26 | 2.73 | 0.000 | 0.32 | −0.15 | 7 |
| 150 | 7-11-5-1 | −6.26 | −2.79 | 2.69 | 2.25 | 2.90 | 0.508 | 0.44 | −0.21 | 4 |
| 151 | 7-10-6-2-4 | −6.41 | −3.02 | 2.53 | 2.25 | 2.81 | 0.233 | 0.28 | −0.28 | 5 |
| 152 | 7-10-13-6-2 | −6.30 | −2.87 | 2.61 | 2.25 | 2.78 | 0.320 | 0.36 | −0.17 | 5 |
| 153 | 7-9-11-5-3 | −6.80 | −3.31 | 2.73 | 2.25 | 2.80 | 0.526 | 0.47 | −0.07 | 5 |
| 154 | 7-9-11-13-6-1-4 | −6.78 | −3.38 | 2.51 | 2.25 | 2.66 | 0.000 | 0.25 | −0.16 | 7 |
| 155 | 7-11-5-4 | −6.35 | −2.89 | 2.68 | 2.25 | 2.89 | 0.501 | 0.43 | −0.21 | 4 |
| 156 | 7-9-11-13-6-2 | −6.72 | −3.22 | 2.66 | 2.25 | 2.83 | 0.000 | 0.40 | −0.18 | 6 |
| 157 | 7-9-10-6 | −6.25 | −2.79 | 2.65 | 2.25 | 2.92 | 0.395 | 0.40 | −0.26 | 4 |
| 158 | 7-9-10-12-6-1-3 | −6.93 | −3.46 | 2.51 | 2.25 | 2.69 | 0.000 | 0.26 | −0.18 | 7 |
| 159 | 7-9-13-6-1 | −6.22 | −2.80 | 2.61 | 2.25 | 2.85 | 0.401 | 0.37 | −0.23 | 5 |
| 160 | 7-11-13-5-1 | −6.32 | −2.88 | 2.67 | 2.25 | 2.90 | 0.541 | 0.43 | −0.23 | 5 |
| 161 | 7-9-6-1-3 | −6.43 | −3.02 | 2.61 | 2.25 | 2.81 | 0.371 | 0.36 | −0.20 | 5 |
| 162 | 7-10-12-5-1-3 | −6.72 | −3.24 | 2.64 | 2.24 | 2.83 | 0.000 | 0.40 | −0.19 | 6 |
| 163 | 7-13-6-1-4 | −6.16 | −2.82 | 2.52 | 2.24 | 2.74 | 0.324 | 0.27 | −0.22 | 5 |
| 164 | 7-10-5-1-3 | −6.46 | −2.99 | 2.70 | 2.24 | 2.89 | 0.518 | 0.46 | −0.19 | 5 |
| 165 | 7-6-2-4 | −6.23 | −2.86 | 2.51 | 2.24 | 2.83 | 0.206 | 0.26 | −0.32 | 4 |
| 166 | 7-10-13-2-4 | −6.29 | −2.89 | 2.63 | 2.24 | 2.83 | 0.448 | 0.39 | −0.20 | 5 |
| 167 | 7-12-1-4 | −6.11 | −2.70 | 2.63 | 2.24 | 2.87 | 0.426 | 0.39 | −0.24 | 4 |
| 168 | 7-9-10-13-6-3 | −6.70 | −3.22 | 2.54 | 2.24 | 2.63 | 0.000 | 0.30 | −0.09 | 6 |
| 169 | 7-10-12-6-1-4 | −6.53 | −3.16 | 2.55 | 2.24 | 2.76 | 0.312 | 0.31 | −0.21 | 6 |
| 170 | 7-9-13-6 | −6.15 | −2.69 | 2.67 | 2.24 | 2.92 | 0.410 | 0.43 | −0.25 | 4 |
| 171 | 7-9-6-4 | −6.23 | −2.82 | 2.59 | 2.24 | 2.82 | 0.351 | 0.35 | −0.22 | 4 |
| 172 | 7-9-13-6-4 | −6.33 | −2.89 | 2.64 | 2.24 | 2.87 | 0.408 | 0.40 | −0.23 | 5 |
| 173 | 7-9-11-6-1-4 | −6.69 | −3.32 | 2.56 | 2.24 | 2.74 | 0.346 | 0.32 | −0.18 | 6 |
| 174 | 7-10-1-4 | −6.04 | −2.65 | 2.62 | 2.24 | 2.88 | 0.481 | 0.38 | −0.26 | 4 |
| 175 | 7-9-10-6-4 | −6.43 | −3.01 | 2.62 | 2.24 | 2.86 | 0.386 | 0.38 | −0.25 | 5 |
| 176 | 7-10-13-5-3 | −6.46 | −3.05 | 2.65 | 2.24 | 2.82 | 0.508 | 0.41 | −0.17 | 5 |
| 177 | 7-1-4 | −5.88 | −2.49 | 2.61 | 2.24 | 2.90 | 0.457 | 0.38 | −0.28 | 3 |
| 178 | 7-5-1-3 | −6.29 | −2.81 | 2.70 | 2.24 | 2.86 | 0.487 | 0.47 | −0.16 | 4 |
| 179 | 7-9-10-6-1 | −6.32 | −2.92 | 2.59 | 2.24 | 2.84 | 0.378 | 0.36 | −0.25 | 5 |
| 180 | 7-12-5-1-3 | −6.52 | −3.04 | 2.70 | 2.23 | 2.87 | 0.000 | 0.46 | −0.18 | 5 |
| 181 | 7-5 | −5.86 | −2.42 | 2.64 | 2.23 | 2.84 | 0.451 | 0.41 | −0.20 | 2 |
| 182 | 7-11-13-5-4 | −6.41 | −2.98 | 2.67 | 2.23 | 2.90 | 0.529 | 0.44 | −0.23 | 5 |
| 183 | 7-11-13-5 | −6.21 | −2.79 | 2.66 | 2.23 | 2.83 | 0.494 | 0.43 | −0.17 | 4 |
| 184 | 7-9-6-1 | −6.12 | −2.74 | 2.56 | 2.23 | 2.81 | 0.340 | 0.33 | −0.25 | 4 |
| 185 | 7-10-13-5-1-3 | −6.55 | −3.14 | 2.57 | 2.23 | 2.75 | 0.000 | 0.34 | −0.18 | 6 |
| 186 | 7-9-11-5-1-3 | −6.89 | −3.42 | 2.57 | 2.23 | 2.76 | 0.000 | 0.34 | −0.19 | 6 |
| 187 | 7-9-12-6-1-3 | −6.68 | −3.25 | 2.63 | 2.23 | 2.93 | 0.337 | 0.40 | −0.30 | 6 |
| 188 | 7-9-12-6 | −6.31 | −2.85 | 2.64 | 2.23 | 2.93 | 0.321 | 0.42 | −0.28 | 4 |
| 189 | 7-11-5-2 | −6.41 | −2.95 | 2.66 | 2.22 | 2.89 | 0.382 | 0.43 | −0.23 | 4 |
| 190 | 7-10-13-1-4 | −6.14 | −2.79 | 2.59 | 2.22 | 2.78 | 0.509 | 0.37 | −0.19 | 5 |
| 191 | 7-12-6-1-4 | −6.33 | −2.99 | 2.51 | 2.22 | 2.78 | 0.277 | 0.29 | −0.26 | 5 |
| 192 | 7-10-5 | −6.02 | −2.62 | 2.62 | 2.22 | 2.83 | 0.461 | 0.40 | −0.21 | 3 |
| 193 | 7-13-5 | −5.91 | −2.51 | 2.62 | 2.22 | 2.82 | 0.472 | 0.40 | −0.20 | 3 |
| 194 | 7-9-12-6-4 | −6.49 | −3.06 | 2.61 | 2.22 | 2.88 | 0.322 | 0.40 | −0.27 | 5 |
| 195 | 7-13-5-1 | −6.00 | −2.60 | 2.63 | 2.22 | 2.87 | 0.506 | 0.41 | −0.24 | 4 |
| 196 | 7-9-10-12-6-1 | −6.59 | −3.16 | 2.58 | 2.22 | 2.75 | 0.000 | 0.36 | −0.17 | 6 |
| 197 | 7-12-5 | −6.08 | −2.66 | 2.63 | 2.21 | 2.82 | 0.401 | 0.42 | −0.19 | 3 |
| 198 | 7-11-5-2-4 | −6.62 | −3.18 | 2.64 | 2.21 | 2.79 | 0.000 | 0.42 | −0.15 | 5 |
| 199 | 7-9-10-12-6-4 | −6.71 | −3.27 | 2.61 | 2.21 | 2.77 | 0.000 | 0.40 | −0.16 | 6 |
| 200 | 7-13-5-4 | −6.10 | −2.70 | 2.63 | 2.21 | 2.86 | 0.495 | 0.41 | −0.23 | 4 |
| 201 | 7-9-10-12-6 | −6.52 | −3.07 | 2.65 | 2.21 | 2.80 | 0.000 | 0.44 | −0.14 | 5 |
| 202 | 7-9-13-6-2 | −6.37 | −2.95 | 2.61 | 2.21 | 2.80 | 0.317 | 0.40 | −0.19 | 5 |
| 203 | 7-9-6-2 | −6.27 | −2.88 | 2.56 | 2.21 | 2.78 | 0.258 | 0.35 | −0.23 | 4 |
| 204 | 7-9-12-6-1 | −6.36 | −2.96 | 2.58 | 2.21 | 2.85 | 0.320 | 0.38 | −0.27 | 5 |
| 205 | 7-9-10-6-2 | −6.47 | −3.07 | 2.58 | 2.21 | 2.81 | 0.294 | 0.38 | −0.22 | 5 |
| 206 | 7-5-4 | −6.04 | −2.64 | 2.62 | 2.20 | 2.88 | 0.458 | 0.42 | −0.26 | 3 |
| 207 | 7-10-5-4 | −6.21 | −2.82 | 2.62 | 2.20 | 2.89 | 0.477 | 0.42 | −0.27 | 4 |
| 208 | 7-12-5-4 | −6.28 | −2.86 | 2.63 | 2.20 | 2.89 | 0.421 | 0.43 | −0.25 | 4 |
| 209 | 7-9-10-13-6-1 | −6.43 | −3.04 | 2.50 | 2.20 | 2.60 | 0.000 | 0.30 | −0.10 | 6 |
| 210 | 7-9-13-6-2-4 | −6.57 | −3.19 | 2.56 | 2.20 | 2.77 | 0.295 | 0.36 | −0.21 | 6 |
| 211 | 7-10-5-1 | −6.12 | −2.71 | 2.63 | 2.20 | 2.91 | 0.486 | 0.43 | −0.28 | 4 |
| 212 | 7-5-1 | −5.95 | −2.54 | 2.63 | 2.20 | 2.89 | 0.463 | 0.43 | −0.26 | 3 |
| 213 | 7-11-13-5-2 | −6.46 | −3.04 | 2.64 | 2.20 | 2.91 | 0.408 | 0.44 | −0.27 | 5 |
| 214 | 7-12-5-1 | −6.17 | −2.76 | 2.63 | 2.19 | 2.90 | 0.427 | 0.44 | −0.27 | 4 |
| 215 | 7-10-12-5-1 | −6.37 | −2.96 | 2.63 | 2.19 | 2.86 | 0.445 | 0.43 | −0.23 | 5 |
| 216 | 7-10-12-5 | −6.27 | −2.88 | 2.60 | 2.19 | 2.80 | 0.401 | 0.41 | −0.20 | 4 |
| 217 | 7-10-12-5-4 | −6.47 | −3.07 | 2.62 | 2.19 | 2.85 | 0.431 | 0.43 | −0.23 | 5 |
| 218 | 7-9-10-12-6-2 | −6.75 | −3.32 | 2.60 | 2.19 | 2.76 | 0.000 | 0.41 | −0.16 | 6 |
| 219 | 7-9-12-6-2 | −6.52 | −3.12 | 2.58 | 2.19 | 2.82 | 0.252 | 0.40 | −0.23 | 5 |
| 220 | 7-11-5-1-4 | −6.47 | −3.07 | 2.57 | 2.19 | 2.65 | 0.000 | 0.38 | −0.08 | 5 |
| 221 | 7-9-10-13-6-4 | −6.55 | −3.15 | 2.57 | 2.19 | 2.65 | 0.000 | 0.38 | −0.09 | 6 |
| 222 | 7-9-10-6-2-4 | −6.67 | −3.32 | 2.53 | 2.18 | 2.77 | 0.274 | 0.35 | −0.24 | 6 |
| 223 | 7-9-10-12-6-2-4 | −6.96 | −3.56 | 2.54 | 2.18 | 2.71 | 0.000 | 0.35 | −0.18 | 7 |
| 224 | 7-9-13-5-3 | −6.53 | −3.13 | 2.65 | 2.18 | 2.81 | 0.510 | 0.47 | −0.17 | 5 |
| 225 | 7-5-2 | −6.09 | −2.69 | 2.61 | 2.18 | 2.87 | 0.359 | 0.43 | −0.26 | 3 |
| 226 | 7-13-5-2 | −6.15 | −2.76 | 2.60 | 2.18 | 2.86 | 0.394 | 0.42 | −0.26 | 4 |
| 227 | 7-9-13-6-1-4 | −6.41 | −3.11 | 2.50 | 2.18 | 2.70 | 0.354 | 0.33 | −0.20 | 6 |
| 228 | 7-9-10-13-6 | −6.36 | −2.96 | 2.60 | 2.18 | 2.67 | 0.000 | 0.42 | −0.08 | 5 |
| 229 | 7-10-5-2 | −6.27 | −2.87 | 2.60 | 2.18 | 2.90 | 0.378 | 0.42 | −0.29 | 4 |
| 230 | 7-9-5-3 | −6.44 | −3.04 | 2.64 | 2.17 | 2.79 | 0.471 | 0.46 | −0.15 | 4 |
| 231 | 7-12-5-2 | −6.33 | −2.92 | 2.61 | 2.17 | 2.89 | 0.333 | 0.44 | −0.28 | 4 |
| 232 | 7-13-5-2-4 | −6.36 | −2.97 | 2.61 | 2.17 | 2.79 | 0.415 | 0.44 | −0.18 | 5 |
| 233 | 7-9-11-5-4 | −6.63 | −3.24 | 2.64 | 2.17 | 2.82 | 0.501 | 0.47 | −0.18 | 5 |
| 234 | 7-10-13-5-1 | −6.20 | −2.86 | 2.58 | 2.17 | 2.80 | 0.502 | 0.41 | −0.22 | 5 |
| 235 | 7-9-12-6-2-4 | −6.73 | −3.36 | 2.53 | 2.17 | 2.77 | 0.239 | 0.36 | −0.24 | 6 |
| 236 | 7-9-13-5-1-3 | −6.61 | −3.23 | 2.52 | 2.17 | 2.71 | 0.000 | 0.35 | −0.20 | 6 |
| 237 | 7-10-12-5-2 | −6.52 | −3.13 | 2.60 | 2.17 | 2.86 | 0.346 | 0.43 | −0.26 | 5 |
| 238 | 7-9-10-13-6-2-4 | −6.79 | −3.42 | 2.50 | 2.17 | 2.60 | 0.000 | 0.33 | −0.10 | 7 |
| 239 | 7-10-12-5-2-4 | −6.74 | −3.34 | 2.60 | 2.16 | 2.76 | 0.000 | 0.44 | −0.16 | 6 |
| 240 | 7-12-5-2-4 | −6.54 | −3.14 | 2.61 | 2.16 | 2.79 | 0.348 | 0.45 | −0.18 | 5 |
| 241 | 7-9-10-13-6-2 | −6.58 | −3.20 | 2.55 | 2.16 | 2.64 | 0.000 | 0.39 | −0.09 | 6 |
| 242 | 7-10-5-2-4 | −6.48 | −3.10 | 2.60 | 2.16 | 2.81 | 0.395 | 0.44 | −0.21 | 5 |
| 243 | 7-9-10-12-6-1-4 | −6.79 | −3.46 | 2.50 | 2.16 | 2.68 | 0.000 | 0.34 | −0.18 | 7 |
| 244 | 7-13-5-1-4 | −6.21 | −2.87 | 2.53 | 2.16 | 2.63 | 0.000 | 0.38 | −0.10 | 5 |
| 245 | 7-5-2-4 | −6.30 | −2.92 | 2.59 | 2.16 | 2.84 | 0.368 | 0.44 | −0.24 | 4 |
| 246 | 7-10-13-5-4 | −6.29 | −2.97 | 2.57 | 2.15 | 2.80 | 0.488 | 0.42 | −0.23 | 5 |
| 247 | 7-10-13-5 | −6.10 | −2.79 | 2.55 | 2.15 | 2.79 | 0.457 | 0.40 | −0.24 | 4 |
| 248 | 7-9-11-5-2-4 | −6.91 | −3.51 | 2.55 | 2.15 | 2.72 | 0.000 | 0.40 | −0.17 | 6 |
| 249 | 7-9-12-5-3 | −6.69 | −3.30 | 2.62 | 2.15 | 2.81 | 0.394 | 0.47 | −0.19 | 5 |
| 250 | 7-9-11-13-5-4 | −6.71 | −3.34 | 2.60 | 2.14 | 2.78 | 0.000 | 0.45 | −0.18 | 6 |
| 251 | 7-10-12-5-1-4 | −6.58 | −3.22 | 2.54 | 2.14 | 2.63 | 0.000 | 0.39 | −0.09 | 6 |
| 252 | 7-10-13-5-2-4 | −6.56 | −3.22 | 2.56 | 2.14 | 2.73 | 0.000 | 0.43 | −0.17 | 6 |
| 253 | 7-9-5-1-3 | −6.50 | −3.14 | 2.62 | 2.13 | 2.78 | 0.476 | 0.48 | −0.16 | 5 |
| 254 | 7-10-5-1-4 | −6.32 | −2.99 | 2.58 | 2.13 | 2.68 | 0.473 | 0.45 | −0.09 | 5 |
| 255 | 7-12-5-1-4 | −6.38 | −3.03 | 2.58 | 2.13 | 2.66 | 0.000 | 0.44 | −0.08 | 5 |
| 256 | 7-10-13-5-2 | −6.34 | −3.02 | 2.55 | 2.13 | 2.80 | 0.402 | 0.42 | −0.24 | 5 |
| 257 | 7-5-1-4 | −6.15 | −2.82 | 2.57 | 2.12 | 2.70 | 0.445 | 0.45 | −0.12 | 4 |
| 258 | 7-9-13-5-1 | −6.26 | −2.94 | 2.58 | 2.12 | 2.81 | 0.506 | 0.45 | −0.24 | 5 |
| 259 | 7-9-5 | −6.10 | −2.77 | 2.56 | 2.12 | 2.77 | 0.445 | 0.45 | −0.21 | 3 |
| 260 | 7-9-12-5-1-3 | −6.76 | −3.39 | 2.58 | 2.12 | 2.78 | 0.000 | 0.47 | −0.19 | 6 |
| 261 | 7-9-5-4 | −6.28 | −2.96 | 2.56 | 2.11 | 2.79 | 0.458 | 0.45 | −0.23 | 4 |
| 262 | 7-9-13-5-4 | −6.37 | −3.05 | 2.57 | 2.11 | 2.83 | 0.491 | 0.46 | −0.25 | 5 |
| 263 | 7-9-5-1 | −6.17 | −2.85 | 2.56 | 2.10 | 2.80 | 0.465 | 0.46 | −0.24 | 4 |
| 264 | 7-9-10-5-4 | −6.48 | −3.16 | 2.57 | 2.10 | 2.84 | 0.478 | 0.46 | −0.27 | 5. |

Developing blue fluorescent (fl) dopants using aza-substituted DiPYR core with high quantum efficiencies and small singlet-triplet energy gap (ΔS₁/T₁<0.4 eV).

By screening a library of blue fluorescent emitters and estimating their respective S₁, T₁, HOMO, and LUMO energies, the best candidates of emitters suitable for a hybrid WOLED can be identified. Using this information, promising candidates acting as blue fl dopants with high quantum efficiencies and small singlet-triplet energy gap (ΔS₁/T₁<0.4 eV) have been successfully synthesized and characterized. Their photophysical and electrochemical properties in both solution and solid matrices have been analyzed as presented herein. This combined theoretical/experimental approach allowed us to rapidly target potential candidates that matches the criteria of hybrid blue fluorescent (“fl”) emitters.

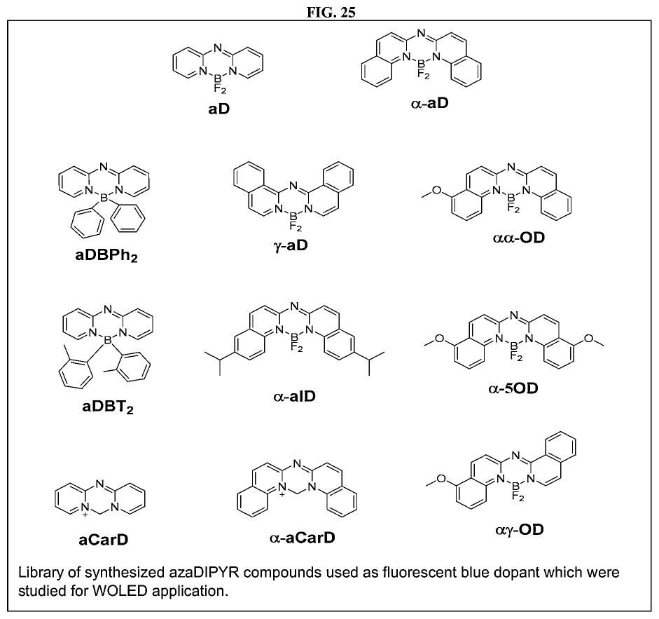

In the present work, our efforts were focused on synthesizing derivatives of difluoro-boron-azaDiPYR complexes. Boyer and coworkers first discovered these complexes in the early nineties, but little information is known about their application in OLED devices. Recently, new synthetic procedures of aza-substituted DiPYR complexes emerged inspiring a new family of dyes to investigate. The target complexes acting as blue emitters are shown in FIG. 25. These dyes strongly absorb in the UV-blue part of the spectrum and display blue fluorescence (λₑₘ=400-470 nm) with high quantum yields in solution and when doped in thin films (Φ=42%-86%).

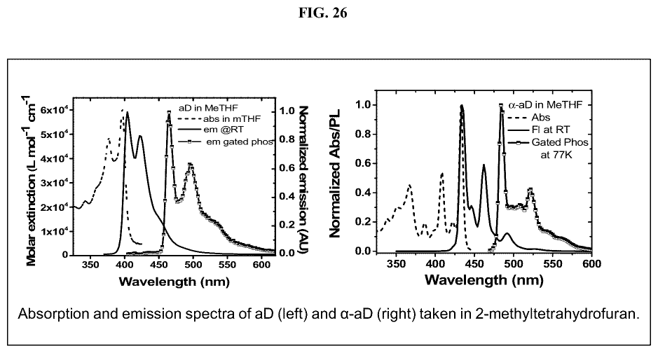

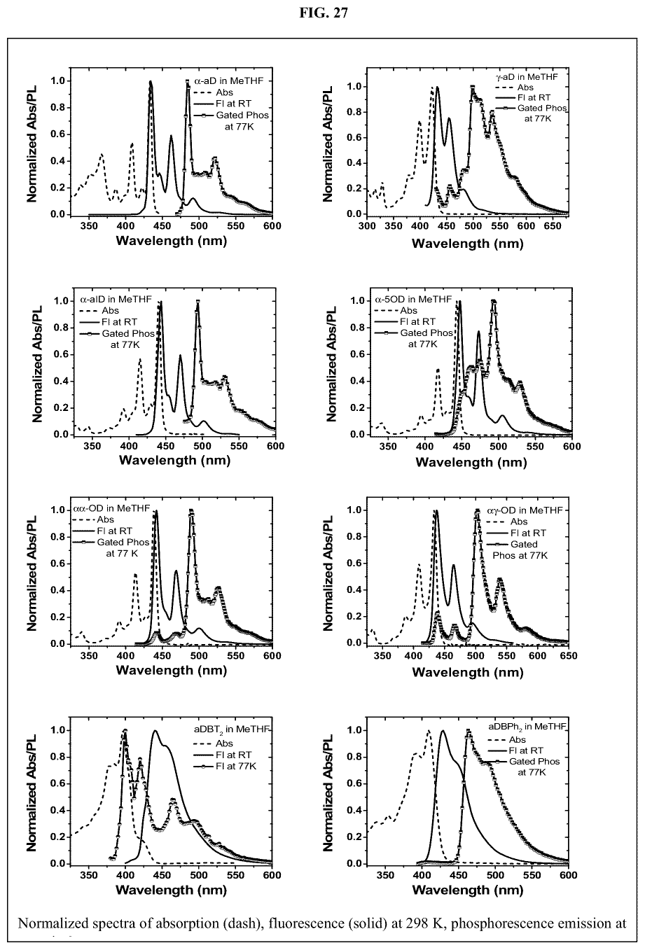

The absorption and emission spectra of the parent aD and the benzannulated derivatives such as α-aD and others are shown in FIG. 26. These complexes exhibit strong π to π* absorption bands and intense fluorescence emission in the visible spectrum. The parent aD complex has violet-blue fluorescence emission (λ_{em, max}=405 nm) that is the mirror image of the S₀ to S₁ transition (λ_{abs, max}=398 nm). In the UV region of the absorption spectra, the S₀ to S₂ transition centered at 270 nm also appears as a vibrational absorption feature. For the benzannulated derivatives such as α-aD, both the absorption and emission profile are red shifted compared to the parent aD. This information increases the number of potential candidates of Host materials with high HOMO and low LUMO energies that can nest the latter blue emitter complex. The gated phosphorescence emission (shown in half-filled boxed lines in FIG. 27 taken at low temperatures (77 K) after 500 μs delay time represents the lowest triplet energy (T₁).

Table 6 compares the singlet and triplet energies for the respective complexes from the calculated methods to the experimental data. Clearly, the computational data of the singlet-triplet energy gap closely matches the experimental data. The α-aD has a small singlet-triplet energy gap of ΔS₁/T₁≤0.3 eV that meets the criteria of developing blue fl dopants for hybrid WOLED, and the parent aD has a singlet-triplet energy gap of ΔS₁/T₁≤0.5 eV. The HOMO and LUMO energies recorded in Table 7 were derived from electrochemical data using Ferrocene as an internal standard in acetonitrile solvent. From the DFT calculations, we were able to understand that the higher quantum yield of α-aD (Φ_{f}=86%) in solution was a result of the relative reordering of the S₁ and T₂ energies upon benzannulation of the parent structure. The reason the parent aD has a lower quantum yield (Φ_{f}=42%) is because the energy of T₂ states is just below the S₁ state, enabling fast intersystem crossing that is competitive with fluorescence. All photophysical parameters for the unsubstituted DiPYR derivative aD and the benzannulated derivatives like α-aD complexes present in solution and doped in 1 wt % in PMMA films are summarized in Table 8. Note that the quantum efficiency of α-aD remains high in both solution and solid state (Φ_{f}=86%), maintaining a small singlet-triplet energy gap necessary for blue fl emitter in the hybrid WOLED.

The experimental HOMO energies of γ-aD (6.14 eV) and α-aD (6.21 eV) deviated from the calculated HOMO energies by 0.63-0.7 eV. Common hosts such as mCBP, CBP, DPEPO have shallower HOMO energies which doesn't nest these fl-dopants. As a result, excimer formation is observed when doped in these different host materials. From DFT modelling, our data suggests that by decorating the aza-DiPYR compounds with electron donating functional groups in specific positions, we can destabilize the HOMO energy thus making it shallower. Following this approach, not only the HOMO/LUMO energies will be suitable to be hosted in host materials but we can also maintain the high quantum yield and the small singlet-triplet energy gap (ΔS₁/T₁≤0.3 eV) necessary used for developing blue fl dopant materials for hybrid WOLED.

TABLE 6
Singlet and Triplet energies deduced from calculations
and experimental data for aza-substituted DiPYRs

| | Calculated results^{(a)} | | | Experimental results^{(b)} | | |
|---|---|---|---|---|---|---|
| | S₁ | T₁ | AE(S₁/ | S₁ | T₁ | AE(S₁/ |
| | (eV/nm) | (eV/nm) | T₁) | (eV/nm) | (eV/nm) | T₁) |
| aD | 3.16 | 2.65 | 0.51 | 3.11 | 2.64 | 0.47 |
| | (392 nm) | (468 nm) | | (398 nm) | (469 nm) | |
| aDBPh₂ | 2.86 | 2.51 | 0.35 | 2.89 eV | 2.68 eV | 0.21 |
| | (433 nm) | (493 nm) | | (429 nm) | (463 nm) | |
| aDBT₂ | 2.65 eV | 2.37 eV | 0.28 | 2.82 eV | — | — |
| | (467 nm) | (523 nm) | | (440 nm) | | |
| α-aD | 2.78 | 2.39 | 0.39 | 2.86 | 2.56 | 0.30 |
| | (446 nm) | (518 nm) | | (434 nm) | (484 nm) | |
| γ-aD | 2.78 | 2.39 | 0.39 | 2.87 | 2.57 | 0.30 |
| | (446 nm) | (518 nm) | | (432 nm) | (482 nm) | |
| α-aID | 2.65 | 2.34 | 0.31 | 2.79 | 2.51 | 0.28 |
| | (467 nm) | (529 nm) | | (444 nm) | (494 nm) | |
| α-5OD | 2.79 | 2.47 | 0.32 | 2.77 | 2.51 | 0.26 |
| | (444 nm) | (502 nm) | | (447 nm) | (493 nm) | |
| αα-OD | 2.77 | 2.43 | 0.34 | 2.81 | 2.53 | 0.28 |
| | (447 nm) | (510 nm) | | (442 nm) | (490 nm) | |
| αγ-OD | 2.78 | 2.43 | 0.35 | 2.84 | 2.46 | 0.38 |
| | (446 nm) | (510 nm) | | (437 nm) | (503 nm) | |

^{(a)}TD-DFT B3LYP/6-311G**
^{(b)}Singlets are extrapolated from peak max of Fl (298 K) and Triplets from gated phos (77 K) in 2-MeTHF.

The photophysical characterizations are shown in FIG. 27. Each spectrum shows the absorption and emission profile for the respective aza-substituted DiPYRs in 2-methyltetrahydrofuran (MeTHF). All compounds have a small stoke shift gap between the absorption and emission. The singlet energies were extrapolated from the peak maximum of the fluorescence emission taken at room temperature, and the triplet energies were deduced from the gated phosphorescence emission taken at 77 K after 500 μs pulse-delay time from excitation. It's noteworthy to mention that the excited state lifetime of the singlet-state fluorescence is about 2 to 4 ns, and the triplet-state phosphorescence is long-lived (milliseconds). The quantum yield for these newly synthesized aza-substituted DiPYRs have maintained the high quantum yield (Φ_{f}≥0.85) like the parent α-aD with radiative decays of 10⁸ s⁻¹ and non-radiative decays of 10⁷ s⁻¹.

TABLE 7
Photophysical properties of blue fl dopant emitters in 2-MeTHF.

| | | τ | kᵣ | kₙᵣ |
|---|---|---|---|---|
| | Φ_{fl} | (ns) | (s⁻¹) | (s⁻¹) |
| aD | 0.42 | 2.11 | 2 × 10⁸ | 2.74 × 10⁸ |
| α-aD | 0.86 | 3.25 | 2.65 × 10⁸ | 4.31 × 10⁷ |
| γ-aD | 0.87 | 3.16 | 2.75 × 10⁸ | 4.11 × 10⁷ |
| α-aID | 0.87 | 3.82 | 2.27 × 10⁸ | 3.40 × 10⁷ |
| α-5OD | 0.84 | 3.48 | 2.41 × 10⁸ | 4.60 × 10⁷ |
| αα-OD | 0.84 | 3.28 | 2.56 × 10⁸ | 4.89 × 10⁷ |
| αγ-OD | 0.90 | 2.81 | 3.20 × 10⁸ | 3.56 × 10⁷ |
| aDBT₂ | 0.22 | 2.35 | 0.93 × 10⁸ | 3.32 × 10⁸ |
| aDBPh₂ | 0.30 | 2.08 | 1.44 × 10⁸ | 3.37 × 10⁸ |
| aCarD | 0.42 | 2.11 | 2.00 × 10⁸ | 2.09 × 10⁷ |
| α-aCarD | 0.76 | 6.74 | 1.13 × 10⁸ | 0.35 × 10⁷ |

Φ_{fl}: Quantum Yield τ: excited state lifetime kᵣ: radiative decay kₙᵣ: nonradiative decay

Table 6 summarizes the singlet and triplet energy gaps deduced from the calculated and experimental values. The newly synthesized aza-substituted DiPYRs have a small singlet-triplet energy gaps (ΔS₁/T₁≤0.4 eV) with a triplet energy above 2.50 eV making them suitable as blue dopant materials that can shuttle triplet excitons to green and red phosphors in WOLED. However, the αγ-OD compound has a triplet energy of 2.46 eV, making it difficult to transfer triplets to green phosphors (2.5 eV), but might be suitable for yellow and red emitters. The quantum yield for these compounds remained high (Φ_{f}≥0.85) in solution (MeTHF) as well as in a polymer matrix such as PMMA. All these derivatives are still to be tested in in blue OLEDs and hybrid WOLED. Our data suggests that the symmetric methoxy DiPYR (α-5OD) blue fl dopant (triplet energy above 2.51 eV) with shallower HOMO/LUMO energies (−5.92 eV/−2.38 eV) will be suitable to be hosted in the new host materials shown from the previous report.

Since the synthesis of the substituted α-aD core took 6-7 steps to make, it is worth considering other substitution sites which may effectively provide us a similar destabilization of the HOMO energy while increasing the steric bulk of the molecule to minimize any potential self-quenching when doped at high concentrations. Additional compounds were synthesized where —BF₂ was replaced with —B(Ph)₂ or —B(Tolyl)₂, named aDBPh₂ and aDBT₂ respectively. Their photophysical spectra are shared in FIG. 27. As shown in Table 6, the HOMO of aDBPh₂ obtained from electrochem is −5.62 eV, which is shallower than the −6.21 eV value of α-aD. The triplet of aDBPh₂ is also well above 2.50 eV, making them suitable as blue dopant materials that can shuttle triplet excitons to green and red phosphors in WOLED. However, the fast nonradiative rates and low quantum yields of aDBT₂ and aDBPh₂ compared to aD, suggests that the deactivation pathway observed in aD is enhanced by the addition of the bulky groups. A tactic we are using to circumvent this problem is by substituting bulky groups on the boron atom of the α-aD core. As we learned from the aD and α-aD core structures, the benzannulation should destabilize the T2 enough to minimize the contribution of intersystem, crossing as a nonradiative decay pathway. It is expected that, by adding phenyl groups or tolyl groups to the boron atom of α-aD, a comparable oxidation potential to that of aDBPh₂, but with quantum yield and lifetime values comparable to α-aD is achievable.

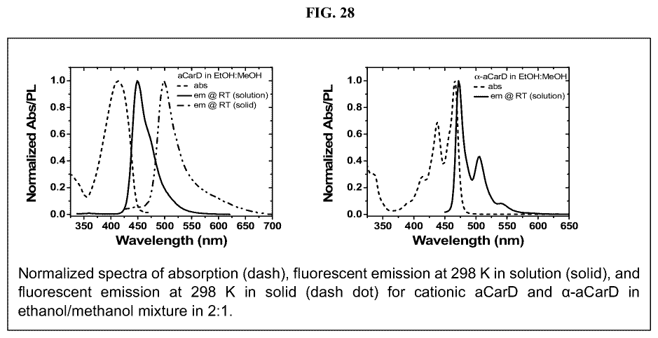

Additionally, cationic derivatives of the azaDIPYR core have been synthesized as shown in FIG. 28 which has emission wavelength red-shifted by 30-50 nm to its boron counterpart. Although the cationic derivatives may not be the best system for WOLED applications, making neutral derivatives by introducing groups such as carboxylic acid substituents in the structure. The deprotonation of the carboxylic acid proton produces an overall neutral structure which will have a reasonable oxidation and reduction potentials as fluorescent blue dopants.

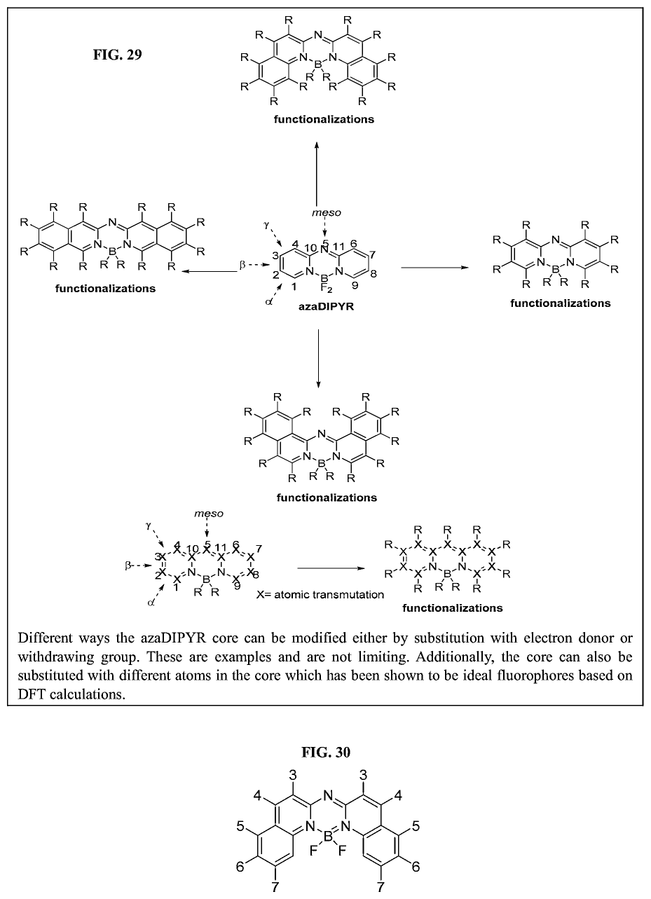

Techniques to modify the azaDIPYR, α-azaDIPYR, and g-azaDIPYR core, which are the foundations upon which the modeling and photophysical descriptions are built, have been disclosed herein. Subsequently, a series of modifications to the basic structures are disclosed. In all cases, any —CH— position may be functionalized as —CR—, where —R is an alkyl, aryl, donor, or acceptor group, is a deuterated or otherwise isotopically functionalized atom, or is another atom, either a metal or main group element. This also includes benzannulation and applies to any —CH— position in any of the derivatives outlined herein. Any X shown in FIG. 29 represents any atomic transmutation in the core structure that may be of interest including but not limited to multiple nitrogen atoms in the core structure.

### Substituted azaDIPYR/DIPYR Dves

The azaDIPYR core based on the device data disclosed herein are interesting material which has photophysical properties to BODIPY but are synthetically more accessible to modify. The azaDIPYR family are great candidates to use as blue fluorescent dopants for WOLED. As shown from our data from the molecules illustrated in FIG. 27 that tuning the HOMO/LUMO energies can be achieved by addition of substituents or asymmetry to the core structure. Development of blue fluorophores from the core azaDIPYR and α-azaDIPYR can be expanded with other electron donating, electron donating and other substituents in addition to the methoxy, isopropyl, tolyl and phenyl groups. The Boron atom in the core structure can also be replaced with other metals.

Additionally, by tactful placement of electron-withdrawing/electron-donating groups, design parameters can be tuned as indicated by the DFT results summarized in Table 8.

TABLE 8
DFT (B3LYP/6-31G**) calculated properties for α-aD derivatives.
(The candidates are named based on the nature and site
of substitution following the scheme in FIG. 30)

| | | | | | ΔS₁/ | | |
|---|---|---|---|---|---|---|---|
| | S₁ | S₁ | T₁ | T₁ | T₁ | HOMO | LUMO |
| | (eV) | (nm) | (eV) | (nm) | (eV) | (eV) | (eV) |
| α-aD | 2.73 | 454 | 2.39 | 519 | 0.34 | −5.62 | −2.07 |

OMe

| | | | | | | | |
|---|---|---|---|---|---|---|---|
| α-aD-(OMe)₂-3 | 2.64 | 470 | 2.25 | 550 | 0.38 | −5.31 | −1.90 |
| α-aD-(OMe)₂-4 | 2.92 | 425 | 2.67 | 465 | 0.25 | −5.41 | −1.62 |
| α-aD-(OMe)₂-5 | 2.79 | 444 | 2.47 | 502 | 0.33 | −5.45 | −1.81 |
| α-aD-(OMe)₂-6 | 2.49 | 498 | 2.19 | 567 | 0.31 | −5.25 | −1.95 |
| α-aD-(OMe)₂-7 | 2.65 | 468 | 2.36 | 525 | 0.29 | −5.36 | −1.90 |

Mes

| | | | | | | | |
|---|---|---|---|---|---|---|---|
| α-aD-(Mes)₂-3 | 2.70 | 459 | 2.38 | 521 | 0.32 | −5.58 | −2.04 |
| α-aD-(Mes)₂-4 | 2.71 | 457 | 2.41 | 515 | 0.30 | −5.52 | −1.96 |
| α-aD-(Mes)₂-5 | 2.71 | 458 | 2.40 | 517 | 0.31 | −5.55 | −2.01 |
| α-aD-(Mes)₂-6 | 2.66 | 466 | 2.36 | 525 | 0.30 | −5.58 | −2.07 |
| α-aD-(Mes)₂-7 | 2.63 | 472 | 2.38 | 522 | 0.25 | −5.55 | −2.07 |

CN

| | | | | | | | |
|---|---|---|---|---|---|---|---|
| α-aD-(CN)₂-3 | 2.64 | 470 | 2.37 | 523 | 0.27 | −6.28 | −2.79 |
| α-aD-(CN)₂-4 | 2.38 | 521 | 2.04 | 608 | 0.34 | −6.30 | −3.14 |
| α-aD-(CN)₂-5 | 2.62 | 473 | 2.31 | 536 | 0.31 | −6.31 | −2.87 |
| α-aD-(CN)₂-6 | 2.66 | 466 | 2.36 | 525 | 0.30 | −6.35 | −2.89 |
| α-aD-(CN)₂-7 | 2.62 | 474 | 2.34 | 531 | 0.28 | −6.30 | −2.83 |

F

| | | | | | | | |
|---|---|---|---|---|---|---|---|
| α-aD-(F)₂-3 | 2.68 | 462 | 2.30 | 540 | 0.39 | −5.80 | −2.34 |
| α-aD-(F)₂-4 | 2.85 | 434 | 2.56 | 485 | 0.30 | −5.84 | −2.14 |
| α-aD-(F)₂-5 | 2.78 | 446 | 2.43 | 510 | 0.35 | −5.86 | −2.26 |
| α-aD-(F)₂-6 | 2.64 | 470 | 2.30 | 539 | 0.34 | −5.73 | −2.28 |
| α-aD-(F)₂-7 | 2.74 | 453 | 2.40 | 516 | 0.34 | −5.78 | −2.25 |

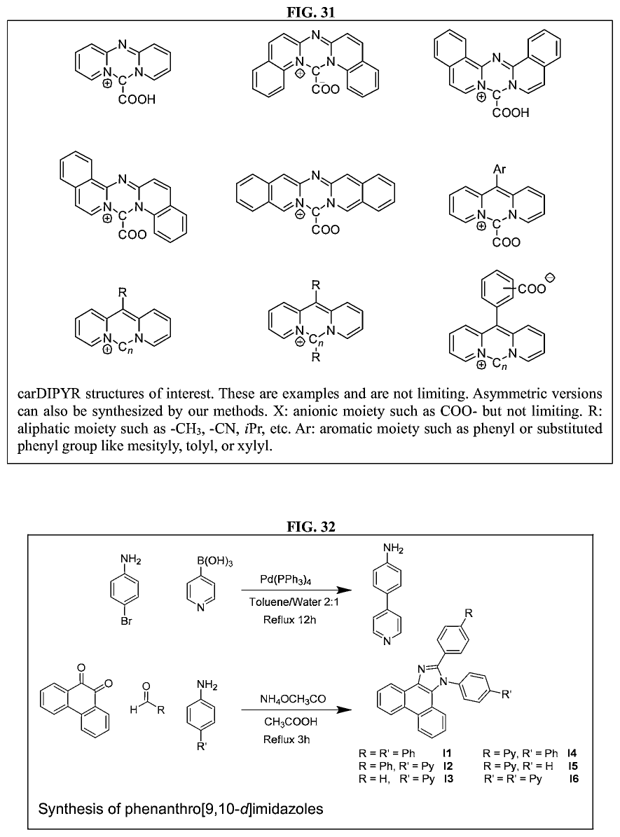

Carbon-coordinated azaDIPYRs (acarDIPYRs): Boron has been shown to be an excellent coordinating atom for DIPYRs and BODIPYs, forming stable pseudo-aromatic fluorophores with high photoluminescent quantum yields. We have also shown that carbon can be used in lieu of a metal or main group element to produce bright emission in the solution and solid states. The resulting unsubstituted dye carDIPYR is cationic, with a delocalized positive charge shared between both nitrogens. The counteranion can be exchanged with for suitable anion (for example halogens, such as iodide and bromide, have been used, as has PF6—) to meet specific application requirements. We have shown in FIG. 27, that we had successfully characterized a cationic derivative of azaDIPYR (aCarD) and α-azaCARDIPYR (α-aCarD). We are interested in these cationic species, but more specifically the neutral derivatives of these systems which are shown in FIG. 31. Similar substitution patterns can be applied to the ones mentioned in the substituted azaDIPYR/DIPYR dyes section.

### Host Materials for fl/ph Hybrid WOLEDs

Using the data and techniques disclosed herein, it is also possible to identify and prepare host materials that can nest the energy levels of blue fluorescent dopant and host the singlet and triplet energy of green, yellow and red phosphorescent emitters. Blue fluorescent emitters with strong blue fluorescence in the solid-state that can act as host material for green, yellow and red phosphors are also presented in this section.

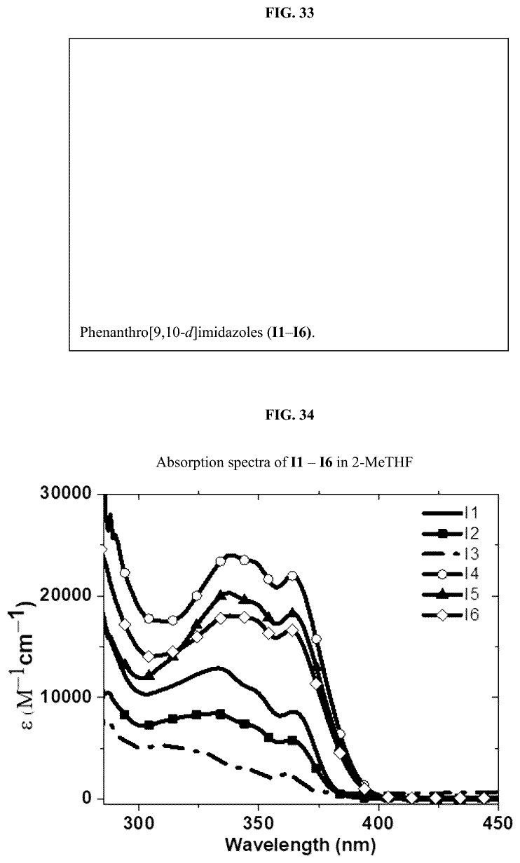

We have identified phenanthro[9,10-d]imidazoles as potential materials that can serve these two purposes. Based on our modeling studies, we selected and synthesized six phenanthro[9,10-dimidazoles, i.e. I1-I6 (using Schemes 3a, 3b shown in FIGS. 32 and 33, respectively), also identified as “H2P” compounds previously, which have the potential for use as hosts, dopants and neat fluorescent emitters for hybrid-fluorescent white organic light emitting diode (WOLED). The materials are obtained in high yields (>80%) and are purified by filtration followed by sublimation.

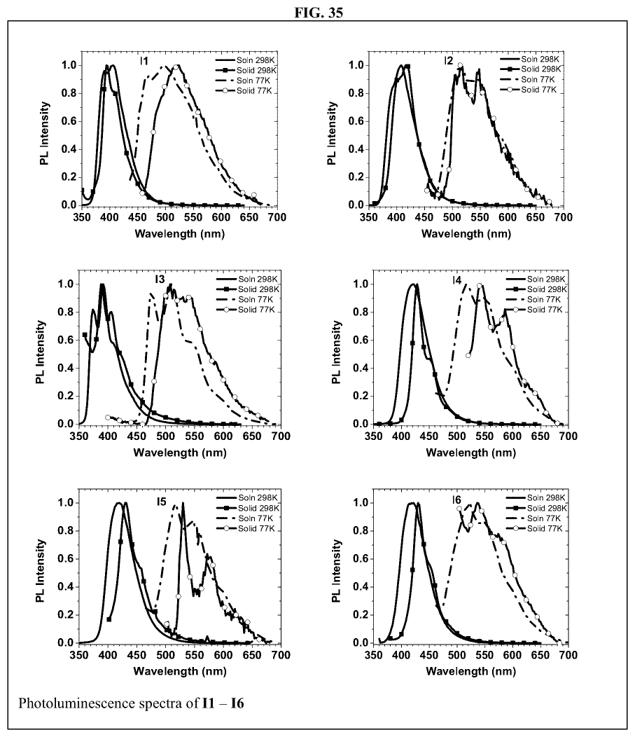

All the materials absorb in the range 310-380 nm with moderate extinction coefficients (>10⁴ M⁻¹ cm⁻¹) (FIG. 34). The emission spectra in 2-MeTHF of I1-I3 blue-shifted relative to the emission spectra of I4-I6 (FIGS. 35). I1, I4, I5, and I6 have near unity photoluminescence quantum yield (PLQY), whereas I2 and I3 have PLQY less than 20% in 2-MeTHF (Table 9). To check the potential use of these materials as neat fluorescent emitters, we studied photophysical properties of these materials in neat films and solid powder. I1, I4, I5, and I6 retained high PLQY as neat films (Table 9), with their emission spectra red-shifted in neat film and solid relative to their energies in solution. Furthermore, the fluorescent spectrum of I4, I5, and I6 broadened and have the same onset with a widely used neat fluorescent emitter for hybrid WOLED (4P-NPD).

TABLE 9
Photophysical Properties of 1-16

| | S₁ | S₁ | T₁ | T₁ | PLQY | PLQY |
|---|---|---|---|---|---|---|
| | (Soln) | (Solid) | (Soln) | (Solid) | (%)^{a} | (%)^{b} |
| I1 | 373 | 377 | 441 | 468 | 99 | 95 |
| I2 | 375 | 382 | 477 | 490 | 28 | — |
| I3 | 365 | 370 | 460 | 478 | 28 | — |
| I4 | 390 | 412 | 478 | 520 | 99 | 55 |
| I5 | 390 | 406 | 480 | 520 | 99 | 81 |
| I6 | 386 | 410 | 475 | 495 | 99 | 83 |
| 4P-NPD | 380 | 415 | — | 525 | 69 | 57 |

^{a}Measured in 2-MeTHF.
^{b}neat film.
All S₁ and T₁ values are in nm

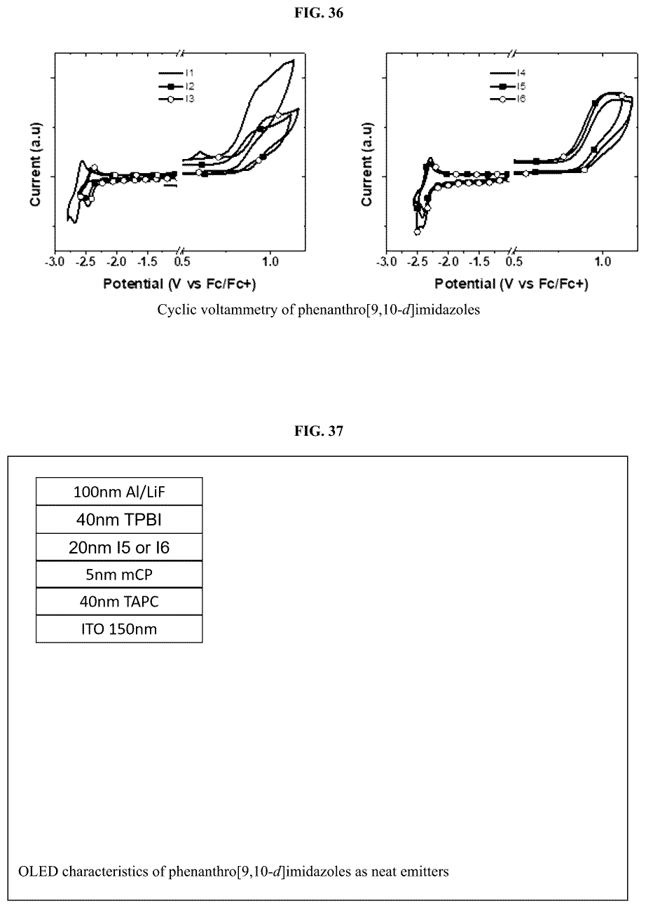

The electrochemical properties of these materials are summarized in Table 10. All the materials show irreversible oxidation and reversible reduction (scan rates of 0.1 V/s and 10 V/s). (FIG. 36). Their HOMO and LUMO levels are calculated from their oxidation and reduction potentials, respectively and are similar with small variations.

Because of their appropriate singlet and triplet energies, these materials can serve as host for α-aD. We doped 1 wt % of α-aD into the phenanthro[9,10-d]imidazole hosts and high PLQYs were obtained (>80%).

Prior to fabricating WOLED with these materials as hosts or neat emitter materials, we doped a green (Ir(ppy)₃), a yellow (Ir(bt)₂acac) and a red (PQIr) phosphor into these host materials (Table 10). The PLQY of the yellow and red phosphors are maintained high (>70%), whereas the efficiency of the green phosphor is low in these host materials. Preliminary results using I2, I4, I5 and I6 as fluorescent neat emitters and as host materials in WOLED are discussed below.

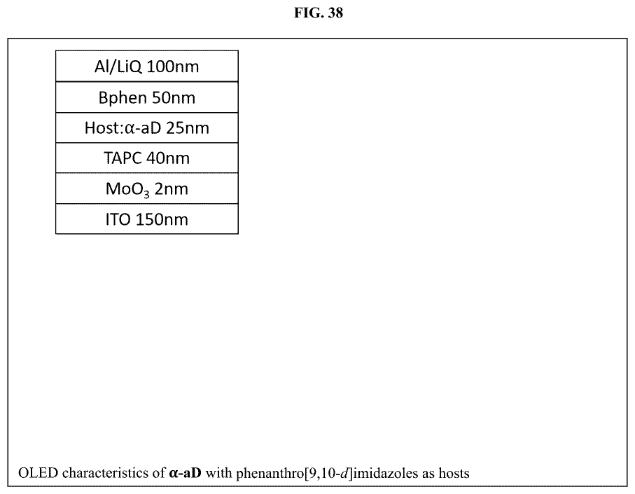

The host materials were utilized to fabricate blue fluorescence monochromatic devices and hybrid-WOLEDs. The first set of monochromatic devices shown in FIG. 37 used either I5 or I6 as neat fluorescence emitter in the emissive layer. The second set, used I2, I4 and I5 as host materials for α-aD (FIG. 38). The EL spectrum of I5 and I6 in the first set of devices agree with their PL spectrum in neat films. Their transport properties and efficiencies are similar, reaching high EQE of 3.5 which is close to the theoretical maximum efficiency of fluorescent emitters (FIG. 37). Similarly, the α-aD based devices achieved high EQE (peak=3.5%) close to the theoretical maximum (FIG. 38). The EL spectrum of α-aD in I2, I4 or I5 host matches its PL spectrum, indicating exciton confinement on the dopant.

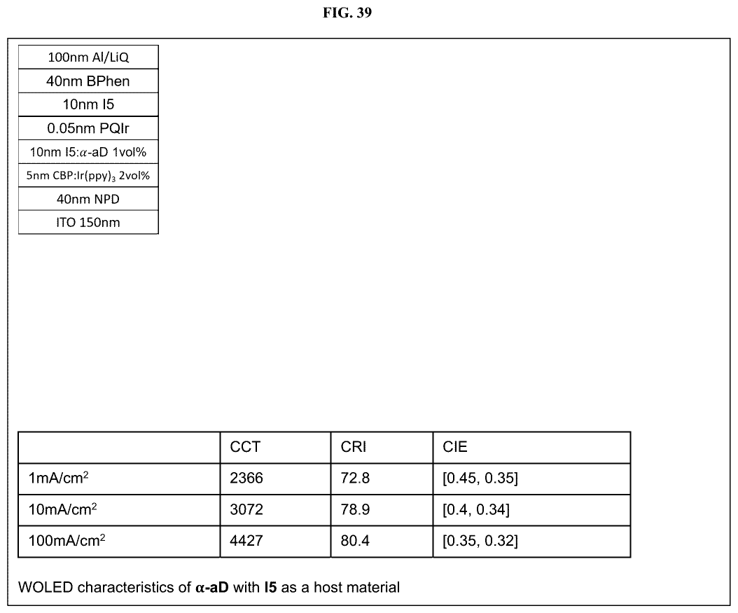

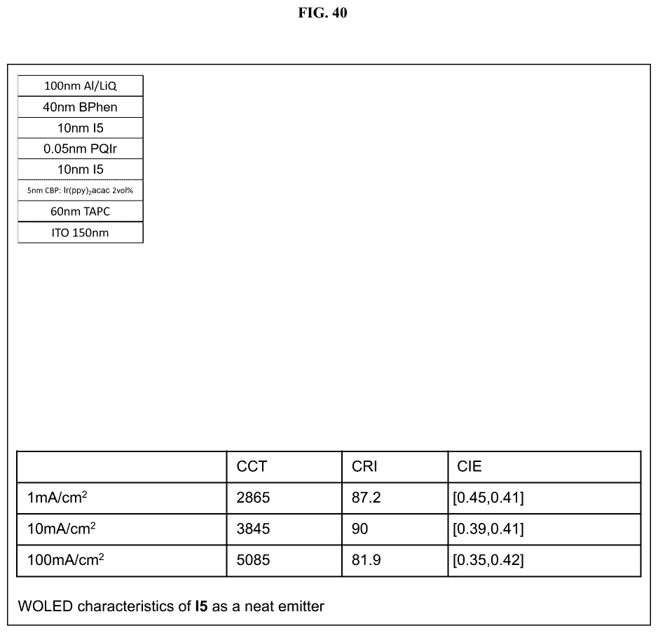

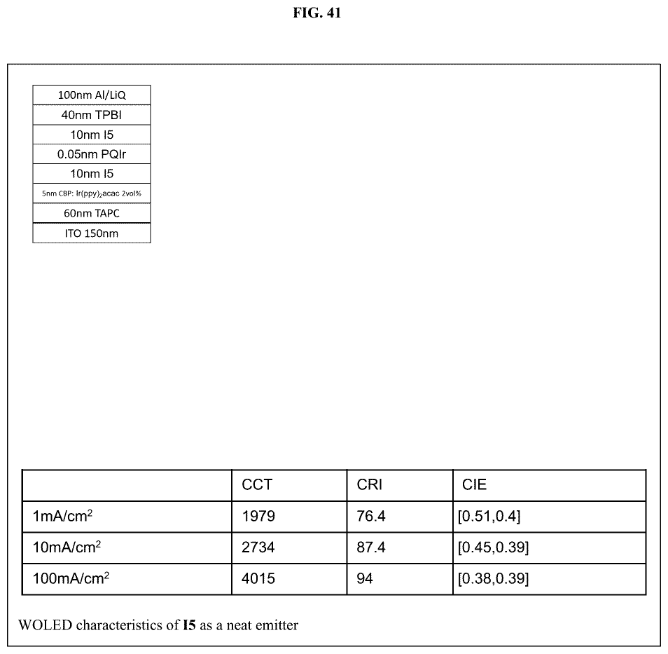

I5 was chosen to fabricate hybrid WOLED since good efficiencies were obtained with I5 as neat fluorescent emitter and as a host in monochromatic devices. We tested different devices with and without -aD to examine the possibility of using I5 as both a neat emitter and a host. The device structure with the best performance is detailed in FIGS. 39, 40, and 41. Device employing I5 as a host for -aD has moderate EQE and luminous efficacy at low current density (FIG. 39). Slightly higher values for EQE and luminous efficacy are obtained with I5 as a neat fluorescent emitter (FIG. 40).

TABLE 10
Electrochemical properties of phenanthro[9,10-d]imidazoles

| | Eₒₓ | E_{red} | | |
|---|---|---|---|---|
| | (V)^{a} | (V)^{a} | HOMO^{b} | LUMO^{b} |
| I1 | +0.86 | −2.61 | −5.81 | −1.70 |
| I2 | +0.87 | −2.44 | −5.82 | −1.89 |
| I3 | +0.90 | −2.44 | −5.86 | −1.89 |
| I4 | +0.91 | −2.32 | −5.87 | −2.03 |
| I5 | +0.91 | −2.35 | −5.87 | −2.00 |
| I6 | +0.96 | −2.36 | −5.94 | −2.00 |

^{a}reference to ferrocene ^{b}experimental values in eV

TABLE 11
PLQY data of spin coated films of Ir(ppy)₃, Ir(bt)₂acac,
and PQIr in the phenanthro[9,10-d]imidazole hosts

| PLQY | I1 | I2 | I3 | I4 | I5 | I6 |
|---|---|---|---|---|---|---|
| 10 wt % Ir(ppy)₃ | 65 | 49 | 56 | 39 | 30 | 36 |
| 10 wt % Ir(bt)₂acac | 80 | 66 | 84 | 83 | 80 | 78 |
| 10 wt % PQIr | 70 | 69 | 67 | 64 | 69 | 66 |
| Neat Host | 80 | — | — | 55 | 81 | 83 |

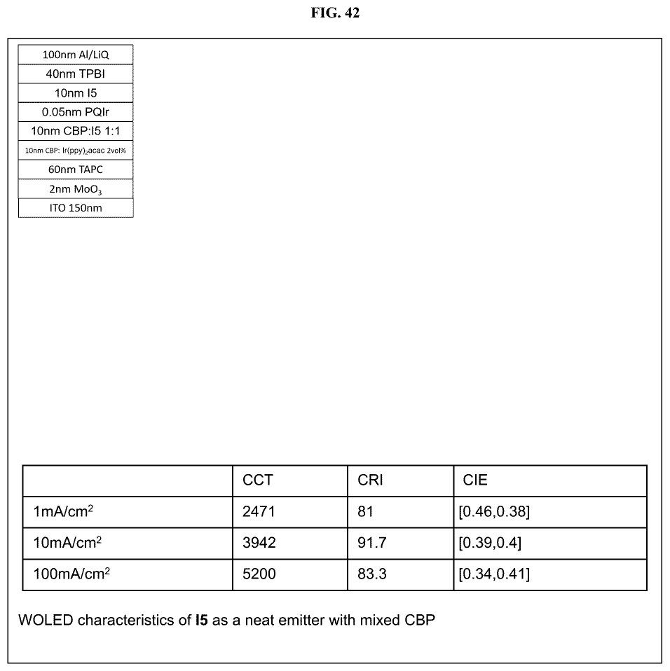

Compared to the device in FIG. 39, the device in FIG. 40 has well balanced spectrum with CIE coordinates of (0.39,0.41) and color rendering index of 90 at 10 mA/cm². Transport layer thicknesses have been adjusted for optimized outcoupling in FIG. 40. Higher luminous efficacy and EQE at low current density are obtained with TPBi as an electron transport layer (FIG. 41). When CBP is mixed with I5 in a 1:1 ratio, EQE and power efficiency is enhanced to 22.5% and 57.8 lm/W, respectively as can be seen in FIG. 42. All devices currently suffer from drastic efficiency roll-off at high currents. The efficiency roll-off must be improved for this structure to have any practical value. The luminous power efficacy drops to 17.3 lm/W at a forward viewing luminance of 500 nits and further decreases to 12.2 lm/W at 1000 nits. Currently, the device in FIG. 42 has a well-balanced white spectrum at a current density of 10 mA/cm2 with CIE coordinates of (0.39,0.4) and color rendering index of 91.7.

Based on these data, a hybrid emissive layer may be constructed following the general parameters that have been found for suitable host and dopant combinations. For example, the data in Tables 9-11 above suggest that I1, I4, I5, and I6 may be suitable fluorescent dopants for use with combinations of hosts and phosphorescent dopants as disclosed herein. These combinations of materials may provide the desirable energy state transitions and performances disclosed herein and referenced previously for early hybrid layers, but without the drawbacks of prior work as disclosed herein.

Generalizing the results of the studies disclosed herein, it has been determined that a hybrid emissive layer may be structured and fabricated such that it includes three materials 1, 2, 3, having the following energy level relationships for the first singlet and triplet states S1, T1, respectively:

$$T {1}_{2} ⩾ T {1}_{1} ; S {1}_{2} ⩽ S {1}_{1} ; and T {1}_{3} < T {1}_{1} .$$

In an embodiment, these materials may correspond to the host material, the fluorescent dopant, and the phosphorescent dopant, respectively. That is, in a hybrid emissive layer comprising a host material H, fluorescent dopant F, and phosphorescent dopant P, the singlet and triplet state energy levels may be:

$$T {1}_{F} ⩾ T {1}_{h} ; S {1}_{F} ⩽ S {1}_{H} ; and T {1}_{P} < T {1}_{H} .$$

Where T1_{H} is the first triplet energy level of the host, T1_{F} is the first triplet energy level of the fluorescent dopant, S1_{F} is the first singlet energy level of the fluorescent dopant, and T1_{P} is the first triplet energy level of the phosphorescent dopant. Examples of suitable energy levels may be seen, for example, by comparing FIG. 9B and FIG. 35, which shows energy levels for I5 as disclosed herein. The single and triplet energy levels of the fluorescent dopant, for example as shown by the dashed line and solid line 920, respectively, in FIG. 9B are nested between the singlet and triplet energy levels of the host, in this example I5, given by the solution and solid lines in FIG. 35. As shown by the data disclosed herein, this arrangement can provide efficient WOLED arrangements with enhanced lifetime and operation relative to earlier hybrid emissive layer configurations, which did not require or exhibit the same energy level arrangements. Arrangements as disclosed herein do not lead to, and may prevent, triplet energy trapping the fluorescent dopant. In contrast, prior devices and materials do not provide for this, because the triplet energy level for the fluorescent dopant typically was well below the triplet level of the host. As previously disclosed, compounds I1, I4, I5, and I6 may be suitable for use in a hybrid layer as disclosed herein, though the embodiments disclosed herein are not limited to these particular materials.

In some embodiments, an OLED incorporating a hybrid emissive layer as disclosed herein may the host material may emit only from the first material, i.e., the fluorescent dopant in some embodiments. Furthermore, in some embodiments the first material, which may be a host material, may be, or may include the fluorescent dopant material. That is, the same material may serve as both the host of the emissive layer and as the fluorescent dopant as disclosed herein. In some cases, the OLED may include additional emissive layers, which may include the same material as the host/fluorescent emissive dopant. Such OLEDs still may exhibit all emission from the first material.

In some embodiments, it may be preferable to maintain a minimum separate between energy levels of the materials. For example, it may be preferred for T1_{H} is at least 0.1 eV greater than T1_{P} and/or for T1_{F} to be at least 0.1 eV greater than T1_{H}. Such arrangements are shown and suggested, for example, by Tables 2.1b and 9-11 herein. Alternatively or in addition, it may be desirable for the S1-T1 energy gaps to fall within specific ranges, as disclosed herein. In particular, it may be desirable for the fluorescent dopant and/or the host material to have relatively small S-T energy gaps as previously described. As a specific example, it may be preferred for the S1_{F}-T1_{F} gap to be 0.05 to 0.8 eV, 0.1 to 0.8 eV, or less than 0.4 eV. In some embodiments, it also may be preferred for individual energy levels to fall within specific ranges or minimums. For example, as shown by Tables 2.1b and 9-11, it may be preferred for S1_{H} to be at least 3.5 eV, T1_{H} to be in the range 2.4-2.6 eV, and/or T1_{P} to be in the range 1.7-2.5 eV.

The use of materials and combinations of materials as disclosed herein may allow for more efficient and consistent devices than may be achieved using prior hybrid emissive layers or conventional device configurations, as shown by the data presented herein such as at FIGS. 38-42 and Tables 9-11.

### Experimental

Synthesis techniques for the dopant and host materials disclosed herein are described below. General synthetic scheme for the preparation of phenanthro[9,10-d]imidazoles.

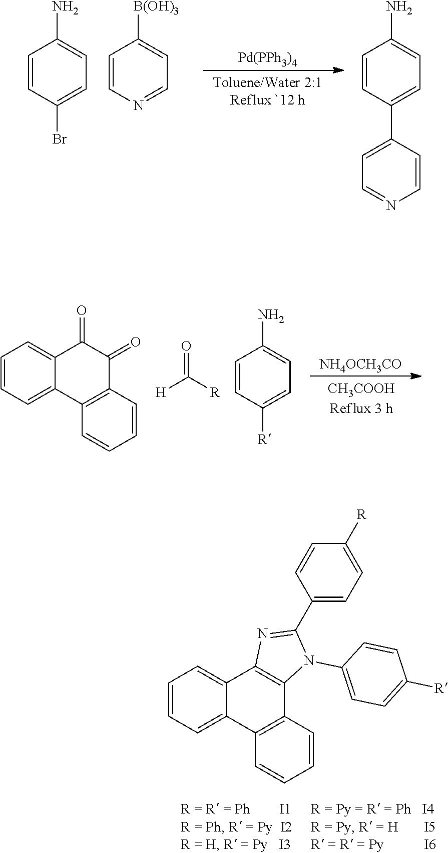

4-(pyridin-4-yl)aniline: In a 250 ml one-necked round bottomed flask, 4-pyridineboronic acid (14.3 g, 116.2 mmol), 4-bromoaniline (10.0 g, 58.1 mmol), tetrakis(triphenylphosphine)palladium(0) (185 mg, 0.174 mmol), and Na₂CO₃ (43.1 g, 406.9 mmol) were added. The flask is purge and backfilled with nitrogen three times. 180 ml of degassed DMF/H₂O (9:2) was canula transferred to the flask and heated to reflux overnight. The reaction mixture was then cooled to room temperature and diluted with DCM. The organic layer was separated, and the solvent removed under reduced pressure. The product was then precipitated from DCM to yield the pale yellow solid (5.30 g, 53%).

1,2-di([1,1′-biphenyl]-4-yl)-1H-phenanthro[9,10-d ]imidazole (II) : A mixture of phenanthrene-9,10-dione (2.00 g, 9.61 mmol), [1,1′-biphenyl]-4-amine (1.95 g 11.53 mmol), [1,1′-biphenyl]-4-carbaldehyde (1.75 g, 9.61 mmol) and ammonium acetate (1.48 g, 19.21 mmol) in glacial acetic acid (30 mL) was refluxed for 3 h. The precipitate was filtered and washed with aqueous NaOH deionized water. The residue is dried and sublimed at 250° C. and 1.2×10⁻⁶ torr to give pure product. White solid (4.20 g, 8.04 mmol, 84%).

2-([1,1′-biphenyl]-4-yl)-1-(4-(pyridin-4-yl)phenyl)-1H-phenanthro[9,10-d]imidazole (I2) : A mixture of phenanthrene-9,10-dione (2.20 g, 10.57 mmol), 4-(pyridin-4-yl)aniline (2.16 g 12.68 mmol), [1,1′-biphenyl]-4-carbaldehyde (1.93 g, 10.57 mmol) and ammonium acetate (1.63 g, 21.13 mmol) in glacial acetic acid (70 mL) was refluxed for 3 h. The precipitate was filtered and washed with aqueous NaOH deionized water. The residue is dried and sublimed at 280° C. and 1.2×10⁻⁶ torr to give pure product. White solid (2.13 g, 4.06 mmol, 39%).

2-phenyl-1-(4-(pyridin-4-yl)phenyl)-1H-phenanthro[9,10-d]imidazole (I3): A mixture of phenanthrene-9,10-dione (1.50 g, 7.20 mmol), 4-(pyridin-4-yl)aniline (1.47 g 8.64 mmol), benzaldehyde (0.77 g, 7.20 mmol) and ammonium acetate (1.11 g, 14.41 mmol) in glacial acetic acid (50 mL) was refluxed for 3 h. The precipitate was filtered and washed with aqueous NaOH deionized water. The residue is dried and sublimed at 270° C. and 1.2×10⁻⁶ torr to give pure product. White solid (1.18 g, 2.64 mmol, 37%).

1-([1,1′-biphenyl]-4-yl)-2-(4-(pyridin-4-yl)phenyl)-1H-phenanthro[9, 10-d]imidazole (I4): A mixture of phenanthrene-9,10-dione (2.00 g, 9.61 mmol), [1,1′-biphenyl]-4-amine (1.95 g 11.53 mmol), 4-(pyridin-4-yl)benzaldehyde (1.76 g. 9.61 mmol) and ammonium acetate (1.48 g. 19.21 mmol) in glacial acetic acid (70 mL) was refluxed for 3 h. The precipitate was filtered and washed with aqueous NaOH deionized water. The residue is dried and sublimed at 270° C. and 1.2×10⁻⁶ torr to give pure product. White solid (4.20 g, 8.02 mmol, 84%).

1-phenyl-2-(4-(pyridin-4-yl)phenyl)-1H-phenanthro[9,10-d]imidazole (I5): A mixture of phenanthrene-9,10-dione (2.00 g, 9.61 mmol), aniline (1.07 g 11.53 mmol), 4-(pyridin-4-yl)benzaldehyde (1.76 g, 9.61 mmol) and ammonium acetate (1.48 g, 19.21 mmol) in glacial acetic acid (70 mL) was refluxed for 3 h. The precipitate was filtered and washed with aqueous NaOH deionized water. The residue is dried and sublimed at 270° C. and 1.2×10⁻⁶ torr to give pure product. White solid (3.65 g, 8.15 mmol, 85%).

1,2-bis(4-(pyridin-4-yl)phenyl)-1H-phenanthro[9,10-d]imidazole (I6): A mixture of phenanthrene-9,10-dione (2.50 g, 12.01 mmol), 4-(pyridin-4-yl)aniline (2.45 g 14.41 mmol), 4-(pyridin-4-yl)benzaldehyde (2.20 g, 12.01 mmol) and ammonium acetate (1.85 g, 24.01 mmol) in glacial acetic acid (80 mL) was refluxed for 3 h. The precipitate was filtered and washed with aqueous NaOH deionized water. The residue is dried and sublimed at 270° C. and 1.2×10⁻⁶ torr to give pure product. White solid (4.30 g, 8.20 mmol, 68%).

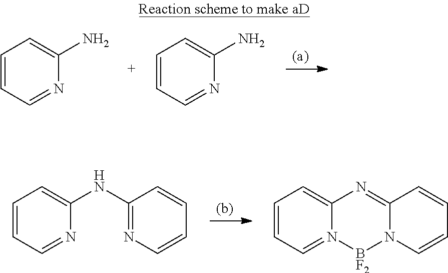

Synthesis of aD: (a) 2,2′-dipyridylamine (aD ligand): A reported procedure was followed (cite); bis(2-diphenylphosphinophenyl) ether(498.99 mg, 926.51 μmol), 2-bromopyridine(3.66, 23.16 mmol), 2-aminopyridine (2.18 g, 23.16 mmol) and t-BuONa (3.12 g, 32.43 mmol) were purged with nitrogen gas in a re-sealable shlenk flask where a degassed dry toluene is cannula transferred. Pd(OAc)₂ catalyst was added to the air free flask and refluxed in a 110° C. oil bath for 24 hours. The reaction mixture is cooled to room temperature and diluted with THF and ethyl ether. The solid precipitate was filtered, concentrated, and purified via silica gel column chromatography (2% MeOH/CH₂Cl₂). An alternative route is to purchase the commercially available 2,2′-dipyridylamine.

(b) azaDIPYR (aD): All reagents were purchased from Sigma Aldrich and used without purification. Anhydrous 1,2 dichloroethane was purchased from EMD Millipore. A solution of the 2,2′-dipyridylamine ligand (300 mg, 1.75mmol) in dry 1,2-dichloroethane was prepared in an N2-purged schlenk flask equipped with a magnetic stir bar and fitted with a reflux condenser. The flask was submerged in a preheated oil bath and brought to reflux, at which time 2.0 eq, boron trifluoride diethyl etherate (497.40 mg, 3.50 mmol) were added dropwise. The solution was stirred for 2 hours at reflux, then cooled to room temperature and treated with 5 eq. N,N diisopropylethylamine (1.53 mL, 8.70 mmol). The solution was washed with water and the aqueous layer was separated and extracted three times with DCM. The total organic extractions were filtered, and reduced concentrated by rotary evaporation. The products were purified by silica gel flash chromatography with the eluent 50% dichloromethane in hexanes.

(a) 2,2′-diquinolylamine (α-aD ligand): A reported procedure was followed (cite); bis(2-diphenylphosphinophenyl) ether(395.03 mg, 733.48 μmol), 2-bromoquinoline(3.00 g, 18.34 mmol), 2-aminoquinoline (2.78 g, 19.25 mmol) and t-BuONa (2.47 g, 25.67 mmol) were purged with nitrogen gas in a re-sealable shlenk flask where degassed dry toluene is cannula transferred. Pd(OAc)₂ (164.67, 733.48 μmol) catalyst was added to the air free flask and refluxed in a 110° C. oil bath for 24 hours. The reaction mixture is cooled to room temperature and diluted with THF and ethyl ether. The solid precipitate was filtered, concentrated, and purified via silica gel column chromatography (2% MeOH/CH₂Cl₂). A white solid is isolated upon purification (40-80% yield).

(b) α-azaDIPYR (α-aD): All reagents were purchased from Sigma Aldrich and used without purification. Anhydrous 1,2 dichloroethane was purchased from EMD Millipore. A 15 mM solution of 2,2′-diquinolylamine in dry 1,2-dichloroethane was prepared in an N₂-purged schlenk flask equipped with a magnetic stir bar and fitted with a reflux condenser. The flask was submerged in a preheated oil bath and brought to reflux, at which time 2.0 eq, boron trifluoride diethyl etherate were added dropwise. The solution was stirred for 2 hours at reflux, then cooled to room temperature and treated with 5 eq. N,N diisopropylethylamine, causing the precipitate to dissolve. The solution was washed with water and the aqueous layer was separated and extracted three times with dichloromethane. The organic layers were combined, dried over sodium sulfate, filtered, and reduced concentrated by rotary evaporation. The products were purified by silica gel flash chromatography with the eluent 80% 2% MeOH/CH₂Cl₂ solvent mixture in hexanes. For further purification, the material was sublimed. 1H).

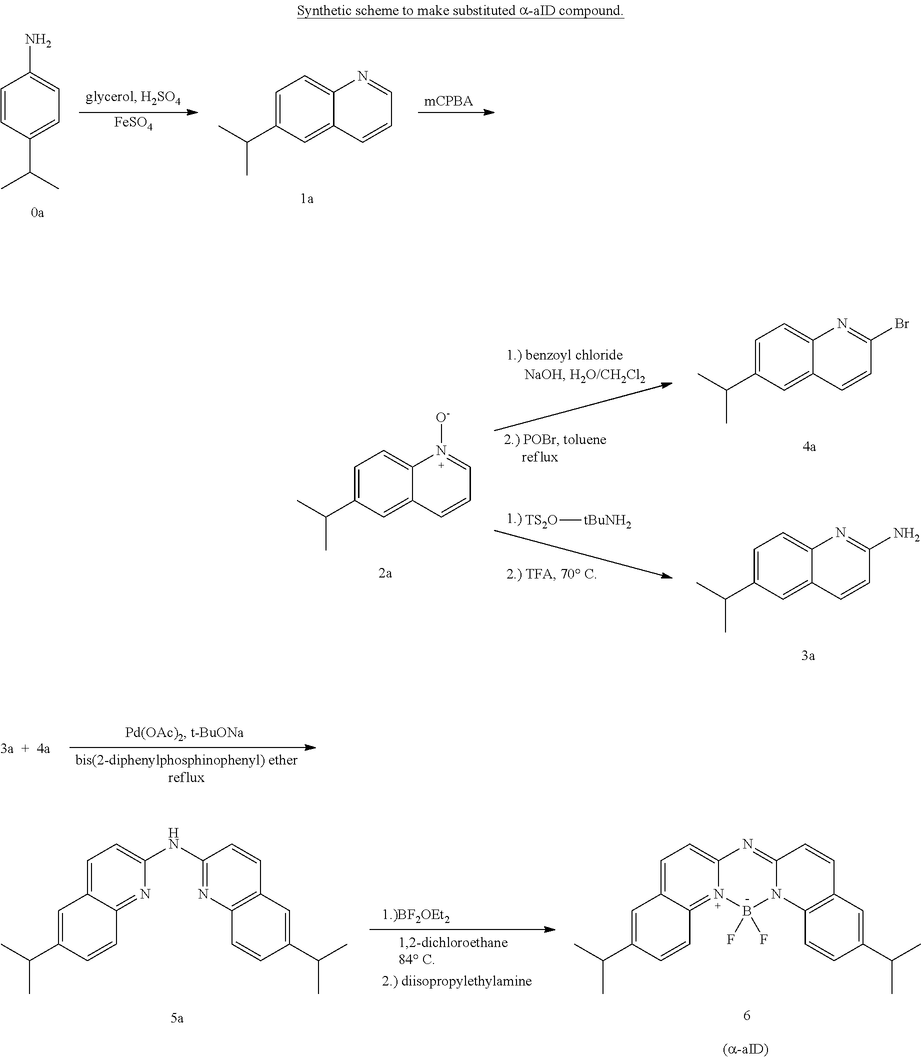

6-isopropylquinoline (1a). 4-isopropylaniline (20 g, 0.147 mole), nitrobenzene (9.86 ml, 0.096 mole), Glycerol (55.85 g, 0.606 mole), and FeSO₄.7H₂O (5.14 g, 18.49 mole) were added to a three-neck round bottom flask. While the flask was kept in an ice bath, H₂SO₄ (25 ml, 0.473 mole) was added slowly to the reaction mixture. After the addition was completed, the ice bath was removed followed by refluxing the mixture for 20 h under inert conditions. After cooling to room temperature, the pH of the solution was adjusted to pH 7 with 50% NaOH aq. Then, solution was extracted with diethyl ether. After the extraction, MgSO₄ was used as a drying agent. Filtration followed by evaporation to give a brown liquid. The product was isolated by reduced pressure distillation to yield the desired light-yellow liquid (yield 20%).

6-isopropylquinoline-1-oxide (2a). Compound (1a) (10.2 g, 0.059 mole) was dissolved in one-neck round bottom flask with CH₂Cl₂ (50 ml). M-chloroperoxybenzoic acid (m-CPBA) (12.33 g, 0.071 mole) was added slowly the stirred solution at room temperature. The reaction was stirred overnight. Next, saturated NaHCO₃ aq solution was added to stirring solution until no CO₂ gas bubbles were observed anymore. Then, pH was adjusted to 10 with NaOH aq solution and extracted with CH₂Cl₂ 50 ml three times. The solution was dried over MgSO₄. The solvent was removed under reduced pressure. The crude product was then purified by silica gel column chromatography (2% Methanol/CH₂Cl₂). White pale-yellow solid was afforded at 60% yield.

6-isopropylquinoline-2-amine (3a). To a round bottom flask, compound (2a) (2.55 g, 0.013 mole) and 30 ml of trifluorotoluene (7.16 ml, 0.068 mole) were mixed in 20 ml of chloroform. After compound (2a) was dissolved, the mixture was cooled to 0° C. with an ice bath. T-butylamine (7.16 ml, 0.068 mole) was added slowly followed by Ts₂O (8.89 g, 0.027 mole). The reaction was left to stir for two hours. If the reaction were not completed, portions of t-butylamine (0.6 equiv, to 4.0 equiv.) and Ts₂O (0.3 equiv, to 2.3 equiv.) would be added until the reaction is completed. The reaction was then treated with 25 ml TFA at 70° C. for overnight under inert atmosphere. After that, most of the solvents were removed under reduced pressure and them the concentrated oil residue was diluted with CH₂Cl₂ and quenched with 50% of aq solution of NaOH to pH 10. The solution was extracted with CH₂Cl₂ three times, dried over MgSO₄, and removed under reduced pressure. The crude product was then purified using a silica gel column chromatography (2% Methanol/CH₂Cl₂). The desired white solid was obtained at 70%.

2-bromo-6-isopropylquinoline (4a). To a round bottom flask cooled to 5° C. with an ice bath, benzoyl chloride (2.33 ml, 0.02 mol) was added slowly to the vigorously stirred mixture of compound (2a) (2.5 g, 0.0133 mol), sodium hydroxide (1 g, 0.025 mol) in water (12 ml) and CHCl₂ (10 ml). After the addition is complete, the reaction mixture was left to stir for few hours. Then, the mixture was extracted from CH₂Cl₂. The combined organic layer was dried over MgSO₄. Solvent was removed under reduced pressure to obtain a white solid product. After that, the solid was mixed with POBr₃ (2.2 g, 0.007 mol) in dry toluene (20 ml) under inert atmosphere, heated to reflux overnight. After cooling to room temperature, the mixture was poured on ice, washed with saturated NaHCO₃ and extracted with CH₂Cl₂ several times. The solvent was removed under reduced pressure. The crude product was then purified using a silica gel column chromatography (50% Hexane/CH₂Cl₂). The desired white solid was obtained at 40%.

Bis(6-isopropylquinoline-2-yl)amine (5a). Compound (3a) (2.23 g, 0.012 mol) and compound (4a) (3 g, 0.012 mol) were mixed with bis(2-diphenylphosphinophenyl)ether (0.246 g, 4% mmol), t-BuONa (1.54 g, 0.016 mol), and Pd(OAc)₂ (0.1 g, 4% mmol) in a three-neck round bottom flask. The flask was subjected to three cycles of evacuation-backfilling with N₂. Dry toluene purged with N₂ was transferred to the reaction mixture using a cannula. The reaction was refluxed for 48 h at 110° C. under inert atmosphere. After that, the mixture was cooled to room temperature, extracted from CH₂Cl₂, dried over MgSO₄, and solvent removed under reduced pressure. The crude product was then purified using a silica gel column chromatography (2% Methanol/CH₂Cl₂). The desired white solid was obtained at 50%.

(α-aID). Compound (5a) (1 g, 0.0028 mol) was dissolved in dry toluene under N₂ in a three-neck round bottom flask. DIEA (1.47 ml, 0.008 mol) was slowly injected to the solution. After 30 min stirring, BF₃OEt₂ (1.39 ml, 0.0011 mol) was slowly added dropwise to the solution. The reaction was then left to reflux overnight. After cooling to room temperature, saturated solution of NaHCO₃ aq was added to the reaction mixture, followed by extraction from CH₂Cl₂. The combined organic layers were dried over MgSO₄, and solvent removed under reduced pressure. The crude product was purified by silica gel chromatography (50% Hexane/Ethyl acetate) to afford a yellow solid. The desired product was further sublimed at 190° C. under 1.2×10⁻⁶ torr.

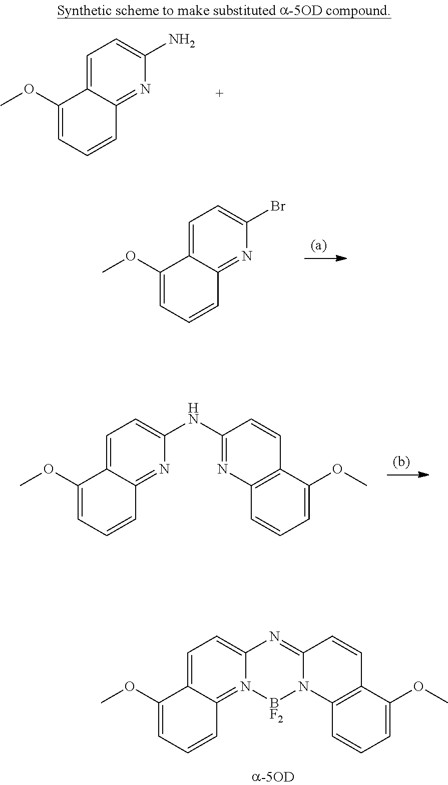

2,2′-di-5-methoxyquinolylamine (α-5OD ligand): bis(2-diphenylphosphinophenyl) ether (43 mg, 80 μmol), 2-bromo-5-methoxyquinoline (500 mg, 2.1 mmol), 2-amino-5-methoxyquinoline (365 mg, 2.1 mmol) and t-BuONa (269 mg, 2.8 mmol), and Pd(OAc)₂ (17.96 mg, 80 μmol) catalyst were added to a three-neck round bottom flask. The air free flask and refluxed in a 110° C. oil bath for 48 hours. The flask was subjected to three cycles of evacuation-backfilling with N₂. Dry toluene purged with N₂ was transferred to the reaction mixture using a cannula. The reaction was refluxed for 48 h at 110° C. under inert atmosphere. After that, the mixture was cooled to room temperature, extracted from CH₂Cl₂, dried over MgSO₄, and solvent removed under reduced pressure. The crude product was then purified using a silica gel column chromatography (2% Methanol/CH₂Cl₂).

(α-5OD). In a three-neck round bottom flask, the ligand, 2,2′-di-5-methoxyquinolylamine (α-5OD ligand) (500 mg, 1.51 mmol) was dissolved in dry toluene under N₂. DIEA (0.79 ml, 4.53 mmol) was slowly injected to the solution. After 30 min of stirring, BF₃OEt₂ (0.745 ml, 6.04 mmol) was slowly added dropwise to the solution. The reaction was then left to reflux overnight. After cooling to room temperature, saturated solution of NaHCO₃ aq was added to the reaction mixture, followed by extraction from CH₂Cl₂. The combined organic layers were dried over MgSO₄, and solvent removed under reduced pressure. The crude product was purified by silica gel chromatography (2% Methanol/CH₂Cl₂) to afford a yellow solid. The desired product was further sublimed at 200° C. under 1.2×10⁻⁶ torr.

5-methoxy-N-(quinolin-2-vl)quinolin-2-amine (αα-OD ligand): bis(2-diphenylphosphinophenyl) ether (17.23 mg, 32 μmol), 2-bromo-5-methoxyquinoline (200 mg, 0.84 mmol), 2-aminoquinoline (121 mg, 0.84 mmol) and t-BuONa (107.64 mg, 1.12 mmol), and Pd(OAc)₂ (7.18 mg, 32 μmol) catalyst were added to a three-neck round bottom flask. The air free flask and refluxed in a 110° C. oil bath for 48 hours. The flask was subjected to three cycles of evacuation-backfilling with N₂. Dry toluene purged with N₂ was transferred to the reaction mixture using a cannula. The reaction was refluxed for 48 h at 110° C. under inert atmosphere. After that, the mixture was cooled to room temperature, extracted from CH₂Cl₂, dried over MgSO₄, and solvent removed under reduced pressure. The crude product was then purified using a silica gel column chromatography (2% Methanol/CH₂Cl₂).

(αα-OD). In a three-neck round bottom flask, the ligand, 5-methoxy-N-(quinolin-2-yl)quinolin-2-amine (α-5OD ligand) (500 mg, 1.51 mmol) was dissolved in dry toluene under N₂. DIEA (0.79 ml, 4.53 mmol) was slowly injected to the solution. After 30 min of stirring, BF₃OEt₂ (0.745 ml, 6.04 mmol) was slowly added dropwise to the solution. The reaction was then left to reflux overnight. After cooling to room temperature, saturated solution of NaHCO₃ aq was added to the reaction mixture, followed by extraction from CH₂Cl₂. The combined organic layers were dried over MgSO₄, and solvent removed under reduced pressure. The crude product was purified by silica gel chromatography (2% Methanol/CH₂Cl₂) to afford a yellow solid. The desired product was further sublimed at 200° C. under 1.2×10⁻⁶ torr.

N-(isoquinolin-1-yl)-5-methoxyquinolin-2-amine (α_-OD ligand): bis(2-diphenylphosphinophenyl) ether (23.56 mg, 43.74 μmol), 2-amino-5-methoxyquinoline (200 mg, 1.15 mmol), 1-chloroisoquinoline (187.83 mg, 1.15 mmol) and t-BuONa (147.12 mg, 1.53 mmol), and Pd(OAc)₂ (9.82 mg, 43.74 μmol) catalyst were added to a three-neck round bottom flask. The air free flask and refluxed in a 110° C. oil bath for 48 hours. The flask was subjected to three cycles of evacuation-backfilling with N₂. Dry toluene purged with N₂ was transferred to the reaction mixture using a cannula. The reaction was refluxed for 48 h at 110° C. under inert atmosphere. After that, the mixture was cooled to room temperature, extracted from CH₂Cl₂, dried over MgSO₄, and solvent removed under reduced pressure. The crude product was then purified using a silica gel column chromatography (2% Methanol/CH₂Cl₂).

(α [symbol] -OD). In a three-neck round bottom flask, the ligand, N-(isoquinolin-1-yl)-5-methoxyquinolin-2-amine (α [symbol] -OD ligand) (300 mg, 1.0 mmol) was dissolved in dry toluene under N₂. DIEA (0.52 ml, 2.99 mmol) was slowly injected to the solution. After 30 min of stirring, BF₃OEt₂ (0.491 ml, 3.98 mmol) was slowly added dropwise to the solution. The reaction was then left to reflux overnight. After cooling to room temperature, saturated solution of NaHCO₃ aq was added to the reaction mixture, followed by extraction from CH₂Cl₂. The combined organic layers were dried over MgSO₄, and solvent removed under reduced pressure. The crude product was purified by silica gel chromatography (2% Methanol/CH₂Cl₂) to afford a yellow solid. The desired product was further sublimed at 200° C. under 1.2×10⁻⁶ torr.

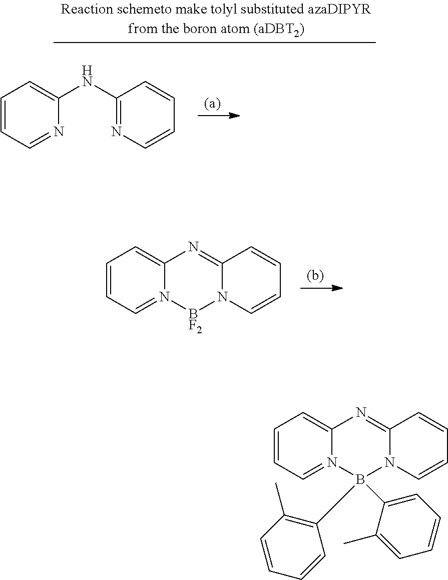

(a) azaDIPYR (aD): All reagents were purchased from Sigma Aldrich and used without purification. Anhydrous 1,2 dichloroethane was purchased from EMD Millipore. A solution of the 2,2′-dipyridylamine ligand (300 mg, 1.75mmol) in dry 1,2-dichloroethane was prepared in an N2-purged schlenk flask equipped with a magnetic stir bar and fitted with a reflux condenser. The flask was submerged in a preheated oil bath and brought to reflux, at which time 2.0 eq, boron trifluoride diethyl etherate (497.40 mg, 3.50mmol) were added dropwise. The solution was stirred for 2 hours at reflux, then cooled to room temperature and treated with 5 eq. N,N diisopropylethylamine (1.53mL, 8.70mmol). The solution was washed with water and the aqueous layer was separated and extracted three times with dichloromethane. The organic layers were combined, dried over sodium sulfate, filtered, and reduced concentrated by rotary evaporation. The products were purified by silica gel flash chromatography with the eluent 50% dichloromethane in hexanes. (b) azaDIPYRBT₂ (aDBT₂): A solution of aD (528 mg, 2.27 mmol) in dry toluene or dry THF was purged with nitrogen gas in a sealed round bottom flask equipped with a stir bar, o-Tolylmagnesium bromide (2.0M, 9.06 mL, 9.06 mmol) was added dropwise at 25° C. and allowed to stir for 12 hours. Additional o-Tolylmagnesium bromide was added and allowed to stir for 3 hours. The reaction was quenched with water and extracted three times with 100 mL of ethylacetate. The extracted organic fraction was dried using sodium sulfate, concentrated using a rotary evaporation. The product was purified by silica gel flash chromatography with the eluent 80% ethylacetate in hexanes.

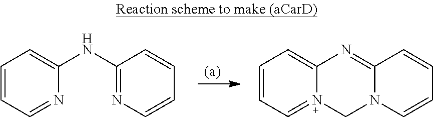

(a) azaDIPYRBPh₂ (aDBPh2): All reagents were purchased from Sigma Aldrich and used without purification. Anhydrous 1,2 dichloroethane was purchased from EMD Millipore. A solution of the 2,2′-dipyridylamine ligand (800 mg, 4.67 mmol) and diphenylboronic anhydride (1.62 g, 4.67 mmol) in dry 1,2-dichloroethane was prepared in an N2-purged schlenk flask equipped with a magnetic stir bar and fitted with a reflux condenser. The flask was submerged in a preheated oil bath and brought to reflux for 16 hours. The solution was washed with water and the aqueous layer was separated and extracted three times with dichloromethane. The organic layers were combined, dried over sodium sulfate, filtered, and reduced concentrated by rotary evaporation. The product was purified by silica gel flash chromatography with 100% ethylacetate then 100% acetone.

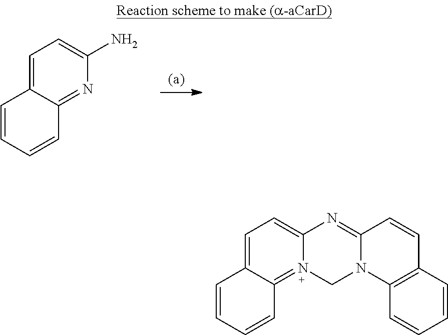

(a) azaCARDIPYR (α-CarD): A reported procedure was followed (cite); 2,2′-dipyridylamine (0.94 mg, 9.99mmol) and diiodomethane(4.01 g, 14.98 mmol) was refluxed in dry acetonitrile for 28 hours under nitrogen. The reaction mixture was cooled to room temperature and allowed to sit in air for 2 hours. A solid precipitated out of solution and was isolated as the pure product after filtering and rinsing with methanol.

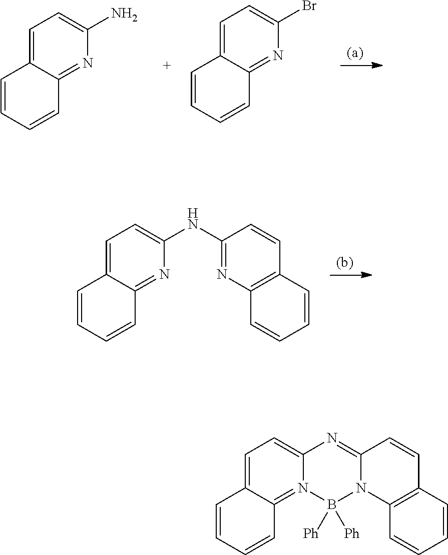

(a) α-azaCARDIPYR (α-aCarD): A reported procedure was followed (cite); 2,2′-diquinolylamine (1.00 g, 6.94) and diiodomethane (2.79, 10.40 mmol) was refluxed in dry acetonitrile for 28 hours under nitrogen. The reaction mixture was cooled to room temperature and allowed to sit in air for 2 hours. A solid precipitated out of solution and was isolated as the pure product after filtering and rinsing with methanol.

(a) 2,2′-diquinolylamine (α-aD ligand): A reported procedure was followed (cite); bis(2-diphenylphosphinophenyl) ether(395.03 mg, 733.48 μmol), 2-bromoquinoline(3.00 g, 18.34 mmol), 2-aminoquinoline (2.78 g, 19.25 mmol) and t-BuONa (2.47 g, 25.67 mmol) were purged with nitrogen gas in a re-sealable shlenk flask where a degassed dry toluene is cannula transferred. Pd(OAc)₂ (164.67, 733.48 μmol) catalyst was added to the air free flask and refluxed in a 110° C. oil bath for 24 hours. The reaction mixture is cooled to room temperature and diluted with THF and ethyl ether. The solid precipitate was filtered, concentrated, and purified via silica gel column chromatography (2% MeOH/CH₂Cl₂). A white solid is isolated upon purification (40-80% yield).

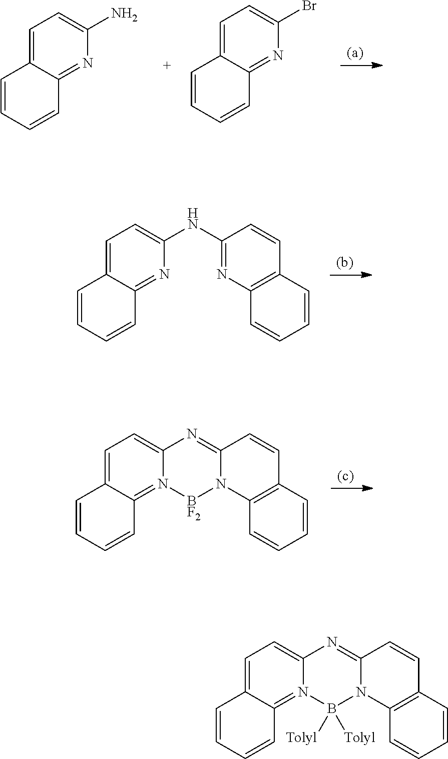

(b) α-azaDIPYRBT₂ (α-aDBT₂): All reagents were purchased from Sigma Aldrich and used without purification. Anhydrous 1,2 dichloroethane was purchased from EMD Millipore. A 15 mM solution of the 2,2′-diquinolylamine ligand and diphenylboronic anhydride in dry 1,2-dichloroethane was prepared in an N2-purged schlenk flask equipped with a magnetic stir bar and fitted with a reflux condenser. The flask was submerged in a preheated oil bath and brought to reflux for 16 hours. The solution was washed with water and the aqueous layer was separated and extracted three times with dichloromethane. The organic layers were combined, dried over sodium sulfate, filtered, and reduced concentrated by rotary evaporation. The product was purified by silica gel flash chromatography with 100% ethylacetate then 100% acetone.

a) 2,2′-diquinolylamine (α-aD ligand): A reported procedure was followed (cite); bis(2-diphenylphosphinophenyl) ether(395.03 mg, 733.48 μmol), 2-bromoquinoline(3.00 g, 18.34 mmol), 2-aminoquinoline (2.78 g, 19.25 mmol) and t-BuONa (2.47 g, 25.67 mmol) were purged with nitrogen gas in a re-sealable shlenk flask where a degassed dry toluene is cannula transferred. Pd(OAc)₂ (164.67, 733.48 μmol) catalyst was added to the air free flask and refluxed in a 110° C. oil bath for 24 hours. The reaction mixture is cooled to room temperature and diluted with THF and ethyl ether. The solid precipitate was filtered, concentrated, and purified via silica gel column chromatography (2% MeOH/CH₂Cl₂). A white solid is isolated upon purification (40-80% yield).

(b) α-azaDIPYR (α-aD): All reagents were purchased from Sigma Aldrich and used without purification. Anhydrous 1,2 dichloroethane was purchased from EMD Millipore. A 15 mM solution of 2,2′-diquinolylamine in dry 1,2-dichloroethane was prepared in an N2-purged schlenk flask equipped with a magnetic stir bar and fitted with a reflux condenser. The flask was submerged in a preheated oil bath and brought to reflux, at which time 2.0 eq, boron trifluoride diethyl etherate were added dropwise. The solution was stirred for 2 hours at reflux, then cooled to room temperature and treated with 5 eq. N,N diisopropylethylamine, causing the precipitate to dissolve. The solution was washed with water and the aqueous layer was separated and extracted three times with dichloromethane. The organic layers were combined, dried over sodium sulfate, filtered, and reduced concentrated by rotary evaporation. The products were purified by silica gel flash chromatography with the eluent 2% MeOH/CH₂Cl₂ solvent mixture in hexanes.

(c) α-azaDIPYRBT₂ (α-aDBT₂): A solution of α-aD (528 mg, 1.59 mmol) in dry toluene or dry THF was purged with nitrogen gas in a sealed round bottom flask equipped with a stir bar, o-Tolylmagnesium bromide (2.0M, 3.18 mL, 6.36 mmol) was added dropwise at 25° C. and allowed to stir for 12 hours. Additional o-Tolylmagnesium bromide was added and allowed to stir for 3 hours. The reaction was quenched with water and extracted three times with 100 mL of ethylacetate. The extracted organic fraction was dried using sodium sulfate, concentrated using a rotary evaporation. The product was purified by silica gel flash chromatography with the eluent 70% ethylacetate in hexanes.

It is understood that the various embodiments described herein are by way of example only, and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting.

1. An organic light emitting diode (OLED) comprising:
an anode;
a cathode; and
a hybrid first emissive layer disposed between the anode and the cathode, wherein the hybrid emissive layer comprises:
a first material having a triplet state energy level T1_{H} and a singlet state energy level S1_{H}; a second material having a triplet state energy level T1_{F} and a singlet state energy level S1_{F}; and a third material having a triplet state energy level T1_{P} and a single state energy level S1_{P};
wherein:
$$T {1}_{F} \geq T {1}_{h} ; S {1}_{F} \leq S {1}_{H} ; and T {1}_{P} < T {1}_{H} .$$

2-21. (canceled)
